(19) United States
(12) Patent Application Publication
(10) Pub. No.: US 2013/0184257 A1
(43) Pub. Date: Jul. 18, 2013

(54) PIPERIDINYL-SUBSTITUTED LACTAMS AS GPR119 MODULATORS

(71) Applicants: Thomas Daniel Aicher, Superior, CO (US); Josef R. Bencsik, LaSalle (CA); Steven Armen Boyd, Longmont, CO (US); Kevin Ronald Condroski, Wallingford, CT (US); Jay Bradford Fell, Boulder, CO (US); John P. Fischer, Boulder, CO (US); Ronald Jay Hinklin, Boulder, CO (US); Scott Alan Pratt, Longmont, CO (US)

(72) Inventors: Thomas Daniel Aicher, Superior, CO (US); Josef R. Bencsik, LaSalle (CA); Steven Armen Boyd, Longmont, CO (US); Kevin Ronald Condroski, Wallingford, CT (US); Jay Bradford Fell, Boulder, CO (US); John P. Fischer, Boulder, CO (US); Ronald Jay Hinklin, Boulder, CO (US); Scott Alan Pratt, Longmont, CO (US)

(21) Appl. No.: 13/823,041

(22) PCT Filed: Sep. 15, 2011
(86) PCT No.: PCT/US11/51821

§ 371 Date: Mar. 13, 2013

Related U.S. Application Data
(60) Provisional application No. 61/383,799, filed on Sep. 17, 2010.

(51) Int. Cl. C07D 413/14; C07D 401/04; C07D 401/14; C07D 211/76

(52) U.S. Cl. CPC C07D 413/14; C07D 211/76; C07D 401/04; C07D 401/14
USPC 514/212.08; 546/209; 514/326; 546/187; 514/316; 546/188; 546/208; 546/194; 514/318; 540/524

(57) ABSTRACT

Compounds of Formula (I) and pharmaceutically acceptable salts thereof in which X¹, X², L, R³, R⁴, R⁵, R⁷ and n have the meanings given in the specification, are modulators of GPR119 and are useful in the treatment or prevention of diseases such as such as, but not limited to, type 2 diabetes, diabetic complications, symptoms of diabetes, metabolic syndrome, obesity, dyslipidemia, and related conditions.

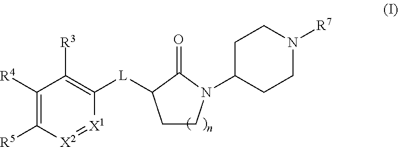

The present invention relates to novel compounds, to pharmaceutical compositions comprising the compounds, to processes for making the compounds, and to the use of the compounds in therapy. More particularly, it relates to certain piperidinyl-substituted lactams which are modulators of GPR119 and are useful in the treatment or prevention of diseases such as, but not limited to, type 2 diabetes, diabetic complications, symptoms of diabetes, metabolic syndrome, obesity, dyslipidemia, and related conditions. In addition, the compounds are useful in decreasing food intake, decreasing weight gain, and increasing satiety in mammals.

Diabetes is diagnosed by elevated fasting plasma glucose levels ≧126 mg/dL or by plasma glucose levels after an oral glucose tolerance test ≧200 mg/dL. Diabetes is associated with the classic symptoms of polydipsia, polyphagia and polyuria (The Expert Committee on the Diagnosis and Classification of Diabetes Mellitus, Diabetes Care, 1998, 21, S5-19). Of the two major forms of diabetes, insulin dependent diabetes mellitus (Type I) accounts for 5-10% of the diabetic population. Type I diabetes is characterized by near total beta cell loss in the pancreas and little or no circulating insulin. Non-insulin dependent diabetes mellitus (Type 2 diabetes) is the more common form of diabetes. Type 2 diabetes is a chronic metabolic disease that develops from a combination of insulin resistance in the muscle, fat, and liver and from partial beta cell loss in the pancreas. The disease progresses with the inability of the pancreas to secrete sufficient insulin to overcome such resistance. Uncontrolled type 2 diabetes is associated with an increased risk of heart disease, stroke, neuropathy, retinopathy and nephropathy among other diseases.

Obesity is a medical condition characterized by high levels of adipose tissue in the body. Body mass index is calculated by dividing weight by height squared (BMI=kg/m²), where a person with a BMI of ≧30 is considered obese and medical intervention is recommended (For the Clinical Efficacy Assessment Subcommittee of the American College of Physicians. Pharmacological and surgical management of obesity in primary care: a clinical practice guideline from the American College of Physicians. Ann Intern Med, 2005, 142, 525-531). The main causes of obesity are increased calorie intake accompanied with a lack of physical activity and genetic predisposition. Obesity leads to an increased risk of many diseases including, but not limited to, diabetes, heart disease, stroke, dementia, cancer, and osteoarthritis.

Metabolic syndrome is present when a group of risk factors are found in a mammal (Grundy, S. M.; Brewer, H. B. Jr; et al., Circulation, 2004, 109, 433-438). Abdominal obesity, dyslipidemia, high blood pressure and insulin resistance predominate in this disease. Similar to obesity, metabolic syndrome results from increased calorie intake, physical inactivity, and aging. Of major concern is that this condition can lead to coronary artery disease and type 2 diabetes.

Clinically there are a number of treatments currently being used to lower blood glucose in type 2 diabetic patients. Metformin (De Fronzo, R. A.; Goodman, A. M., N. Engl. J. Med., 1995, 333, 541-549) and the PPAR agonists (Wilson, T. M., et al., J. Med. Chem., 1996, 39, 665-668) partially ameliorate insulin resistance by improving glucose utilization in cells. Treatment with sulfonylureas (Blickle, J. F., Diabetes Metab. 2006 32, 113-120) has been shown to promote insulin secretion by affecting the pancreatic KATP channel; however, the increase in insulin is not glucose dependent and such treatment can lead to hypoglycemia. The recently approved DPP4 inhibitors and GLP-1 mimetics promote insulin secretion by the beta cell through an incretin mechanism, and administration of these agents causes insulin release in a glucose dependent manner (Vahl, T. P., D'Alessio, D. A., Expert Opinion on Invest. Drugs, 2004, 13, 177-188). However, even with these newer treatments, it is difficult to achieve precise control of blood glucose levels in type 2 diabetic patients in accordance with the guidelines recommended by the American Diabetes Association.

GPR119 is a Gs-coupled receptor that is predominately expressed in the pancreatic beta cells and in the enteroendocrine K and L cells of the GI tract. In the gut, this receptor is activated by endogenous lipid-derived ligands such as oleoylethanolamide (Lauffer, L. M., et al., Diabetes, 2009, 58, 1058-1066). Upon activation of GPR119 by an agonist, the enteroendocrine cells release the gut hormones glucagon like peptide 1 (GLP-1), glucose-dependent insulinotropic peptide (GIP), and peptide YY (PYY) among others. GLP-1 and GIP have multiple mechanisms of action that are important for controlling blood glucose levels (Parker, H. E., et al., Diabetologia, 2009, 52, 289-298). One action of these hormones is to bind to GPCRs on the surface of beta cells leading to a rise in intracellular c-AMP levels. This rise results in a glucose dependent release of insulin by the pancreas (Drucker, D. J. J. Clin. Investigation, 2007, 117, 24-32; Winzell, M. S., Pharmacol. and Therap. 2007, 116, 437-448). In addition, GLP-1 and GIP have been shown to increase beta cell proliferation and decrease the rate of apoptosis in vivo in animal models of diabetes and in vitro with human beta cells (Farilla, L.; et al., Endocrinology, 2002, 143, 4397-4408; Farilla, L.; et al., Endocrinology, 2003, 144 5149-5158; and Hughes, T. E., Current Opin. Chem. Biol., 2009, 13, 1-6). Current GLP-1 mechanism based therapies, such as sitagliptin and exenatide, are clinically validated to improve glucose control in type 2 diabetic patients.

GPR119 receptors are also expressed directly on the pancreatic beta cells. A GPR119 agonist can bind to the pancreatic GPR119 receptor and cause a rise in cellular c-AMP levels consistent with a Gs-coupled GPCR signaling mechanism. The increased c-AMP then leads to a release of insulin in a glucose dependent manner. The ability of GPR119 agonists to enhance glucose-dependent insulin release by direct action on the pancreas has been demonstrated in vitro and in vivo (Chu Z., et al., Endocrinology 2007, 148:2601-2609). This dual mechanism of action of the release of incretin hormones in the gut and binding directly to receptors on the pancreas may offer an advantage for GPR119 agonists over current therapies for treating diabetes.

GPR119 agonists, by increasing the release of PYY, may also be of benefit in treating many of comorbidities associated with diabetes and to treat these diseases in the absence of diabetes. Administration of PYY₃₋₃₆ has been reported to reduce food intake in animals (Batterham, R. L., et al., Nature, 2002, 418, 650-654), increase satiety and decrease food intake in humans (Batterham, R. L., et al., Nature, 2002, 418, 650-654), increase resting body metabolism (Sloth B., et al., Am. J. Physiol. Endocrinol. Metab., 2007, 292, E1062-1068 and Guo, Y., et al., Obesity, 2006, 14, 1562-1570), increase fat oxidation (Adams, S. H., et al., J. Nutr., 2006, 136, 195-201 and van den Hoek, A. M., et al., Diabetes, 2004, 53, 1949-1952), increase thyroid hormone activity, and increase adiponectin levels. PYY release caused by GPR119 agonists can therefore be beneficial in treating the metabolic syndrome and obesity.

Several classes of small molecule GPR119 agonists are known (Fyfe, M. T. E. et al., Expert Opin. Drug. Discov., 2008, 3(4), 403-413; Jones, R. M., et al., Expert Opin. Ther. Patents, 2009, 19(10), 1339-1359).

There remains, however, a need for compounds and methods for the treatment or prevention of diabetes, dyslipidemia, diabetic complications, and obesity.

### SUMMARY OF THE INVENTION

It has now been found that certain novel piperidinyl-substituted lactams are modulators of GPR119 and are useful for treating type 2 diabetes, diabetic complications, metabolic syndrome, obesity, dyslipidemia, and related conditions.

Accordingly, in one aspect of the present invention there is provided compounds having the general Formula I

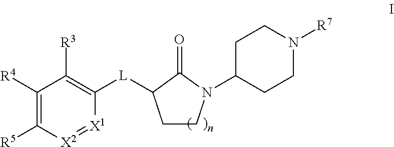

and pharmaceutically acceptable salts thereof, wherein X¹, X², L, R³, R⁴, R⁵, R⁷ and n are as defined herein.

In another aspect of the invention, there are provided pharmaceutical compositions comprising a compound of Formula I and a pharmaceutically acceptable carrier, diluent or excipient.

In another aspect of the invention, there is provided a method of treating a disease or condition selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis in a mammal, which comprises administering to said mammal a therapeutically effective amount of a compound of Formula I or a pharmaceutically acceptable salt thereof. In one embodiment, the method comprises administering a compound of Formula I in combination with one or more additional drugs. In one embodiment, the additional drug is a biguanide. In one embodiment, the additional drug is a DPP4 inhibitor.

In another aspect of the invention, there is provided the use of a compound of Formula I in the treatment of a disease or condition selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis.

In another aspect of the invention, there is provided compounds of Formula I or pharmaceutically acceptable salts thereof, for use in therapy.

In another aspect of the invention, there is provided compounds of Formula I or pharmaceutically acceptable salts thereof, for use in treating a disease or condition selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis, diabetic complications, metabolic syndrome (including hyperglycemia, impaired glucose tolerance, and insulin resistance), obesity, dyslipidemia, dyslipoproteinemia.

In another aspect of the invention, there is provided compounds of Formula I or pharmaceutically acceptable salts thereof, for use in treating a disease or condition selected from type 2 diabetes, symptoms of diabetes,

Another aspect of the invention provides intermediates for preparing compounds of Formula I. In one embodiment, certain compounds of Formula I may be used as intermediates for the preparation of other compounds of Formula I.

Another aspect of the invention includes processes for preparing, methods of separation, and methods of purification of the compounds described herein.

### DETAILED DESCRIPTION OF THE INVENTION

One embodiment of this invention provides compounds of the general Formula I

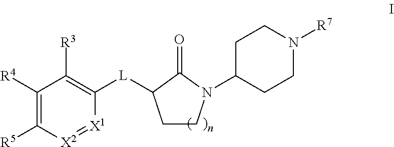

and pharmaceutically acceptable salts thereof, wherein:

L is O or NR^{x};

R^{x} is H or (1-3C)alkyl;

X¹ is N or CR¹ and X² is N or CR², wherein only one of X¹ and X² may be N;

R¹, R², R³ and R⁴ are independently selected from H, halogen, CF₃, (1-6C)alkyl and (1-6C)alkoxy;

R⁵ is (1-3C alkyl)sulfonyl, (3-6C cycloalkyl)sulfonyl, (cyclopropylmethyl)sulfonyl, phenylsulfonyl, CN, Br, CF₃, or tetrazolyl optionally substituted with (1-3C)alkyl;

R⁷ is selected from

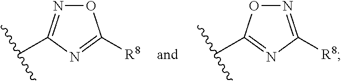

R⁸ is (1-6C)alkyl, fluoro(1-6C)alkyl, difluoro(1-6C)alkyl, trifluoro(1-6C)alkyl, trichloro(1-6C)alkyl, Cyc¹, Ar¹, hetCyc¹ or hetAr¹;

Cyc¹ is (3-6C)cycloalkyl optionally substituted with CF₃;

Ar¹ is phenyl optionally substituted with one or more groups independently selected from halogen, CF₃, (1-4C)alkyl and (1-4C)alkoxy;

hetCyc¹ is a 5-6 membered heterocycle having a ring nitrogen atom and optionally substituted with one or more groups independently selected from halogen, CF₃, (1-4C)alkyl and (1-4C)alkoxy;

hetAr¹ is a 6-membered heteroaryl having a ring nitrogen atom and optionally substituted with one or more groups independently selected from halogen, CF₃, (1-4C)alkyl and (1-4C)alkoxy; and

n is 1, 2 or 3.

In one embodiment of Formula I, n is 1.

In one embodiment of Formula I, n is 2.

In one embodiment of Formula I, n is 3.

In one embodiment of Formula I, L is O.

In one embodiment of Formula I, L is NR^{x}.

In one embodiment, L is NH.

In one embodiment, L is N(1-3C)alkyl. Particular examples include NCH₃ and NCH₂CH₃.

In one embodiment, R¹ is H, F, Cl or CF₃.

In one embodiment, R¹ is H, F or Cl.

In one embodiment, R¹ is H.

In one embodiment, R¹ is F.

In one embodiment, R¹ is Cl.

In one embodiment, R¹ is CF₃.

In one embodiment, R² is H, F or Me.

In one embodiment, R² is H.

In one embodiment, R² is F.

In one embodiment, R² is Me.

In one embodiment, R³ is H, F, Cl or CF₃.

In one embodiment, R³ is H.

In one embodiment, R³ is F.

In one embodiment, R³ is Cl.

In one embodiment, R³ is CF₃.

In one embodiment, R⁴ is H, Me, F, or Cl.

In one embodiment, R⁴ is H.

In one embodiment, R⁴ is Me.

In one embodiment, R⁴ is F.

In one embodiment, R⁴ is Cl.

In one embodiment, R¹ and R² are independently selected from H, F and Cl; and R³ and R⁴ are independently selected from H, Me, F, Cl and CF₃.

In one embodiment, R¹ and R³ are F; and R² and R⁴ are H.

In one embodiment, R¹ and R⁴ are H; and R² and R³ are F.

In one embodiment, R¹, R² and R⁴ are H; and R³ is F.

In one embodiment of Formula I, the residue:

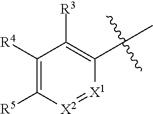

of Formula I, wherein the wavy line represents the point of attachment of the residue in Formula I, is selected from a residue wherein X¹ is CR¹ and X² is CR², such that the residue can be represented as:

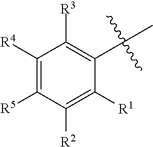

wherein R¹, R², R³, R⁴ and R⁵ are as defined for Formula I.

In one embodiment, R¹, R², R³ and R⁴ are independently selected from H, F, Cl, CF₃, methyl, ethyl, propyl, isopropyl, methoxy, ethoxy, propoxy and isopropoxy.

In one embodiment of Formula I, R¹, R², R³ and R⁴ are independently selected from H, (1-6C)alkyl, CF₃ and halogen.

In one embodiment, R¹ and R² are independently selected from H, F and Cl, and R³ and R⁴ are independently selected from H, Me, F, Cl and CF₃.

In one embodiment, R¹ is H or F, R² is H, F or Cl, R³ is H, F, or CF₃, and R⁴ is H, Me, F, or Cl.

In one embodiment, R¹, R², R³ and R⁴ are independently selected from H, Me and halogen.

In one embodiment, R¹, R², R³ and R⁴ are independently selected from H and halogen.

In one embodiment, R¹, R², R³ and R⁴ are independently selected from H and F.

In one embodiment, R¹, R² and R⁴ are H, and R³ is F.

In one embodiment, R¹ and R³ are F, and R² and R⁴ are H.

In one embodiment, R¹ and R⁴ are H, and R² and R³ are F.

In one embodiment, R¹, R² and R³ are H, and R⁴ is F.

In one embodiment, R¹ and R⁴ are H, R² is Cl and R³ is F.

In one embodiment, R¹ and R⁴ are H, R² is Me and R³ is F.

In one embodiment, R¹, R² and R⁴ are H, and R³ is CF₃.

In one embodiment, R¹, R² and R³ are H, and R⁴ is Cl.

In one embodiment, R¹ is F, R² and R³ are H, and R⁴ is Me.

In one embodiment of Formula I, the residue:

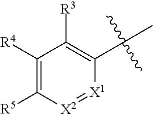

of Formula I, wherein the wavy line represents the point of attachment of the residue to “L” in Formula I, is selected from a residue wherein X¹ is N and X² is CR², such that the residue can be represented as:

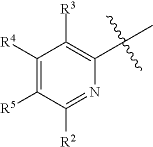

wherein R², R³, R⁴ and R⁵ are as defined for Formula I. In one embodiment, R², R³ and R⁴ are independently selected from H, F, Cl, CF₃, methyl, ethyl, propyl, isopropyl, methoxy, ethoxy, propoxy and isopropoxy. In one embodiment, R², R³ and R⁴ are independently selected from H, halogen, CF₃, and (1-6C)alkyl. In one embodiment, R², R³ and R⁴ are independently selected from H, halogen, and (1-6C)alkyl. In one embodiment, R², R³ and R⁴ are independently selected from H, F, Cl and Me. In one embodiment, R², R³ and R⁴ are independently selected from H or Cl. In one embodiment, R² is H. In one embodiment, R³ is H. In one embodiment, R³ is Cl. In one embodiment, R⁴ is H. In one embodiment, R², R³ and R⁴ are each H. In one embodiment, R² and R⁴ are H and R³ is Cl.

In one embodiment of Formula I, the residue:

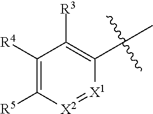

of Formula I, wherein the wavy line represents the point of attachment of the residue to “L” in Formula I, is selected from a residue wherein X¹ is CR¹ and X² is N, such that the residue can be represented as:

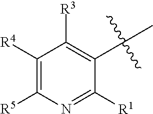

wherein R¹, R³, R⁴ and R⁵ are as defined for Formula I. In one embodiment, R¹, R³ and R⁴ are independently selected from H, F, Cl, CF₃, methyl, ethyl, propyl, isopropyl, methoxy, ethoxy, propoxy and isopropoxy. In one embodiment, R¹, R³ and R⁴ are independently selected from H, halogen, CF₃, and (1-6C)alkyl. In one embodiment, R¹, R³ and R⁴ are independently selected from H, halogen, and (1-6C)alkyl. In one embodiment, R¹, R³ and R⁴ are independently selected from H, F, Cl and Me. In one embodiment, R¹, R³ and R⁴ are independently selected from H or Cl. In one embodiment, R¹ is H. In one embodiment, R³ is H. In one embodiment, R³ is Cl. In one embodiment, R⁴ is H. In one embodiment, each of R¹, R³ and R⁴ is H.

In one embodiment of Formula I, X¹ is N or CR¹ and X² is N or CR², wherein only one of X¹ and X² may be N; R¹, R², R³ and R⁴ are independently selected from H, halogen, CF₃, (1-6C)alkyl and (1-6C)alkoxy; and R⁵ is (1-3C alkyl)sulfonyl, (3-6C cycloalkyl)sulfonyl, (cyclopropylmethyl)sulfonyl-, phenylsulfonyl-, CN, Br, CF₃, or tetrazolyl optionally substituted with (1-3C)alkyl.

In one embodiment of Formula I, R⁵ is selected from (1-3C alkyl)sulfonyl, (3-6C cycloalkyl)sulfonyl, (cyclopropylmethyl)sulfonyl, and phenylsulfonyl (C₆H₅SO₂—).

In one embodiment, R⁵ is (1-3C alkyl)sulfonyl. Examples include CH₃SO₂— and CH₃CH₂SO₂—, CH₃CH₂CH₂SO₂— and (CH₃)₂CHSO₂—. Particular examples include CH₃SO₂— and CH₃CH₂SO₂—. In one embodiment, R⁵ is CH₃SO₂—. In one embodiment, R⁵ is CH₃CH₂SO₂—.

In one embodiment, R⁵ is (3-6C cycloalkyl)sulfonyl. An example is (cyclopropyl)SO₂—.

In one embodiment, R⁵ is (cyclopropylmethyl)sulfonyl which can be represented by the structure:

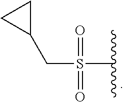 [SMILES: *S(=O)(=O)CC1CC1]

In one embodiment, R⁵ is phenylsulfonyl (C₆H₅SO₂—).

In one embodiment, R⁵ is selected from CN, Br and CF₃.

In one embodiment, R⁵ is CN.

In one embodiment, R⁵ is Br.

In one embodiment, R⁵ is CF₃.

In one embodiment, R⁵ is tetrazolyl optionally substituted with (1-3C)alkyl.

In one embodiment, R⁵ is tetrazolyl optionally substituted with methyl. Particular examples of R⁵ include groups having the structures:

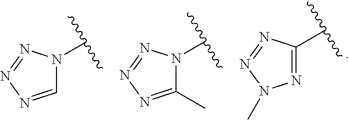

Particular examples of the group having the structure

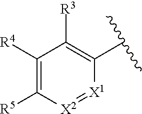

include the following structures:

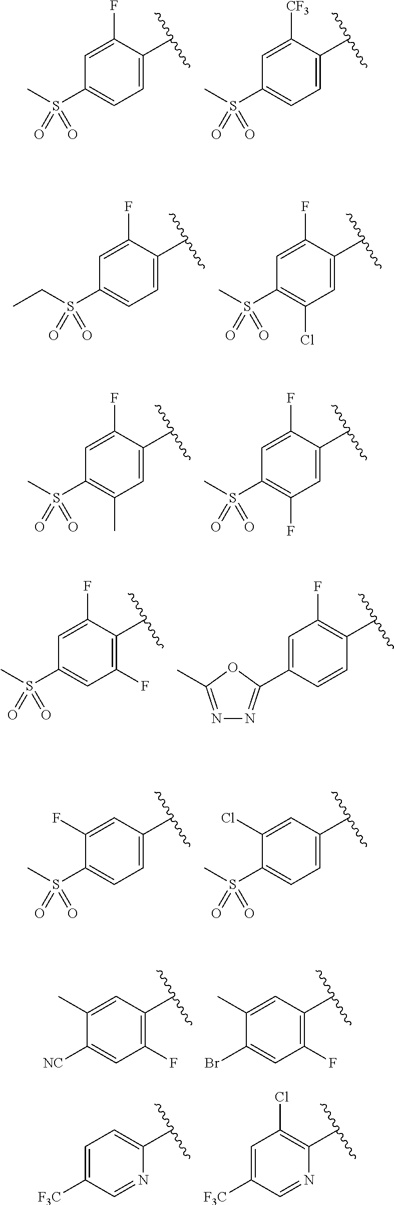

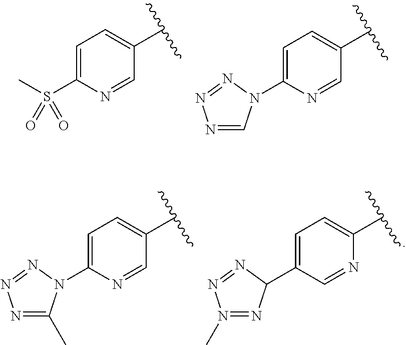

In one embodiment, the group having the structure

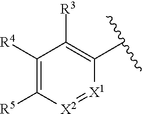

is selected from the structures:

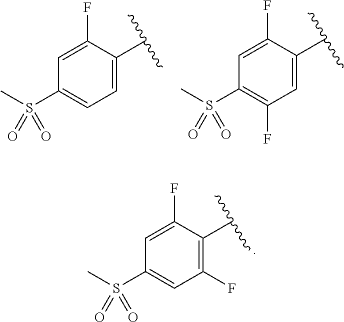 [SMILES: *c1ccc(S(C)(=O)=O)cc1F ; *c1cc(F)c(S(C)(=O)=O)cc1F ; *c1c(F)cc(S(C)(=O)=O)cc1F]

In one embodiment, R⁷ has the structure:

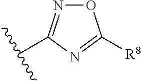

wherein R⁸ is as defined for Formula I.

In one embodiment, R⁷ has the structure:

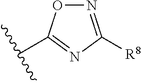

wherein R⁸ is as defined for Formula I.

In one embodiment of Formula I, R⁸ is (1-6C)alkyl. In one embodiment, R⁸ is methyl, ethyl, propyl, sec-propyl, butyl, isobutyl or tert-butyl. In one embodiment, R⁸ is ethyl, isopropyl, sec-butyl or tert-butyl. In one embodiment, R⁸ is isopropyl.

In one embodiment of Formula I, R⁸ is fluoro(1-6C)alkyl. In one embodiment, R⁸ is 2-fluoropropyl.

In one embodiment of Formula I, R⁸ is difluoro(1-6C)alkyl. In one embodiment, R⁸ is difluoromethyl, 1,1-difluoroethyl or 1,1-difluoropropyl.

In one embodiment of Formula I, R⁸ is trifluoro(1-6C)alkyl. In one embodiment, R⁸ is trifluoromethyl or 1,1-dimethyl-2,2-difluoroethyl.

In one embodiment of Formula I, R⁸ is trichloro(1-6C)alkyl. In one embodiment, R⁸ is trichloromethyl.

In one embodiment of Formula I, R⁸ is Cyc¹. In one embodiment, R⁸ is cyclopropyl, cyclobutyl or cyclopentyl optionally substituted with CF₃. In one embodiment, R⁸ is cyclopropyl, 1-(trifluoromethyl)cyclopropyl, cyclobutyl or cyclopentyl.

In one embodiment of Formula I, R⁸ is Ar¹. In one embodiment, Ar¹ is phenyl optionally substituted with one or more groups independently selected from F, Cl, CF₃, methyl, ethyl and methoxy. In one embodiment, R⁸ is phenyl.

In one embodiment of Formula I, R⁸ is hetCyc¹. In one embodiment, hetCyc¹ is an N-linked heterocycle, that is, hetCyc¹ is coupled to the R⁷ group of Formula I through a ring nitrogen atom of the hetCyc¹ group. In one embodiment, R⁸ is pyrrolidinyl optionally substituted with one or more groups independently selected from F, Cl, CF₃, methyl, ethyl and methoxy. In one embodiment R⁸ is pyrrolidin-1-yl.

In one embodiment of Formula I, R⁸ is hetAr¹. In one embodiment, R⁸ is pyridyl optionally substituted with one or more groups independently selected from F, Cl, CF₃, methyl, ethyl and methoxy. In one embodiment, R⁸ is pyrid-2-yl.

In one embodiment, R⁷ has the structure:

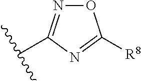

wherein R⁸ is selected from (1-6C)alkyl, fluoro(1-6C)alkyl, difluoro(1-6C)alkyl, trifluoro(1-6C)alkyl and trichloro(1-6C)alkyl. In one embodiment, R⁸ is selected from ethyl, isopropyl, propyl, sec-propyl, tert-butyl, 2-fluoropropyl, difluoromethyl, 1,1-difluoroethyl, 1,1-difluoropropyl, trifluoromethyl and 1,1-dimethyl-2,2-difluoroethyl. In one embodiment, R⁸ is selected from ethyl, isopropyl, propyl, sec-propyl and tert-butyl. In one embodiment, R⁸ is selected from 2-fluoropropyl, difluoromethyl, 1,1-difluoroethyl, 1,1-difluoropropyl, trifluoromethyl and 1,1-dimethyl-2,2-difluoroethyl.

In one embodiment, R⁷ has the structure:

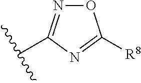

wherein R⁸ is selected from Cyc¹, Ar¹, hetCyc¹ and hetAr¹. In one embodiment, R⁸ is selected from cyclopropyl, 1-(trifluoromethyl)cyclopropyl, cyclobutyl, cyclopentyl, phenyl, pyrrolidin-1-yl and pyrid-2-yl.

In one embodiment, R⁷ has the structure:

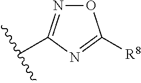

wherein R⁸ is selected from selected from ethyl, isopropyl, propyl, sec-propyl, tert-butyl, 2-fluoropropyl, difluoromethyl, 1,1-difluoroethyl, 1,1-difluoropropyl, trifluoromethyl, 1,1-dimethyl-2,2-difluoroethyl, cyclopropyl, 1-(trifluoromethyl)cyclopropyl, cyclobutyl, cyclopentyl, phenyl, pyrrolidin-1-yl or pyrid-2-yl.

In one embodiment, R⁷ is selected from the structures:

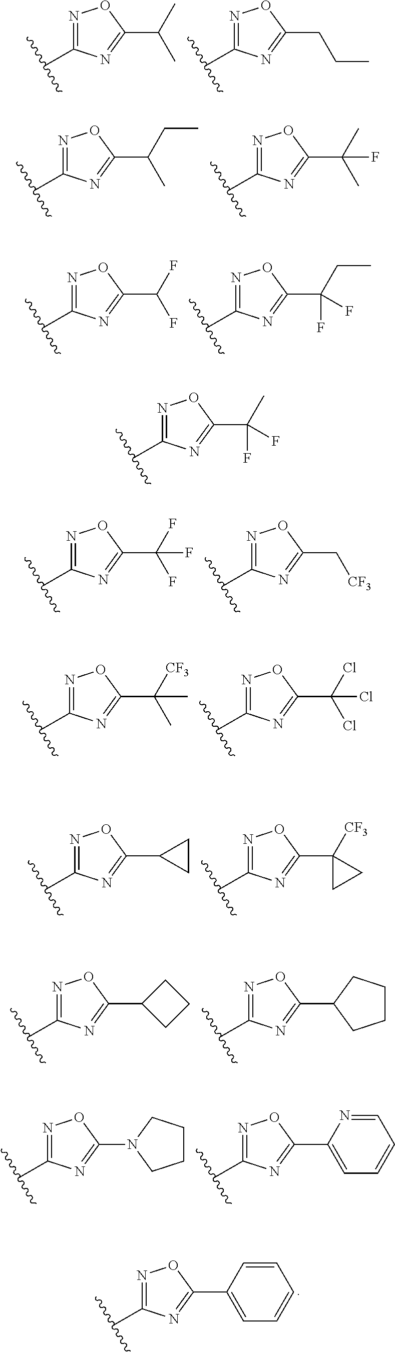

In one embodiment, R⁷ has the structure:

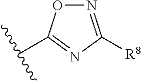

wherein R⁸ is selected from (1-6C)alkyl, fluoro(1-6C)alkyl, difluoro(1-6C)alkyl, trifluoro(1-6C)alkyl and trichloro(1-6C)alkyl. In one embodiment, R⁸ is selected from ethyl, isopropyl, propyl, sec-propyl, tert-butyl, 2-fluoropropyl, difluoromethyl, 1,1-difluoroethyl, 1,1-difluoropropyl, trifluoromethyl and 1,1-dimethyl-2,2-difluoroethyl. In one embodiment, R⁸ is selected from ethyl, isopropyl, propyl, sec-propyl and tert-butyl. In one embodiment, R⁸ is selected from 2-fluoropropyl, difluoromethyl, 1,1-difluoroethyl, 1,1-difluoropropyl, trifluoromethyl and 1,1-dimethyl-2,2-difluoroethyl.

In one embodiment, R⁷ has the structure:

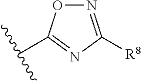

wherein R⁸ is selected from Cyc¹, Ar¹, hetCyc¹ and hetAr¹. In one embodiment, R⁸ is selected from cyclopropyl, 1-(trifluoromethyl)cyclopropyl, cyclobutyl, cyclopentyl, phenyl, pyrrolidin-1-yl and pyrid-2-yl.

In one embodiment, R⁷ has the structure:

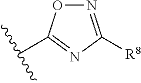

wherein R⁸ is selected from ethyl, isopropyl, propyl, sec-propyl, tert-butyl, 2-fluoropropyl, difluoromethyl, 1,1-difluoroethyl, 1,1-difluoropropyl, trifluoromethyl, 1,1-dimethyl-2,2-difluoroethyl, cyclopropyl, 1-(trifluoromethyl)cyclopropyl, cyclobutyl, cyclopentyl, phenyl, pyrrolidin-1-yl or pyrid-2-yl.

In one embodiment, R⁷ is selected from the structures:

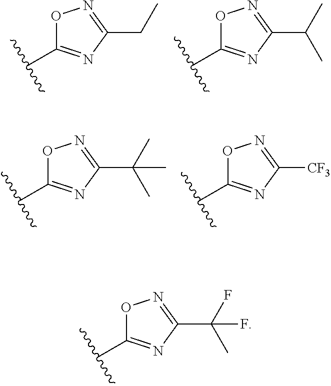

In one embodiment, R⁷ is selected from the structures:

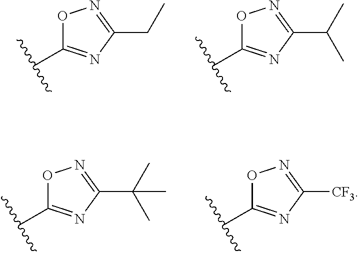

In embodiment, compounds of Formula I include compounds of Formula IA and pharmaceutically acceptable salts thereof, wherein:

L is O or NR^{x};

R^{x} is H or (1-3C)alkyl;

X¹ is CR¹ and X² is CR²;

R¹, R², R³ and R⁴ are independently selected from H and halogen;

R⁵ is (1-3C alkyl)sulfonyl;

R⁷ is selected from

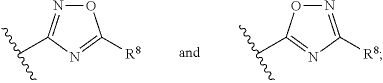

R⁸ is (1-6C)alkyl, fluoro(1-6C)alkyl, difluoro(1-6C)alkyl, trifluoro(1-6C)alkyl, trichloro(1-6C)alkyl, Cyc¹, Ar¹, hetCyc¹ or hetAr¹;

Cyc¹ is (3-6C)cycloalkyl optionally substituted with CF₃;

Ar¹ is phenyl optionally substituted with one or more groups independently selected from halogen, CF₃, (1-4C)alkyl and (1-4C)alkoxy;

hetCyc¹ is a 5-6 membered heterocycle having a ring nitrogen atom and optionally substituted with one or more groups independently selected from halogen, CF₃, (1-4C)alkyl and (1-4C)alkoxy;

hetAr¹ is a 6-membered heteroaryl having a ring nitrogen atom and optionally substituted with one or more groups independently selected from halogen, CF₃, (1-4C)alkyl and (1-4C)alkoxy; and

n is 1, 2 or 3.

In one embodiment, compounds of Formula I include compounds of Formula IB

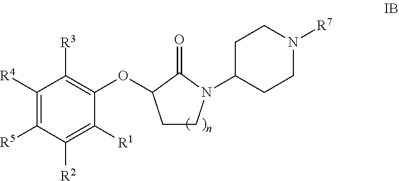

and pharmaceutically acceptable salts thereof, wherein:

R¹, R², R³ and R⁴ are independently selected from H, halogen, CF₃, (1-6C)alkyl and (1-6C)alkoxy;

R⁵ is (1-3C alkyl)sulfonyl, (3-6C cycloalkyl)sulfonyl, (cyclopropylmethyl)sulfonyl, phenylsulfonyl, CN, Br, CF₃, or tetrazolyl optionally substituted with (1-3C)alkyl;

R⁷ is selected from

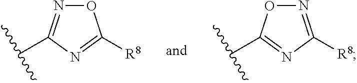

R⁸ is (1-6C)alkyl, fluoro(1-6C)alkyl, difluoro(1-6C)alkyl, trifluoro(1-6C)alkyl, trichloro(1-6C)alkyl, Cyc¹, Ar¹, hetCyc¹ or hetAr¹;

Cyc¹ is (3-6C)cycloalkyl optionally substituted with CF₃;

Ar¹ is phenyl optionally substituted with one or more groups independently selected from halogen, CF₃, (1-4C)alkyl and (1-4C)alkoxy;

hetCyc¹ is a 5-6 membered heterocycle having a ring nitrogen atom and optionally substituted with one or more groups independently selected from halogen, CF₃, (1-4C)alkyl and (1-4C)alkoxy;

hetAr¹ is a 6-membered heteroaryl having a ring nitrogen atom and optionally substituted with one or more groups independently selected from halogen, CF₃, (1-4C)alkyl and (1-4C)alkoxy; and

n is 1, 2 or 3.

In one embodiment of Formula IB, R⁷ is

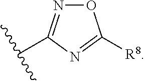

In one embodiment of Formula IB, R⁷ is

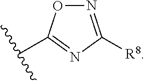

In embodiment, compounds of Formula I include compounds of Formula IC

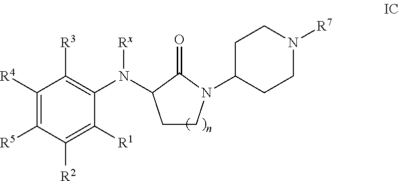

and pharmaceutically acceptable salts thereof, wherein:

R^{x} is H or (1-3C)alkyl;

R¹, R², R³ and R⁴ are independently selected from H, halogen, CF₃, (1-6C)alkyl and (1-6C)alkoxy;

R⁵ is (1-3C alkyl)sulfonyl, (3-6C cycloalkyl)sulfonyl, (cyclopropylmethyl)sulfonyl, phenylsulfonyl, CN, Br, CF₃, or tetrazolyl optionally substituted with (1-3C)alkyl;

R⁷ is selected from

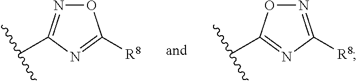

R⁸ is (1-6C)alkyl, fluoro(1-6C)alkyl, difluoro(1-6C)alkyl, trifluoro(1-6C)alkyl, trichloro(1-6C)alkyl, Cyc¹, Ar¹, hetCyc¹ or hetAr¹;

Cyc¹ is (3-6C)cycloalkyl optionally substituted with CF₃;

Ar¹ is phenyl optionally substituted with one or more groups independently selected from halogen, CF₃, (1-4C)alkyl and (1-4C)alkoxy;

hetCyc¹ is a 5-6 membered heterocycle having a ring nitrogen atom and optionally substituted with one or more groups independently selected from halogen, CF₃, (1-4C)alkyl and (1-4C)alkoxy;

hetAr¹ is a 6-membered heteroaryl having a ring nitrogen atom and optionally substituted with one or more groups independently selected from halogen, CF₃, (1-4C)alkyl and (1-4C)alkoxy; and

n is 1, 2 or 3.

In one embodiment of Formula IC, R⁷ is

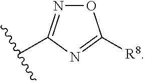

In one embodiment of Formula IC, R⁷ is

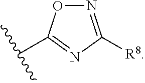

In embodiment, compounds of Formula I include compounds of Formula ID

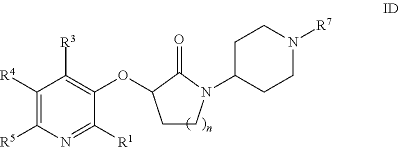

and pharmaceutically acceptable salts thereof, wherein:

R¹, R³ and R⁴ are independently selected from H, halogen, CF₃, (1-6C)alkyl and (1-6C)alkoxy;

R⁵ is (1-3C alkyl)sulfonyl, (3-6C cycloalkyl)sulfonyl, (cyclopropylmethyl)sulfonyl, phenylsulfonyl, CN, Br, CF₃, or tetrazolyl optionally substituted with (1-3C)alkyl;

R⁷ is selected from

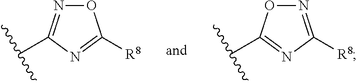

R⁸ is (1-6C)alkyl, fluoro(1-6C)alkyl, difluoro(1-6C)alkyl, trifluoro(1-6C)alkyl, trichloro(1-6C)alkyl, Cyc¹, Ar¹, hetCyc¹ or hetAr¹;

Cyc¹ is (3-6C)cycloalkyl optionally substituted with CF₃;

Ar¹ is phenyl optionally substituted with one or more groups independently selected from halogen, CF₃, (1-4C)alkyl and (1-4C)alkoxy;

hetCyc¹ is a 5-6 membered heterocycle having a ring nitrogen atom and optionally substituted with one or more groups independently selected from halogen, CF₃, (1-4C)alkyl and (1-4C)alkoxy;

hetAr¹ is a 6-membered heteroaryl having a ring nitrogen atom and optionally substituted with one or more groups independently selected from halogen, CF₃, (1-4C)alkyl and (1-4C)alkoxy; and

n is 1, 2 or 3.

In one embodiment of Formula ID, R⁷ is

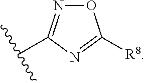

In one embodiment of Formula ID, R⁷ is

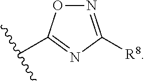

In embodiment, compounds of Formula I include compounds of Formula IE

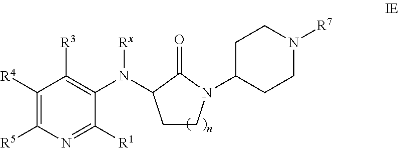

and pharmaceutically acceptable salts thereof, wherein:

R^{x} is H or (1-3C)alkyl;

R¹, R³ and R⁴ are independently selected from H, halogen, CF₃, (1-6C)alkyl and (1-6C)alkoxy;

R⁵ is (1-3C alkyl)sulfonyl, (3-6C cycloalkyl)sulfonyl, (cyclopropylmethyl)sulfonyl, phenylsulfonyl, CN, Br, CF₃, or tetrazolyl optionally substituted with (1-3C)alkyl;

R⁷ is selected from

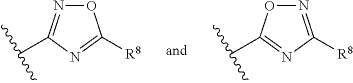

R⁸ is (1-6C)alkyl, fluoro(1-6C)alkyl, difluoro(1-6C)alkyl, trifluoro(1-6C)alkyl, trichloro(1-6C)alkyl, Cyc¹, Ar¹, hetCyc¹ or hetAr¹;

Cyc¹ is (3-6C)cycloalkyl optionally substituted with CF₃;

Ar¹ is phenyl optionally substituted with one or more groups independently selected from halogen, CF₃, (1-4C)alkyl and (1-4C)alkoxy;

hetCyc¹ is a 5-6 membered heterocycle having a ring nitrogen atom and optionally substituted with one or more groups independently selected from halogen, CF₃, (1-4C)alkyl and (1-4C)alkoxy;

hetAr¹ is a 6-membered heteroaryl having a ring nitrogen atom and optionally substituted with one or more groups independently selected from halogen, CF₃, (1-4C)alkyl and (1-4C)alkoxy; and

n is 1, 2 or 3.

In one embodiment of Formula IE, R⁷ is

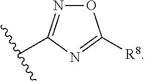

In one embodiment of Formula IE, R⁷ is

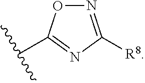

In embodiment, compounds of Formula I include compounds of Formula IF

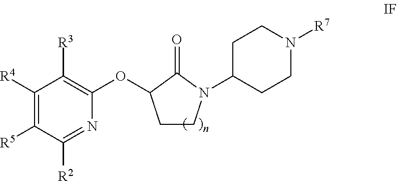

and pharmaceutically acceptable salts thereof, wherein:

R², R³ and R⁴ are independently selected from H, halogen, CF₃, (1-6C)alkyl and (1-6C)alkoxy;

R⁵ is (1-3C alkyl)sulfonyl, (3-6C cycloalkyl)sulfonyl, (cyclopropylmethyl)sulfonyl, phenylsulfonyl, CN, Br, CF₃, or tetrazolyl optionally substituted with (1-3C)alkyl;

R⁷ is selected from

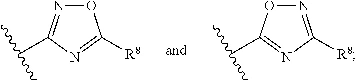

R⁸ is (1-6C)alkyl, fluoro(1-6C)alkyl, difluoro(1-6C)alkyl, trifluoro(1-6C)alkyl, trichloro(1-6C)alkyl, Cyc¹, Ar¹, hetCyc¹ or hetAr¹;

Cyc¹ is (3-6C)cycloalkyl optionally substituted with CF₃;

Ar¹ is phenyl optionally substituted with one or more groups independently selected from halogen, CF₃, (1-4C)alkyl and (1-4C)alkoxy;

hetCyc¹ is a 5-6 membered heterocycle having a ring nitrogen atom and optionally substituted with one or more groups independently selected from halogen, CF₃, (1-4C)alkyl and (1-4C)alkoxy;

hetAr¹ is a 6-membered heteroaryl having a ring nitrogen atom and optionally substituted with one or more groups independently selected from halogen, CF₃, (1-4C)alkyl and (1-4C)alkoxy; and

n is 1, 2 or 3.

In one embodiment of Formula IF, R⁷ is

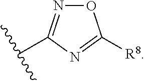

In one embodiment of Formula IF, R⁷ is

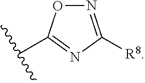

In embodiment, compounds of Formula I include compounds of Formula IG

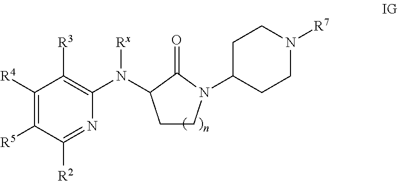

and pharmaceutically acceptable salts thereof, wherein:

R^{x} is H or (1-3C)alkyl;

R², R³ and R⁴ are independently selected from H, halogen, CF₃, (1-6C)alkyl and (1-6C)alkoxy;

R⁵ is (1-3C alkyl)sulfonyl, (3-6C cycloalkyl)sulfonyl, (cyclopropylmethyl)sulfonyl, phenylsulfonyl, CN, Br, CF₃, or tetrazolyl optionally substituted with (1-3C)alkyl;

R⁷ is selected from

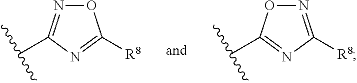

R⁸ is (1-6C)alkyl, fluoro(1-6C)alkyl, difluoro(1-6C)alkyl, trifluoro(1-6C)alkyl, trichloro(1-6C)alkyl, Cyc¹, Ar¹, hetCyc¹ or hetAr¹;

Cyc¹ is (3-6C)cycloalkyl optionally substituted with CF₃;

Ar¹ is phenyl optionally substituted with one or more groups independently selected from halogen, CF₃, (1-4C)alkyl and (1-4C)alkoxy;

hetCyc¹ is a 5-6 membered heterocycle having a ring nitrogen atom and optionally substituted with one or more groups independently selected from halogen, CF₃, (1-4C)alkyl and (1-4C)alkoxy;

hetAr¹ is a 6-membered heteroaryl having a ring nitrogen atom and optionally substituted with one or more groups independently selected from halogen, CF₃, (1-4C)alkyl and (1-4C)alkoxy; and

n is 1, 2 or 3.

In one embodiment of Formula IG, R⁷ is

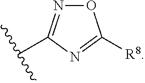

In one embodiment of Formula IG, R⁷ is

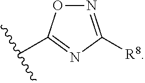

It will be appreciated that certain compounds according to the invention may contain one or more centers of asymmetry and may therefore be prepared and isolated as a mixture of isomers such as a racemic or diastereomeric mixture, or in an enantiomerically or diastereomerically pure form. It is intended that all stereoisomeric forms of the compounds of the invention, including but not limited to, diastereomers, enantiomers and atropisomers, as well as mixtures thereof such as racemic mixtures, form part of the present invention.

It may be advantageous to separate reaction products from one another and/or from starting materials. The desired products of each step or series of steps is separated and/or purified (hereinafter separated) to the desired degree of homogeneity by techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

Enantiomers can be separated by converting the enantiomeric mixture into a diastereomeric mixture by reaction with an appropriate optically active compound (e.g., chiral auxiliary, such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column. Diastereomeric mixtures can be separated into their individual diastereomers on the basis of their physical chemical differences by methods well known to those skilled in the art, such as by chromatography and/or fractional crystallization.

A single stereoisomer, for example, an enantiomer, substantially free of its stereoisomer may be obtained by resolution of the racemic mixture using methods known in the art, such as (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., ed., Drug Stereochemistry: Analytical Methods and Pharmacology. New York: Marcel Dekker, Inc., 1993.

Under method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid, can result in formation of the diastereomeric salts.

Alternatively, by method (2), the substrate to be resolved is reacted with one enantiomer of a chiral compound to form a diastereomeric pair (Eliel, E., and S. Wilen. Stereochemistry of Organic Compounds. New York: John Wiley & Sons, Inc., 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (−) menthyl chloroformate in the presence of base, or Mosher ester, α-methoxy-α-(trifluoromethyl)phenyl acetate (Jacob III, Peyton. “Resolution of (±)-5-Bromonornicotine. Synthesis of (R)- and (S)-Nornicotine of High Enantiomeric Purity.” J. Org. Chem. Vol. 47, No. 21 (1982): pp. 4165-4167), of the racemic mixture, and analyzing the ¹H NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111).

By method (3), a racemic mixture of two enantiomers can be separated by chromatography using a chiral stationary phase (Lough, W. J., ed. Chiral Liquid Chromatography. New York: Chapman and Hall, 1989; Okamoto, Yoshio, et al. “Optical resolution of dihydropyridine enantiomers by high-performance liquid chromatography using phenylcarbamates of polysaccharides as a chiral stationary phase.” J. of Chromatogr. Vol. 513 (1990): pp. 375-378). An example of a chiral stationary phase is a CHIRALPAK ADH column. Enriched or purified enantiomers can be distinguished by methods used to distinguish other chiral molecules with asymmetric carbon atoms, such as optical rotation and circular dichroism.

It will further be appreciated that an enantiomer of a compound of the invention can be prepared by starting with the appropriate chiral starting material.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

Compounds of Formula I include both enantiomers of the position marked with an asterisk (*) as shown below:

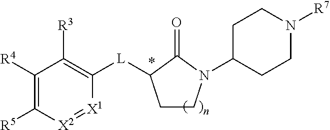

In one embodiment, compound of Formula I have the absolute configuration as shown in Formula I-a

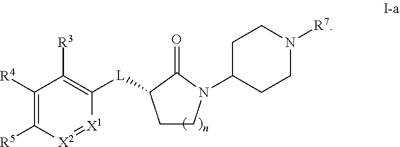

In one embodiment, compound of Formula I have the absolute configuration as shown in Formula I-b:

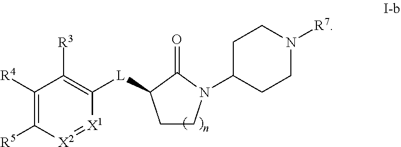

In one embodiment, a compound of Formula I can be enriched in one enantiomer over the other by up to 80% enantiomeric excess. In one embodiment, a compound of Formula I can be enriched in one enantiomer over the other by up to 85% enantiomeric excess. In one embodiment, a compound of Formula I can be enriched in one enantiomer over the other by up to 90% enantiomeric excess. In one embodiment, a compound of Formula I can be enriched in one enantiomer over the other by up to 95% enantiomeric excess.

As used herein, the term “enantiomeric excess” means the absolute difference between the mole fraction of each enantiomer.

The terms “(1-3C)alkyl”, “(1-4C)alkyl” and “(1-6C)alkyl” as used herein refer to saturated linear or branched-chain monovalent hydrocarbon radicals of one to three, one to four, or one to six carbons, respectively. Examples include, but are not limited to, methyl, ethyl, 1-propyl, isopropyl, 1-butyl, isobutyl, sec-butyl, tert-butyl, 2-methyl-2-propyl, pentyl, and hexyl.

The term “fluoro(1-6C)alkyl” as used herein refers to saturated linear or branched-chain monovalent radicals of one to six carbon atoms, wherein one of the hydrogen atoms is replaced by fluorine. Examples include fluoromethyl, fluoromethyl, and 1-fluoropropyl, 2-fluoropropyl.

The term “difluoro(1-6C)alkyl” as used herein refers to saturated linear or branched-chain monovalent radicals of one to six carbon atoms, wherein two of the hydrogen atoms are replaced by fluorine. Examples include difluoromethyl, 2,2-difluoroethyl, and 1,3-difluoroprop-2-yl.

The term “trifluoro(1-6C)alkyl” as used herein refers to saturated linear or branched-chain monovalent radicals of one to six carbon atoms wherein three of the hydrogen atoms are replaced by fluorine. Examples include trifluoromethyl, 2,2,2-trifluoroethyl, and 3,3,3-trifluoropropyl.

The term “trichloro(1-6C)alkyl” as used herein refers to saturated linear or branched-chain monovalent radicals of one to six carbon atoms wherein three of the hydrogen atoms are replaced by chloro. An example includes trichloroethyl.

The terms “(1-4C)alkoxy” and “(1-6C)alkoxy” as used herein refer to saturated linear or branched-chain monovalent alkoxy radicals of one to four or one to six carbon atoms, respectively, wherein the radical is on the oxygen atom. Examples include methoxy, ethoxy, propoxy, isopropoxy, and butoxy.

The term “(1-3C alkyl)sulfonyl” as used herein refers to a (1-3C alkyl)SO₂— group, wherein the radical is on the sulfur atom and the (1-3C alkyl) portion is as defined above. Examples include methylsulfonyl (CH₃SO₂—) and ethylsulfonyl (CH₃SO₂—).

The term “(3-6C cycloalkyl)sulfonyl” as used herein refers to a (3-6C cycloalkyl)SO₂— group, wherein the radical is on the sulfur atom. An example is cyclopropylsulfonyl.

The term “halogen” includes fluoro, chloro, bromo and iodo.

It will also be appreciated that certain compounds of Formula I may be used as intermediates for the preparation of further compounds of Formula I.

The compounds of Formula I include salts thereof. In certain embodiments, the salts are pharmaceutically acceptable salts. In addition, the compounds of Formula I include other salts of such compounds which are not necessarily pharmaceutically acceptable salts, and which may be useful as intermediates for preparing and/or purifying compounds of Formula I and/or for separating enantiomers of compounds of Formula I. Examples of particular salts include trifluoroacetate and hydrochloride salts.

The term “pharmaceutically acceptable” indicates that the substance or composition is compatible chemically and/or toxicologically, with the other ingredients comprising a formulation, and/or the mammal being treated therewith.

It will further be appreciated that the compounds of Formula I and their salts may be isolated in the form of solvates, and accordingly that any such solvate is included within the scope of the present invention.

Compounds of the invention may also contain unnatural proportions of atomic isotopes at one or more of the atoms that constitute such compounds. That is, an atom, in particular when mentioned in relation to a compound according to Formula I, comprises all isotopes and isotopic mixtures of that atom, either naturally occurring or synthetically produced, either with natural abundance or in an isotopically enriched form. For example, when hydrogen is mentioned, it is understood to refer to ¹H, ²H, ³H or mixtures thereof; when carbon is mentioned, it is understood to refer to ¹¹C, ¹²C, ¹³C, ¹⁴C or mixtures thereof; when nitrogen is mentioned, it is understood to refer to ¹³N, ¹⁴N, ¹⁵N or mixtures thereof; when oxygen is mentioned, it is understood to refer to ¹⁴O, ¹⁵O, ¹⁶O, ¹⁷O, ¹⁸O or mixtures thereof; and when fluoro is mentioned, it is understood to refer to ¹⁸F, ¹⁹F or mixtures thereof. The compounds according to the invention therefore also comprise compounds with one or more isotopes of one or more atom, and mixtures thereof, including radioactive compounds, wherein one or more non-radioactive atoms has been replaced by one of its radioactive enriched isotopes. Radiolabeled compounds are useful as therapeutic agents, e.g., cancer therapeutic agents, research reagents, e.g., assay reagents, and diagnostic agents, e.g., in vivo imaging agents. All isotopic variations of the compounds of the present invention, whether radioactive or not, are intended to be encompassed within the scope of the present invention.

The present invention further provides a process for the preparation of a compound of Formula I or a salt thereof as defined herein which comprises:

(a) for a compound of Formula I wherein R⁷ is

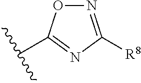

and R⁸ is as defined for Formula I, reacting a corresponding compound having the formula II

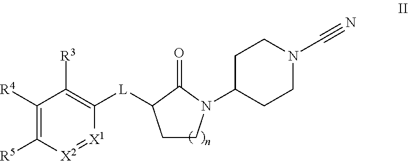

wherein X¹, X², L, R³, R⁴, R⁵ and n are as defined for Formula I, with a reagent having the formula

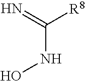

wherein R⁸ is defined for Formula I, in the presence of a Lewis acid; or

(b) for a compound of Formula I wherein R⁷ is

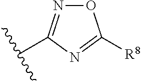

and R⁸ is as defined for Formula I, reacting a corresponding compound having the formula III

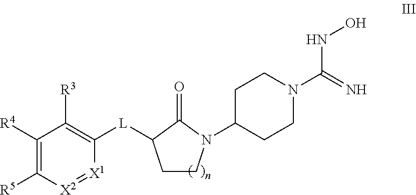

wherein X¹, X², L, R³, R⁴, R⁵ and n are as defined for Formula I, with a reagent having the formula R⁸C(═O)OH or a reactive derivative thereof wherein R⁸ is as defined for Formula I, and optionally in the presence of a base; or

(c) for a compound of Formula I wherein R⁷ is

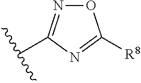

and R⁸ is hetCyc¹, coupling a corresponding compound having the formula IV

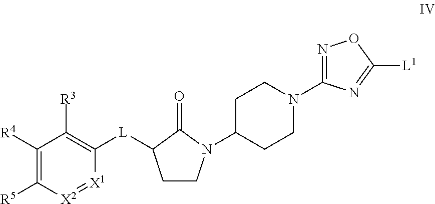

wherein X¹, X², L, R³, R⁴, R⁵ and n are as defined for Formula I and L¹ is a leaving group or atom, with a reagent having the structure:

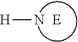

where Ring E is as defined for hetCyc¹, in the presence of a base; and

optionally removing any protecting groups and optionally preparing a salt thereof.

Referring to method (a), suitable Lewis acids include metal halides such as zinc chloride, aluminum chloride, or tin (IV) chloride. Suitable solvents include aprotic solvents such as ethers (for example tetrahydrofuran or p-dioxane). The reaction is conveniently performed at elevated temperatures, for example, between 50 and 150° C., for example 100° C.

Compounds of Formula II can be prepared by reacting a compound of Formula II-A

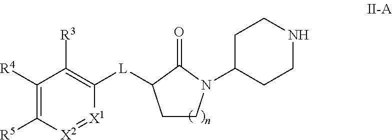

with cyanic bromide (Br—C≡N) in the presence of a base, such as an alkali metal carbonate, such as sodium carbonate, potassium carbonate or cesium carbonate.

Referring to method (b), when reacting a compound of formula III with a carboxylic acid having the formula R⁸C(═O)OH or an acid halide derivative thereof, the reaction is performed in the presence of a base, such as a tertiary amine base such as diisopropylethylamine (DIEA) and triethylamine Suitable solvents include aprotic solvents such as ethers (for example tetrahydrofuran or p-dioxane). When reacting a compound of formula III with an acid anhydride derivative of a compound having the formula R⁸C(═O)OH, the reaction is performed neat, preferably at elevated temperatures, for example at 60 to 120° C., for example at 90° C.

Compounds of Formula III can be prepared by reacting a compound of Formula II with hydroxylamine.

Referring to method (c), the reaction is preferably performed in the presence of an excess amount of the heterocyclic amine represented by the structure:

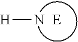

where Ring E is as defined for hetCyc¹. Suitable solvents include alcohols such as ethanol.

Compounds of formula II-A where L is NR^{x} and n is 1 can be prepared as shown in general Scheme 1.

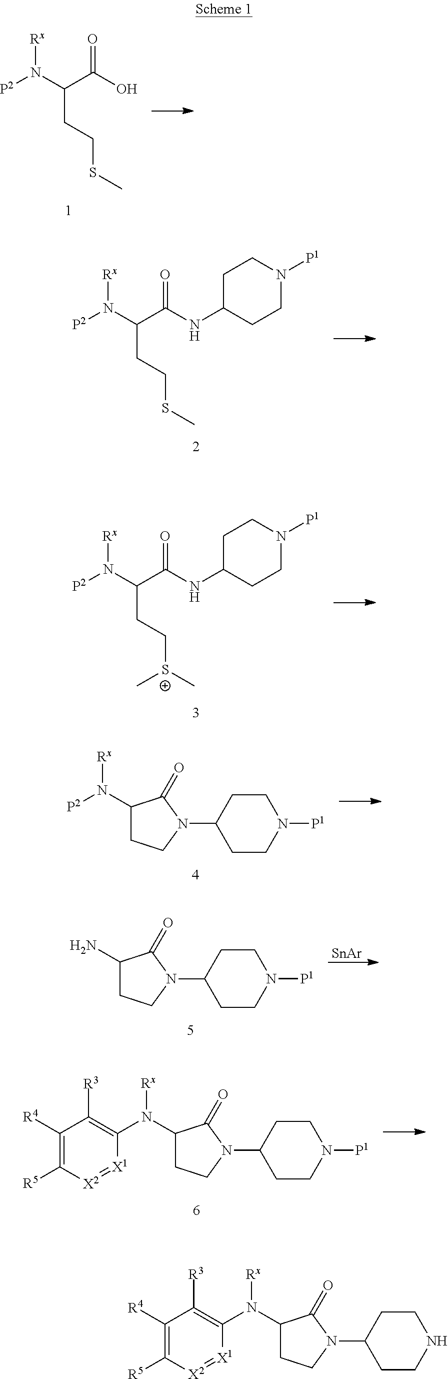

In Scheme 1, P¹ and P² are amine protecting groups. According to Scheme 1, the protected amino piperidine group is coupled to the amino acid intermediate (1) via traditional amide bond forming reagents such as, but not limited to, DCC, to provide compound (2). Compound (2) is activated through methylation reagents such as, but not limited to, methyl iodide to provide compound (3). Cyclization of compound (3) takes place under basic conditions such as, but not limited to, NaH or LHMDS to afford compound (4). Removal of the nitrogen protecting group P² of compound (4) under standard deprotection conditions to provide compound (5), followed by a SnAr reaction with an appropriately functionalized aryl or heteroaryl group provides compounds of formula II-A after removal of the protecting group P¹ of compound (6) under standard deprotection conditions.

In one embodiment, compounds of formula II-A where L is O and n is 1, 2 or 3 can be prepared as shown in Scheme 2.

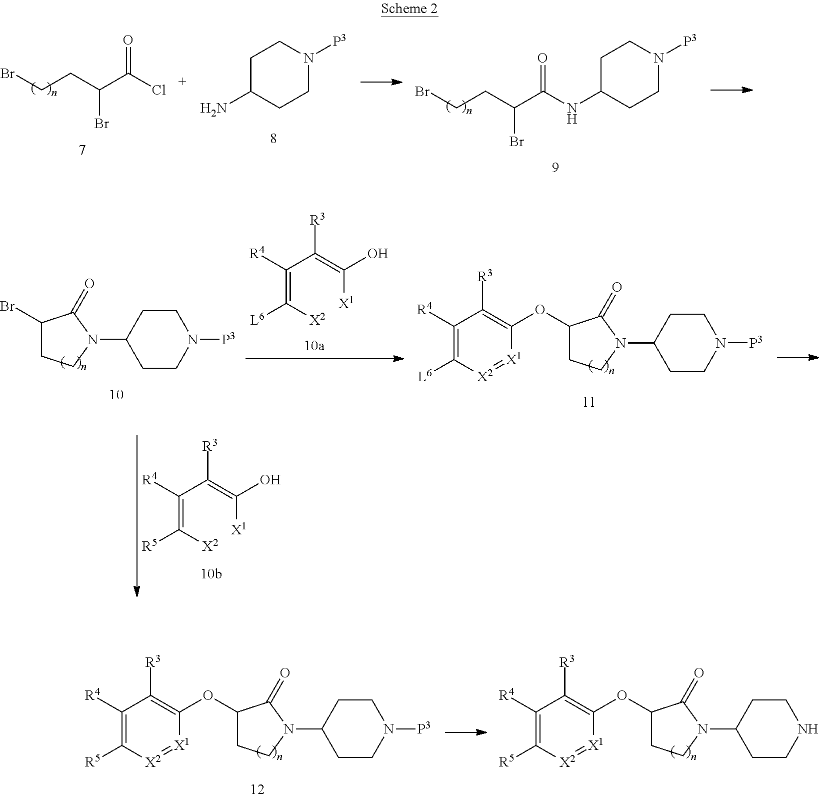

In Scheme 2, P³ is an amine protecting group. According to Scheme 2, acylation of the amino piperidine (8) with acid chloride (7) affords the compound (9). Cyclization of compound (9) to form the lactam (10) is promoted by bases such as, but not limited to, alkali metal hydrides such as NaH, alkali metal amine bases such as lithium diisopropylamide, or silicon-containing alkali metal amides (e.g., sodium hexamethyldisilazide or lithium hexamethyldisilazide). Compound (10) can be coupled with compound (10a) (where L⁶ is a leaving group or atom) under basic conditions, for example, in the presence of an alkali metal hydride or carbonate, such as sodium hydride, potassium hydride, sodium carbonate, potassium carbonate or cesium carbonate. When R⁵ is a group having the R⁵SO₂— where R⁵ is (1-3C) alkyl, (3-6C)cycloalkyl, cyclopropylmethyl- or phenyl, compound (11) can be coupled with a corresponding compound having the formula R⁵SO₂Na in the presence of a metal catalyst such as, but not limited to, copper and palladium catalysts, to provide compound (12). Alternatively, when R⁵ is CN, compound (11) can be reacted with CuCN to provide compound (12). Alternatively, compound (10) can be coupled with compound (10b) to provide compound (12). Removal of the protecting group P³ of compound (12) under standard deprotection conditions affords compounds of formula II-A.

In one embodiment, compounds of formula II-A where L is NR^{x} and n is 2 or 3 can be prepared as shown in Scheme 3.

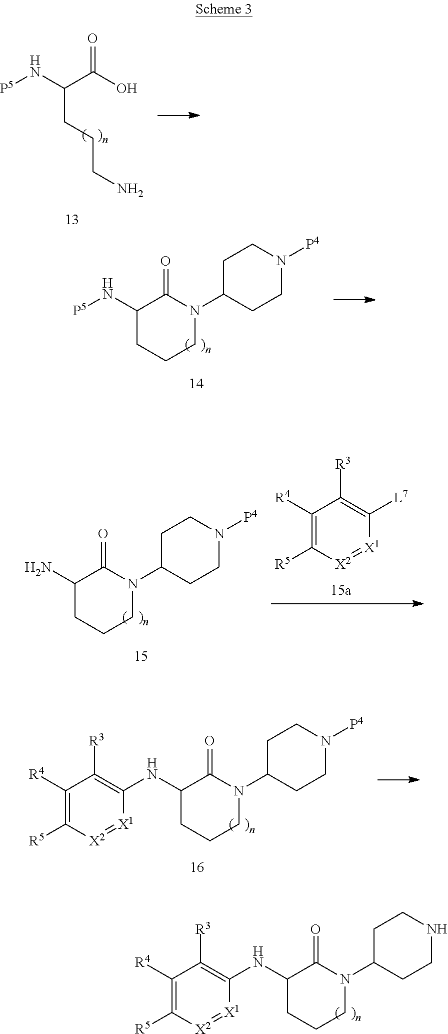

In Scheme 3, P⁴ and P⁵ are amine protecting groups. According to Scheme 3, amino acid (13) is converted to lactam (14) through sequential reductive amination and amide bond formation. Removal of protecting group P⁵ of compound (14) under standard deprotection conditions, followed by coupling of the deprotected compound (15) with compound (15a) under standard SnAr conditions affords intermediate (16). The NH₂ group of compound (15) can optionally be alkylated under standard alkylation conditions known to persons skilled in the art prior to removal of the protecting group P⁴. Removal of the protecting group P⁴ of compound (16) affords compounds of formula II-A.

Amine groups in compounds described in any of the above methods may be protected with any convenient amine protecting group, for example as described in Greene & Wuts, eds., “Protecting Groups in Organic Synthesis”, 2^{nd} ed. New York; John Wiley & Sons, Inc., 1991. Examples of amine protecting groups include acyl and alkoxycarbonyl groups, such as t-butoxycarbonyl (BOC), and [2-(trimethylsilyl)ethoxy]methyl (SEM). Likewise, carboxyl groups may be protected with any convenient carboxyl protecting group, for example as described in Greene & Wuts, eds., “Protecting Groups in Organic Synthesis”, 2^{nd} ed. New York; John Wiley & Sons, Inc., 1991. Examples of carboxyl protecting groups include (1-6C)alkyl groups, such as methyl, ethyl and t-butyl. Alcohol groups may be protected with any convenient alcohol protecting group, for example as described in Greene & Wuts, eds., “Protecting Groups in Organic Synthesis”, 2^{nd} ed. New York; John Wiley & Sons, Inc., 1991. Examples of alcohol (hydroxyl) protecting groups include benzyl, trityl, silyl ethers, and the like.

The compounds of the formulas II, II-A, III, and IV are also believed to be novel and are provided as further aspects of the invention.

Compounds of Formula I are modulators of GPR119 and are useful for treating or preventing disease including, but not limited to, type 2 diabetes, diabetic complications, symptoms of diabetes, metabolic syndrome, obesity, dyslipidemia, and related conditions.

The ability of compounds of the invention to act as modulators of GPR119 may be demonstrated by the assay described in Example A.

The term “modulate” refers to the treating, prevention, suppression, enhancement or induction of a function or condition. For example, compounds can modulate Type 2 diabetes by increasing insulin in a human, thereby suppressing hyperglycemia.

The term “modulator” as used herein includes the terms agonist, antagonist, inverse agonist, and partial agonist.

The term “agonist” refers to a compound that binds to a receptor and triggers a response in a cell. An agonist mimics the effect of an endogenous ligand, a hormone for example, and produces a physiological response similar to that produced by the endogenous ligand.

The term “partial agonist” refers to a compound that binds to a receptor and triggers a partial response in a cell. A partial agonist produces only a partial physiological response of the endogenous ligand.

The term “antagonist” as used herein refers to is a type of receptor ligand or drug that does not provoke a biological response itself upon binding to a receptor, but blocks or dampens agonist-mediated responses.

The term “inverse agonist” as used herein refers to an agent that binds to the same receptor binding-site as an agonist for that receptor and reverses constitutive activity of the receptor.

Certain compounds of Formula I are agonists of GPR119.

Certain compounds of Formula I are inverse agonists of GPR119.

Certain compounds of Formula I are antagonists of GPR119.

In certain embodiments, compound of Formula I are useful for treating or preventing type 2 diabetes mellitus (also known as non-insulin dependent diabetes mellitus, or T2DM). Diabetes mellitus is a condition where the fasting plasma glucose level (glucose concentration in venous plasma) is greater than or equal to 126 mg/dL (tested on two occasions) and the 2-hour plasma glucose level of a 75 g oral glucose tolerance test (OGTT) is greater than or equal to 200 mg/dL. Additional classic symptoms include polydipsia, polyphagia and polyuria.

Accordingly, one aspect of the present invention provides methods for treating or preventing type 2 diabetes mellitus in a mammal, comprising administering to the mammal in need of such treatment a therapeutically effective amount of a compound of Formula I or a pharmaceutically acceptable salt thereof.

In certain embodiments, compound of Formula I are useful for treating or preventing diabetic complications. The term “diabetic complications” includes, but is not limited to, microvascular complications and macrovascular complications. Microvascular complications are those complications that generally result in small blood vessel damage. These complications include, for example, retinopathy (the impairment or loss of vision due to blood vessel damage in the eyes); neuropathy (nerve damage and foot problems due to blood vessel damage to the nervous system); and nephropathy (kidney disease due to blood vessel damage in the kidneys). Macrovascular complications are those complications that generally result from large blood vessel damage. These complications include, e.g., cardiovascular disease and peripheral vascular disease. Cardiovascular disease is generally one of several forms, including, e.g., hypertension (also referred to as high blood pressure), coronary heart disease, stroke, and rheumatic heart disease. Peripheral vascular disease refers to diseases of any of the blood vessels outside of the heart. It is often a narrowing of the blood vessels that carry blood to leg and arm muscles.

Accordingly, one aspect of the present invention provides methods for treating or preventing diabetic complications in a mammal, comprising administering to the mammal in need of such treatment a therapeutically effective amount of a compound of Formula I or a pharmaceutically acceptable salt thereof. In one embodiment, the diabetic complication is retinopathy (also known as diabetic retinopathy).

In certain embodiments, compound of Formula I are useful for treating or preventing symptoms of diabetes. The term “symptom” of diabetes, includes, but is not limited to, polyuria, polydipsia, and polyphagia, as used herein, incorporating their common usage. For example, “polyuria” means the passage of a large volume of urine during a given period; “polydipsia” means chronic, excessive thirst; and “polyphagia” means excessive eating. Other symptoms of diabetes include, e.g., increased susceptibility to certain infections (especially fungal and staphylococcal infections), nausea, and ketoacidosis (enhanced production of ketone bodies in the blood).

Accordingly, one aspect of the present invention provides methods for treating or preventing symptoms of diabetes in a mammal, comprising administering to the mammal in need of such treatment a therapeutically effective amount of a compound of Formula I or a pharmaceutically acceptable salt thereof.

In certain embodiments, compound of Formula I are useful for treating or preventing metabolic syndrome in a mammal. The term “metabolic syndrome” refers to a cluster of metabolic abnormalities including abdominal obesity, insulin resistance, glucose intolerance, hypertension and dyslipidemia. These abnormalities are known to be associated with an increased risk of type 2 diabetes and cardiovascular disease. Compounds of Formula I are also useful for reducing the risks of adverse sequelae associated with metabolic syndrome, and in reducing the risk of developing atherosclerosis, delaying the onset of atherosclerosis, and/or reducing the risk of sequelae of atherosclerosis. Sequelae of atherosclerosis include angina, claudication, heart attack, stroke, and others.

Accordingly, one aspect of the present invention provides methods of treating a metabolic syndrome in a mammal, comprising administering to the mammal in need of such treatment a therapeutically effective amount of a compound of Formula I or a pharmaceutically acceptable salt thereof. In one embodiment, the metabolic syndrome is hyperglycemia. In one embodiment, the metabolic syndrome is impaired glucose tolerance.

In one embodiment, the metabolic syndrome is insulin resistance. In one embodiment, the metabolic syndrome is atherosclerosis.

In certain embodiments, compound of Formula I are useful for treating or preventing obesity in a mammal. The term “obesity” refers to, according to the World Health Organization, a Body Mass Index (“BMI”) greater than 27.8 kg/m² for men and 27.3 kg/m² for women (BMI equals weight (kg)/height (m²)). Obesity is linked to a variety of medical conditions including diabetes and hyperlipidemia. Obesity is also a known risk factor for the development of Type 2 diabetes.

Accordingly, one aspect of the present invention provides methods of treating or preventing obesity in a mammal, comprising administering to the mammal in need of such treatment a therapeutically effective amount of a compound of Formula I or a pharmaceutically acceptable salt thereof.

Compounds of Formula I may also be useful for treating or preventing diseases and disorders such as, but not limited to, dyslipidemia and dyslipoproteinemia.

The term “dyslipidemia” refers to abnormal levels of lipoproteins in blood plasma including both depressed and/or elevated levels of lipoproteins (e.g., elevated levels of LDL and/or VLDL, and depressed levels of HDL).

The term “dyslipoproteinemia” refers to abnormal lipoproteins in the blood, including hyperlipidemia, hyperlipoproteinemia (excess of lipoproteins in the blood) including type I, II-a (hypercholesterolemia), II-b, III, IV (hypertriglyceridemia) and V (hypertriglyceridemia).

Accordingly, one aspect of the present invention provides methods of treating or preventing dyslipidemia in a mammal, comprising administering to the mammal in need of such treatment a therapeutically effective amount of a compound of Formula I or a pharmaceutically acceptable salt thereof.

Another aspect of the present invention provides methods of treating or preventing dyslipoproteinemia in a mammal, comprising administering to the mammal in need of such treatment a therapeutically effective amount of a compound of Formula I or a pharmaceutically acceptable salt thereof.

By elevating levels of active GLP-1 in vivo, the compounds are useful in treating neurological disorders such as Alzheimer's disease, multiple sclerosis, and schizophrenia.

Accordingly, one aspect of the invention provides methods of treating neurological disorders in a mammal, comprising administering to the mammal in need thereof a therapeutically effective amount of a compound of Formula I or a pharmaceutically acceptable salt thereof. In one embodiment, the neurological disorder is Alzheimer's disease.

Compounds of Formula I generally are useful for treating or preventing diseases and conditions selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis.

Accordingly, one aspect of the invention provides methods for treating or preventing diseases and conditions selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis, comprising administering to the mammal in need of such treatment a therapeutically effective amount of a compound of Formula I or a pharmaceutically acceptable salt thereof. In one embodiment, the disease is selected from type 2 diabetes.

According to another aspect, the invention provides methods for treating or preventing diseases and conditions selected from type 2 diabetes, symptoms of diabetes, diabetic complications, metabolic syndrome (including hyperglycemia, impaired glucose tolerance, and insulin resistance), obesity, dyslipidemia and dyslipoproteinemia.

Compounds of Formula I may also be useful for increasing satiety, reducing appetite, and reducing body weight in obese subjects and may therefore be useful in reducing the risk of co-morbidities associated with obesity such as hypertension, atherosclerosis, diabetes, and dyslipidemia.

Accordingly, the present invention provides methods of inducing satiety, reducing appetite, and reducing body weight in a mammal, comprising administering to the mammal in need thereof a therapeutically effective amount of a compound of Formula I or a pharmaceutically acceptable salt thereof.

In one aspect, the present invention provides methods of inducing satiety in a mammal, comprising administering to the mammal in need thereof a therapeutically effective amount of a compound of Formula I or a pharmaceutically acceptable salt thereof.

In one aspect, the present invention provides methods of decreasing food intake in a mammal, comprising administering to the mammal in need thereof a therapeutically effective amount of a compound of Formula I or a pharmaceutically acceptable salt thereof.

In one aspect, the present invention provides methods of controlling or decreasing weight gain of a mammal, comprising administering to the mammal in need thereof a therapeutically effective amount of a compound of Formula I or a pharmaceutically acceptable salt thereof.

Compounds of Formula I may be administered alone as a sole therapy or can be administered in addition with one or more other substances and/or treatments that work by the same or a different mechanism of action. These agents may be administered with one or more compounds of Formula I as part of the same or separate dosage forms, via the same or different routes of administration, and on the same or different administration schedules according to standard pharmaceutical practice known to one skilled in the art.

Accordingly, compounds of Formula I can be used in combination with a therapeutically effective amount of one or more additional drugs such as insulin preparations, agents for improving insulin resistance (for example PPAR gamma agonists), alpha-glucosidase inhibitors, biguanides (e.g., metformin), insulin secretagogues, dipeptidylpeptidase IV (DPP4) inhibitors (e.g., sitagliptin), beta-3 agonists, amylin agonists, phosphotyrosine phosphatase inhibitors, gluconeogenesis inhibitors, sodium-glucose cotransporter inhibitors, known therapeutic agents for diabetic complications, antihyperlipidemic agents, hypotensive agents, antiobesity agents, GLP-I, GIP-I, GLP-I analogs such as exendins, (for example exenatide (Byetta), exenatide-LAR, and liraglutide), and hydroxysterol dehydrogenase-1 (HSD-I) inhibitors. In one embodiment, a compound of Formula I is used in combination with a biguanide. In one embodiment, a compound of Formula I is used in combination with metformin. In one embodiment, a compound of Formula I is used in combination with metformin for the treatment of type 2 diabetes. In one embodiment, a compound as described in any one of Examples 1-67 is used in combination with metformin for the treatment of type 2 diabetes. In one embodiment, a compound of Formula I is used in combination with a DPP4 inhibitor. In one embodiment, a compound of Formula I is used in combination with sitagliptin. In one embodiment, a compound of Formula I is used in combination with sitagliptin for the treatment of type 2 diabetes. In one embodiment, a compound of any one of Examples 1-67 is used in combination with sitagliptin for the treatment of type 2 diabetes.

Accordingly, there is provided a method of treating a disease or condition selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis in a mammal, which comprises administering to said mammal a therapeutically effective amount of a compound of Formula I or a pharmaceutically acceptable salt thereof, in combination with in combination with a therapeutically effective amount of one or more additional drugs. In one embodiment, the combination is administered for the treatment of type 2 diabetes. In one embodiment, the additional drug is a biguanide. In one embodiment, the additional drug is metformin. In one embodiment, the additional drug is a DPP4 inhibitor. In one embodiment, the additional drug is sitagliptin.

As used herein, terms “treat” or “treatment” refer to therapeutic, prophylactic, palliative or preventative measures. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder, as well as those prone to have the condition or disorder or those in which the condition or disorder is to be prevented.

In one embodiment, the terms “treatment” or “treating” as used herein, mean an alleviation, in whole or in part, of symptoms associated with a disorder or condition as described herein, or slowing, or halting of further progression or worsening of those symptoms.

In one embodiment, the term “preventing” as used herein means the prevention of the onset, recurrence or spread, in whole or in part, of the disease or condition as described herein, or a symptom thereof.

The terms “effective amount” and “therapeutically effective amount” refer to an amount of compound that, when administered to a mammal in need of such treatment, is sufficient to (i) treat or prevent a particular disease, condition, or disorder, (ii) attenuate, ameliorate, or eliminate one or more symptoms of the particular disease, condition, or disorder, or (iii) prevent or delay the onset of one or more symptoms of the particular disease, condition, or disorder described herein. The amount of a compound of Formula I that will correspond to such an amount will vary depending upon factors such as the particular compound, disease condition and its severity, the identity (e.g., weight) of the mammal in need of treatment, but can nevertheless be routinely determined by one skilled in the art.

As used herein, the term “mammal” refers to a warm-blooded animal that has or is at risk of developing a disease described herein and includes, but is not limited to, guinea pigs, dogs, cats, rats, mice, hamsters, and primates, including humans.

Compounds of the invention may be administered by any convenient route, e.g. into the gastrointestinal tract (e.g. rectally or orally), the nose, lungs, musculature or vasculature, or transdermally or dermally. Compounds may be administered in any convenient administrative form, for example tablets, powders, capsules, solutions, dispersions, suspensions, syrups, sprays, suppositories, gels, emulsions, patches etc. Such compositions may contain components conventional in pharmaceutical preparations, e.g. diluents, carriers, pH modifiers, sweeteners, bulking agents, excipients and further active agents. If parenteral administration is desired, the compositions will be sterile and in a solution or suspension form suitable for injection or infusion. Such compositions form a further aspect of the invention.

The present invention further provides a pharmaceutical composition, which comprises a compound of Formula I or a pharmaceutically acceptable salt thereof, as defined hereinabove, and a pharmaceutically acceptable carrier, diluent or excipient.

An example of a suitable oral dosage form is a tablet containing about 25 mg, 50 mg, 100 mg, 250 mg, or 500 mg of the compound of the invention compounded with about 90-30 mg anhydrous lactose, about 5-40 mg sodium croscarmellose, about 5-30 mg polyvinylpyrrolidone (“PVP”) K30, and about 1-10 mg magnesium stearate. The powdered ingredients are first mixed together and then mixed with a solution of the PVP. The resulting composition can be dried, granulated, mixed with the magnesium stearate and compressed to tablet form using conventional equipment. An aerosol formulation can be prepared by dissolving the compound, for example 5-400 mg, of the invention in a suitable buffer solution, e.g. a phosphate buffer, adding a tonicifier, e.g., a salt such sodium chloride, if desired. The solution is typically filtered, e.g., using a 0.2 micron filter, to remove impurities and contaminants.

The present invention further provides a compound of Formula I or a pharmaceutically acceptable salt thereof, for use in therapy. In one embodiment, the present invention provides a compound of Formula I or a pharmaceutically acceptable salt thereof, for use in treating a disease or disorder selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis. In one embodiment, the present invention provides a compound of Formula I or a pharmaceutically acceptable salt thereof, for use in the treatment of type 2 diabetes, symptoms of diabetes, diabetic complications, metabolic syndrome (including hyperglycemia, impaired glucose tolerance, and insulin resistance), obesity, dyslipidemia, or dyslipoproteinemia. In one embodiment, the present invention provides a compound of Formula I or a pharmaceutically acceptable salt thereof, for use in the treatment of type 2 diabetes.

In one embodiment, the invention provides a compound of Formula I or a pharmaceutically acceptable salt thereof, for use in the treatment of a disease or disorder selected from type 2 diabetes, symptoms of diabetes, diabetic complications, metabolic syndrome (including hyperglycemia, impaired glucose tolerance, and insulin resistance), obesity, dyslipidemia, and dyslipoproteinemia.

In one embodiment, the invention provides a compound of Formula I or a pharmaceutically acceptable salt thereof, for use in the treatment of type 2 diabetes mellitus in a mammal.

In one embodiment, the invention provides a compound of Formula I or a pharmaceutically acceptable salt thereof, for use in the treatment of diabetic complications in a mammal.

In one embodiment, the invention provides a compound of Formula I or a pharmaceutically acceptable salt thereof, for use in the treatment of symptoms of diabetes in a mammal.

In one embodiment, the invention provides a compound of Formula I or a pharmaceutically acceptable salt thereof, for use in the treatment of metabolic syndrome in a mammal. In one embodiment, the metabolic syndrome is hyperglycemia. In one embodiment, the metabolic syndrome is impaired glucose tolerance. In one embodiment, the metabolic syndrome is insulin resistance. In one embodiment, the metabolic syndrome is atherosclerosis.

In one embodiment, the invention provides a compound of Formula I or a pharmaceutically acceptable salt thereof, for use in the treatment of obesity in a mammal.

In one embodiment, the invention provides a compound of Formula I or a pharmaceutically acceptable salt thereof, for use in the treatment of dyslipidemia in a mammal.

In one embodiment, the invention provides a compound of Formula I or a pharmaceutically acceptable salt thereof, for use in the treatment of dyslipoproteinemia in a mammal.

In one embodiment, the invention provides a compound of Formula I or a pharmaceutically acceptable salt thereof, for use in the treatment of neurological disorders in a mammal. In one embodiment, the neurological disorder is Alzheimer's disease.

In one embodiment, the invention provides a compound of Formula I or a pharmaceutically acceptable salt thereof, for use in inducing satiety in a mammal.

In one embodiment, the invention provides a compound of Formula I or a pharmaceutically acceptable salt thereof, for use in decreasing food intake in a mammal.

In one embodiment, the invention provides a compound of Formula I or a pharmaceutically acceptable salt thereof, for use in controlling or decreasing weight gain in a mammal.

According to a further aspect, the present invention provides the use of a compound of Formula I or a pharmaceutically acceptable salt thereof, in the treatment of a disease or condition selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis.

According to a further aspect, the present invention provides the use of a compound of Formula I or a pharmaceutically acceptable salt thereof, in the treatment of a disease or condition selected from type 2 diabetes, symptoms of diabetes, diabetic complications, metabolic syndrome (including hyperglycemia, impaired glucose tolerance, and insulin resistance), obesity, dyslipidemia, and dyslipoproteinemia,

According to a further aspect, the present invention provides the use of a compound of Formula I or a pharmaceutically acceptable salt thereof, in the treatment of type 2 diabetes mellitus in a mammal.

According to a further aspect, the present invention provides the use of a compound of Formula I or a pharmaceutically acceptable salt thereof, in the treatment of diabetic complications in a mammal.

According to a further aspect, the present invention provides the use of a compound of Formula I or a pharmaceutically acceptable salt thereof, in the treatment of symptoms of diabetes in a mammal.

According to a further aspect, the present invention provides the use of a compound of Formula I or a pharmaceutically acceptable salt thereof, in the treatment of metabolic syndrome in a mammal.

According to a further aspect, the present invention provides the use of a compound of Formula I or a pharmaceutically acceptable salt thereof, in the treatment of metabolic syndrome in a mammal. In one embodiment, the metabolic syndrome is hyperglycemia. In one embodiment, the metabolic syndrome is impaired glucose tolerance.

In one embodiment, the metabolic syndrome is insulin resistance. In one embodiment, the metabolic syndrome is atherosclerosis.

According to a further aspect, the present invention provides the use of a compound of Formula I or a pharmaceutically acceptable salt thereof, in the treatment of obesity in a mammal.

According to a further aspect, the present invention provides the use of a compound of Formula I or a pharmaceutically acceptable salt thereof, in the treatment of dyslipidemia in a mammal.

According to a further aspect, the present invention provides the use of a compound of Formula I or a pharmaceutically acceptable salt thereof, in the treatment of dyslipoproteinemia in a mammal.

According to a further aspect, the present invention provides the use of a compound of Formula I or a pharmaceutically acceptable salt thereof, in the treatment of neurological disorders in a mammal. In one embodiment, the neurological disorder is Alzheimer's disease.

According to a further aspect, the present invention provides the use of a compound of Formula I or a pharmaceutically acceptable salt thereof, in inducing satiety in a mammal.

According to a further aspect, the present invention provides the use of a compound of Formula I or a pharmaceutically acceptable salt thereof, in decreasing food intake in a mammal.

According to a further aspect, the present invention provides the use of a compound of Formula I or a pharmaceutically acceptable salt thereof, in controlling or decreasing weight gain in a mammal.

Another embodiment of the present invention provides the use of a compound of Formula I, or a pharmaceutically acceptable salt thereof, in the manufacture of a medicament for treating or preventing type 2 diabetes mellitus in a mammal.

Another embodiment of the present invention provides the use of a compound of Formula I, or a pharmaceutically acceptable salt thereof, in the manufacture of a medicament for treating or preventing diabetic complications.

Another embodiment of the present invention provides the use of a compound of Formula I, or a pharmaceutically acceptable salt thereof, in the manufacture of a medicament for treating or preventing symptoms of diabetes.

Another embodiment of the present invention provides the use of a compound of Formula I, or a pharmaceutically acceptable salt thereof, in the manufacture of a medicament for treating or preventing metabolic syndrome in a mammal.

Another embodiment of the present invention provides the use of a compound of Formula I, or a pharmaceutically acceptable salt thereof, in the manufacture of a medicament for treating or preventing obesity in a mammal.

Another embodiment of the present invention provides the use of a compound of Formula I, or a pharmaceutically acceptable salt thereof, in the manufacture of a medicament for treating or preventing dyslipidemia or dyslipoproteinemia.

Another embodiment of the present invention provides the use of a compound of Formula I, or a pharmaceutically acceptable salt thereof, in the manufacture of a medicament for treating neurological disorders in a mammal.

Another embodiment of the present invention provides the use of a compound of Formula I, or a pharmaceutically acceptable salt thereof, in the manufacture of a medicament for inducing satiety in a mammal.

Another embodiment of the present invention provides the use of a compound of Formula I, or a pharmaceutically acceptable salt thereof, in the manufacture of a medicament for decreasing food intake in a mammal.

Another embodiment of the present invention provides the use of a compound of Formula I, or a pharmaceutically acceptable salt thereof, in the manufacture of a medicament for controlling or decreasing weight gain of a mammal.

In one embodiment, the compound of Formula I is selected from any one of the compounds of Examples 1-67 or a pharmaceutically acceptable salt thereof. In one embodiment, the pharmaceutically acceptable salt is a trifluoroacetate and hydrochloride salts.

### EXAMPLES

The following examples illustrate the invention. In the examples described below, unless otherwise indicated all temperatures are set forth in degrees Celsius. Reagents were purchased from commercial suppliers such as Aldrich Chemical Company, Lancaster, Alfa, Aesar, TCI, Maybridge, or other suitable suppliers, and were used without further purification unless otherwise indicated. THF, DCM, toluene, DMF and dioxane were purchased from commercial vendors and used as received.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried or dried under a stream of dry nitrogen.

Column chromatography was done on a Biotage system (Manufacturer: Dyax Corporation) having a silica gel or C-18 reverse phase column, or on a silica SepPak cartridge (Waters), or using conventional flash column chromatography on silica gel, unless otherwise specified.

Abbreviations used herein have the following meanings:

| | |
|---|---|
| ACN | Acetonitrile |
| APCI | Atmospheric Pressure Chemical Ionization |
| Boc | tert-butoxycarbonyl |
| CDCl₃ | Deuterated Chloroform |
| DCC | N,N′-dicyclohexylcarbodiimide |
| DCM | Dichloromethane |
| DIEA | Diisopropylethylamine |
| DMF | N,N-Dimethylformamide |
| DMSO | Dimethylsulfoxide |
| HPLC | High performance liquid chromatography |
| NaBH(OAc)₃ | Sodium triacetoxyborohydride |
| Pd₂dba₃ | Tris(dibenzylideneacetone)dipalladium(0) |
| TFA | Trifluoroacetic acid |
| THF | Tetrahydrofuran |

### Biological Assay

### Example A

### cAMP Production Assay

The assay utilized HEK-293 cells that stably express a modified version of the GPR119 receptor (94% identity to human receptor), under the control of a CMV promoter containing a tet-on element for tetracycline-inducible expression. GPR119 agonist-induced cyclic AMP (cAMP) production was measured in this cell line using the LANCE cAMP kit (Perkin Elmer, Waltham, Mass.). To generate a working stock of cells for the assay, cells were treated overnight with 1 μg/mL doxycycline at 37° C. in the presence of 5% CO₂ to induce receptor expression. Cells were then harvested by enzymatic dissociation with 0.05% trypsin, resuspended in freezing medium (DMEM growth medium with 10% each of fetal bovine serum and DMSO), aliquoted and frozen at −80° C. On the day of the assay, frozen cells were thawed, washed 1× in PBS and resuspended in Hank's buffered salt solution (HBSS) containing 5 mM HEPES, 0.1% BSA and Alexa Fluor 647-conjugated anti-cAMP antibody (diluted 1:100). The cell suspension was then transferred to a Proxiplate Plus white 384-well assay plate (Perkin-Elmer) at 2000 cells/well. Test compounds at final concentrations ranging from 0.2 nM to 10 μM were added to the assay plate, followed by a one-hour incubation at ambient temperature (volume=10 μL/well). DMSO concentration was held constant at 0.5%. After incubation with test compounds, 10 μL of a detergent buffer containing a biotinylated cAMP/Europium-conjugated streptavidin complex (Europium-labeled cAMP tracer) were added to each well on the assay plate, followed by a 2-hour incubation at ambient temperature. During this incubation cAMP released from lysed cells competes with the Europium-labeled cAMP tracer for binding to the Alexa Fluor 647-conjugated antibody. Agonist-induced cellular cAMP production resulted in increased competition with the Europium-labeled cAMP tracer, leading to a proportional decrease in the time-resolved fluorescence resonance energy transfer (TR-FRET) signal detected by the Perkin-Elmer Envision plate reader. Cellular cAMP levels were then determined by interpolation of raw signal data using a cAMP standard curve. Compounds were determined to have agonist activity if they stimulated a 1.5-fold or greater increase in cAMP relative to basal levels. Results for the compounds of Examples 1-67 are shown in Table A.

TABLE A

| | cAMP production in HEK-293 cells | |
|---|---|---|
| Ex. # | (nMol) | Fold over baseline |
| 1 | 25.0 | 4.6 |
| 2 | 27.4 | 5.6 |
| 3 | 21.7 | 3.3 |
| 4 | 18.7 | 3.9 |
| 5 | 22.7 | 4.8 |
| 6 | 22.5 | 4.9 |
| 7 | 27.8 | 5.7 |
| 8 | 25.0 | 4.9 |
| 9 | 11.4 | 3.5 |
| 10 | 8.3 | 2.9 |
| 11 | 13.5 | 4.0 |
| 12 | 16.1 | 3.4 |
| 13 | 17.3 | 3.0 |
| 14 | 18.9 | 3.5 |
| 15 | 20.8 | 3.8 |
| 16 | 17.0 | 4.3 |
| 17 | 28.3 | 4.8 |
| 18 | 19.4 | 3.7 |
| 19 | 24.9 | 4.1 |
| 20 | 19.4 | 3.7 |
| 21 | 21.0 | 2.8 |
| 22 | 17.6 | 3.6 |
| 23 | 19.2 | 3.6 |
| 24 | 81.6 | 4.4 |
| 25 | 49.0 | 2.9 |
| 26 | 5.1 | 2.0 |
| 27 | 98.9 | 5.1 |
| 28 | 101.5 | 5.0 |
| 29 | 47.6 | 3.0 |
| 30 | 7.5 | 3.1 |
| 31 | 4.9 | 2.3 |
| 32 | 7.9 | 2.4 |
| 33 | 10.1 | 2.5 |
| 34 | 7.3 | 2.8 |
| 35 | 13.1 | 3.9 |
| 36 | 9.9 | 4.3 |
| 37 | 12.2 | 3.7 |
| 38 | 9.0 | 3.5 |
| 39 | 8.7 | 2.9 |
| 40 | 17.5 | 5.7 |
| 41 | 15.9 | 4.0 |
| 42 | 12.5 | 5.1 |
| 43 | 14.7 | 3.6 |
| 44 | 19.5 | 6.8 |
| 45 | 13.3 | 5.3 |
| 46 | 8.2 | 2.7 |
| 47 | 17.2 | 2.5 |
| 48 | 8.4 | 2.3 |
| 49 | 20.8 | 2.7 |
| 50 | 3.9 | 1.7 |
| 51 | 16.3 | 2.9 |
| 52 | 15.5 | 5.8 |
| 53 | 11.8 | 4.7 |
| 54 | 19.9 | 4.9 |
| 55 | 17.3 | 3.7 |
| 56 | 21.1 | 3.3 |
| 57 | 27.2 | 3.7 |
| 58 | 15.8 | 2.6 |
| 59 | 25.7 | 3.9 |
| 60 | 18.1 | 3.8 |
| 61 | 13.6 | 2.2 |
| 62 | 16.2 | 2.6 |
| 63 | 21.6 | 3.6 |
| 64 | 15.8 | 3.3 |
| 65 | 16.1 | 2.6 |
| 66 | 8.7 | 2.7 |
| 67 | 43.6 | 3.6 |

### Preparation A

### 1,2,4-trifluoro-5-(methylsulfonyl)benzene

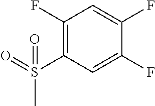

Step A:

To a solution of sodium sulfite (153 g, 1214 mmol) in water (1000 mL) was added a solution of 2,4,5-trifluorobenzene-1-sulfonyl chloride (40 g, 173 mmol) in dioxane (300 mL) dropwise. After the complete addition of sulfonyl chloride, the reaction was basified to about pH 14 by the addition of 1 N NaOH, and the reaction mixture was stirred overnight. The reaction mixture was cooled on an ice bath and acidified using 100 mL concentrated H₂SO₄ to about pH 1. The mixture was extracted with EtOAc and CH₂Cl₂ and the combined organic layers were dried over Na₂SO₄, filtered and concentrated to afford 2,4,5-trifluorobenzenesulfinic acid (34 g, 100%).

Step B:

To a solution of 2,4,5-trifluorobenzenesulfinic acid (34 g, 173 mmol), in DMF (200 mL), was added iodomethane (21.6 mL, 347 mmol), and N-ethyl-N-isopropylpropan-2-amine (60.5 mL, 347 mmol). The reaction mixture was stirred overnight at ambient temperature. The reaction was concentrated and partitioned between water and ethyl acetate and the water layer was extracted with CH₂Cl₂. The combined organic layers were concentrated under vacuum and the product was purified by silica gel chromatography (15-100% EtOAc in hexanes) to afford 1,2,4-trifluoro-5-(methylsulfonyl)benzene (Preparation A; 25.8 g, 123 mmol, 70.8% yield) as yellow solid.

The following compounds were also prepared according to the method of Preparation A.

| Preparation | Structure | Name |
|---|---|---|
| A-1 | 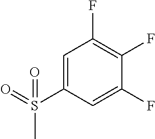 | 1,2,3-trifluoro-5- (methylsulfonyl)benzene |
| A-2 | 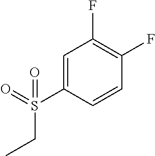 | 4-(ethylsulfonyl)-1,2- difluorobenzene |

### Preparation B

### (R)-tert-butyl 4-(3-(methylsulfonyloxy)-2-oxopyrrolidin-1-yl)piperidine-1-carboxylate

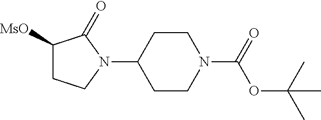 [SMILES: CC(C)(C)OC(=O)N1CCC(N2CC[C@@H](OS(C)(=O)=O)C2=O)CC1]

Step A:

(R)-2-(2,2-Dimethyl-5-oxo-1,3-dioxolan-4-yl)acetic acid (25 g, 144 mmol) was dissolved in CH₂Cl₂ (500 mL) and cooled in an ice bath. Ethanethiol (21.2 mL, 287 mmol) and N,N-dimethylpyridin-4-amine (0.351 g, 2.87 mmol) were added followed by DCC (35.5 g, 172 mmol). This mixture was stirred at 0° C. for 1 hour, and then at ambient temperature for 2 hours. Acetic acid (45 mL) was added and the mixture stirred for 10 minutes. The reaction mixture was then poured into vigorously stirring diethyl ether (400 mL) and filtered. The filtrate was washed with 10% sodium carbonate, water, 0.5 N HCl, water and brine. The organic layer was dried over Na₂SO₄, filtered and concentrated. The residue was purified over silica gel (1-5-10% EtOAc in hexanes) to afford (R)—S-ethyl 2-(2,2-dimethyl-5-oxo-1,3-dioxolan-4-yl)ethanethioate (22.5 g, 103 mmol, 71.8% yield) as an oil that solidified to a white solid.

Step B:

(R)—S-Ethyl 2-(2,2-dimethyl-5-oxo-1,3-dioxolan-4-yl)ethanethioate (22.5 g, 103 mmol) was dissolved in CH₂Cl₂ (500 mL), purged with nitrogen and 10% palladium on carbon (2.19 g, 2.06 mmol) was added. Triethylsilane (24.7 mL, 155 mmol) was dissolved in CH₂Cl₂ (20 mL) and added dropwise through an addition funnel over ˜30 min and the reaction stirred at ambient temperature overnight under nitrogen. The reaction was filtered through celite, concentrated and purified over silica gel (10 to 40% EtOAc in hexanes) to afford (R)-2-(2,2-dimethyl-5-oxo-1,3-dioxolan-4-yl)acetaldehyde (16 g, 101 mmol, 98.1% yield) as an oil.

Step C:

(R)-2-(2,2-Dimethyl-5-oxo-1,3-dioxolan-4-yl)acetaldehyde (16 g, 101 mmol) was dissolved in ClCH₂CH₂Cl (500 mL) and tert-butyl 4-aminopiperidine-1-carboxylate (40.5 g, 202 mmol) and acetic acid (6.94 mL, 121 mmol) were added and the mixture stirred at ambient temperature for 15 minutes. NaBH(OAc)₃ (64.3 g, 304 mmol) was added in 3 portions and the reaction stirred at ambient temperature overnight. The reaction was carefully quenched with saturated aqueous NaHCO₃. The reaction was partitioned between aqueous NaHCO₃ and CH₂Cl₂ and extracted with CH₂Cl₂ (3×200 mL). The organic layer was washed with 10% citric acid, brine, dried over Na₂SO₄, filtered and concentrated under vacuum. The solids were purified over silica gel (5 to 10% methanol in EtOAc) to afford (R)-tert-butyl 4-(3-hydroxy-2-oxopyrrolidin-1-yl)piperidine-1-carboxylate (20.5 g, 72.1 mmol, 71.3% yield) as a white solid.

Step D:

(R)-tert-Butyl 4-(3-hydroxy-2-oxopyrrolidin-1-yl)piperidine-1-carboxylate (20.5 g, 72.1 mmol) was dissolved in THF (500 mL) and triethylamine (20.1 mL, 144 mmol) and methanesulfonyl chloride (6.74 mL, 86.5 mmol) were added to the reaction and stirred at ambient temperature for 1 hour. The reaction was partitioned between saturated aqueous NaHCO₃ and EtOAc, dried over Na₂SO₄, filtered and concentrated under vacuum. The residue was purified over silica gel to afford (R)-tert-butyl 4-(3-(methylsulfonyloxy)-2-oxopyrrolidin-1-yl)piperidine-1-carboxylate (25.5 g, 70.4 mmol, 97.6% yield) as a white solid. ¹H NMR (CDCl₃) 5.2 ppm (t, 1H), 4.3 ppm (m, 2H), 4.1 ppm (m, 1H), 3.4 ppm (m, 1H), 3.3 ppm (m, 1H), 3.3 ppm (s, 3H), 2.8 ppm (m, 2H), 2.6 ppm (m, 1H), 2.3 ppm (m, 1H), 1.7 ppm (m, 2H, 1.6 ppm (m, 2H), 1.5 ppm (s, 9H).

### Preparation C

### S-3-(2-fluoro-4-(methylsulfonyl)phenylamino)-1-(piperidin-4-yl)pyrrolidin-2-one

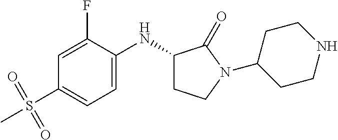 [SMILES: CS(=O)(=O)c1ccc(N[C@H]2CCN(C3CCNCC3)C2=O)c(F)c1]

Step A:

A solution of HBTU (8.1 g, 21 mmol), (S)-2-(tert-butoxycarbonylamino)-4-(methylthio)butanoic acid (5.3 g, 21 mmol) and DIEA (8.2 mL, 47 mmol) in DMF (50 mL) was stirred at ambient temperature for 30 minutes. Benzyl 4-aminopiperidine-1-carboxylate (5.0 g, 21 mmol) was added and the mixture stirred at ambient temperature for 18 hours. The mixture was poured into 1 N NaOH (500 mL) and the organics were extracted into EtOAc (500 mL). The organic layer was washed with 1 N HCl (500 mL) and brine (500 mL), dried over MgSO₄ and concentrated under vacuum to yield (S)-benzyl 4-(2-(tert-butoxycarbonylamino)-4-(methylthio)butanamido)piperidine-1-carboxylate (10 g, 21 mmol, 100%).

Step B:

A solution of (S)-benzyl 4-(2-(tert-butoxycarbonylamino)-4-(methylthio)butanamido)piperidine-1-carboxylate (10 g, 21.5 mmol) in neat MeI (40.2 mL, 640 mmol) was stirred at ambient temperature for 4 hours. The reaction was evaporated to dryness to yield (S)-(4-(1-(benzyloxycarbonyl)piperidin-4-ylamino)-3-(tert-butoxycarbonylamino)-4-oxobutyl)dimethylsulfonium iodide (10 g, 17 mmol, 79%).

Step C:

(S)-(4-(1-(Benzyloxycarbonyl)piperidin-4-ylamino)-3-(tert-butoxycarbonylamino)-4-oxobutyl)dimethylsulfonium iodide (10 g, 17 mmol) was dissolved in dry THF (100 mL) and cooled to 0° C. Lithium bis(trimethylsilyl)amide (21 mL, 21 mmol) was added and the mixture was warmed to ambient temperature and stirred for 2 hours. The mixture was poured into saturated ammonium chloride (100 mL) and extracted into EtOAc (3×100 mL). The organic layer was washed with brine, dried over MgSO₄ and concentrated under vacuum to yield (S)-benzyl 4-(3-(tert-butoxycarbonylamino)-2-oxopyrrolidin-1-yl)piperidine-1-carboxylate (7 g, 17 mmol, 100%).

Step D:

A solution of (S)-benzyl 4-(3-(tert-butoxycarbonylamino)-2-oxopyrrolidin-1-yl)piperidine-1-carboxylate (7 g, 17 mmol) in 50% TFA/CH₂Cl₂ (50 mL) was stirred at ambient temperature for 1 hour and then concentrated under vacuum. The residue was dissolved in EtOAc (200 mL) and washed with saturated sodium carbonate (200 mL) then brine. The organic layer was dried over MgSO₄ and concentrated under vacuum to yield (S)-benzyl 4-(3-amino-2-oxopyrrolidin-1-yl)piperidine-1-carboxylate (3.4 g, 11 mmol, 64%).

Step E:

A solution of (S)-benzyl 4-(3-amino-2-oxopyrrolidin-1-yl)piperidine-1-carboxylate (2.0 g, 6.3 mmol), 1,2-difluoro-4-(methylsulfonyl)benzene (1.2 g, 6.3 mmol), and Na₂CO₃ (3.3 g, 32 mmol) in DMSO (20 mL) was stirred at 120° C. for 48 hours. The reaction mixture was poured into water (200 mL) and extracted with EtOAc (3×100 mL). The organic layer was washed with brine, dried over MgSO₄ and then concentrated under vacuum. The material was purified over silica gel (100% EtOAc) to yield (S)-benzyl 4-(3-(2-fluoro-4-(methylsulfonyl)phenylamino)-2-oxopyrrolidin-1-yl)piperidine-1-carboxylate (1.3 g, 2.7 mmol, 42%).

Step F:

A solution of (S)-benzyl 4-(3-(2-fluoro-4-(methylsulfonyl)phenylamino)-2-oxopyrrolidin-1-yl)piperidine-1-carboxylate (1.3 g, 27 mmol) in ethanol (20 mL) and concentrated HCl (300 μL) was hydrogenated at 40 PSI with 10% Degussa type Pd/C (650 mg) for 18 hours. The mixture was filtered through Celite® and the solids were washed with MeOH (200 mL) and water (200 mL). The methanol in the filtrate was removed under vacuum. The water layer was made basic with 1 N NaOH solution and extracted with dichloromethane. The organic layer was washed with brine, dried over MgSO₄ and concentrated under vacuum to yield (S)-3-(2-fluoro-4-(methylsulfonyl)phenylamino)-1-(piperidin-4-yl)pyrrolidin-2-one (600 mg, 1.7 mmol, 64%). Mass spectrum (apci) m/z=356.1 (M+H).

The following compounds were also prepared according to the method of Preparation C.

| Preparation | Structure | Name | Data |
|---|---|---|---|
| C-1 | 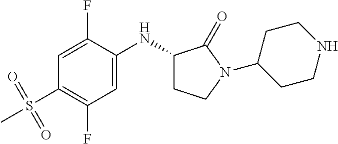 [SMILES: CS(=O)(=O)c1cc(F)c(N[C@H]2CCN(C3CCNCC3)C2=O)cc1F] | (S)-3-(2,5-difluoro-4- (methylsulfonyl)phenyl- amino)-1-(piperidin-4- yl)pyrrolidin-2-one | Mass spectrum (apci) m/z = 374.2 (M + H). |
| C-2 | 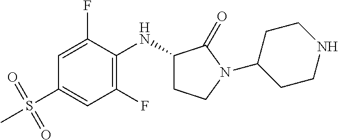 [SMILES: CS(=O)(=O)c1cc(F)c(N[C@H]2CCN(C3CCNCC3)C2=O)c(F)c1] | (S)-3-(2,6-difluoro-4- (methylsulfonyl)phenyl- amino)-1-(piperidin-4- yl)pyrrolidin-2-one | Mass spectrum (apci) m/z = 374.2 (M + H). |
| C-3 | 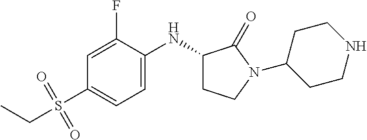 [SMILES: CCS(=O)(=O)c1ccc(N[C@H]2CCN(C3CCNCC3)C2=O)c(F)c1] | (S)-3-(4-(ethylsulfonyl)- 2-fluorophenylamino)- 1-(piperidin-4- yl)pyrrolidin-2-one | Mass spectrum (apci) m/z = 370.2 (M + H). |

### Preparation D

### (S)-3-(2-fluoro-4-(methylsulfonyl)phenoxy)-1-(piperidin-4-yl)pyrrolidin-2-one hydrochloride

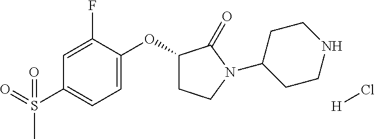

Step A:

(R)-tert-Butyl 4-(3-(methylsulfonyloxy)-2-oxopyrrolidin-1-yl)piperidine-1-carboxylate (Preparation B; 1.7 g, 4.7 mmol) was dissolved in dry DMSO (30 mL) and 4-bromo-2-fluorophenol (1.1 g, 5.6 mmol) and K₂CO₃ (0.78 g, 5.6 mmol) were added and the reaction was heated to 70° C. under nitrogen. The reaction was cooled to ambient temperature after 3 hours and partitioned between water and EtOAc, extracted with EtOAc, washed with brine, dried over Na₂SO₄, filtered and concentrated under vacuum. The residue was purified over silica gel (40% EtOAc in hexanes) to afford (S)-tert-butyl 4-(3-(4-bromo-2-fluorophenoxy)-2-oxopyrrolidin-1-yl)piperidine-1-carboxylate (1.8 g, 3.9 mmol, 84% yield) as a white solid.

Step B:

(S)-tert-Butyl 4-(3-(4-bromo-2-fluorophenoxy)-2-oxopyrrolidin-1-yl)piperidine-1-carboxylate (1.8 g, 3.9 mmol) was dissolved in DMSO (30 mL) and purged with nitrogen. Sodium methanesulfinate (0.60 g, 5.9 mmol) and trans-cyclohexane-1,2-diamine (0.19 mL, 1.6 mmol) were added followed by Cu(I) triflate benzene complex (0.20 g, 0.39 mmol). The reaction was plunged into a 110° C. oil bath under nitrogen and stirred overnight. The reaction was cooled to ambient temperature, partitioned between water and EtOAc, extracted with EtOAc, washed with brine, dried over Na₂SO₄, filtered and concentrated. The residue was purified over silica gel (100% EtOAc) to afford (S)-tert-butyl 4-(3-(2-fluoro-4-(methylsulfonyl)phenoxy)-2-oxopyrrolidin-1-yl)piperidine-1-carboxylate (1.6 g, 3.5 mmol, 89% yield) as a white solid.

Step C:

(S)-tert-Butyl 4-(3-(2-fluoro-4-(methylsulfonyl)phenoxy)-2-oxopyrrolidin-1-yl)piperidine-1-carboxylate (1.6 g, 3.5 mmol) was dissolved in CH₂Cl₂ (20 mL) and 4 N HCl in dioxane (−15 mL) was added and stirred at ambient temperature overnight. The reaction was concentrated to afford (S)-3-(2-fluoro-4-(methylsulfonyl)phenoxy)-1-(piperidin-4-yl)pyrrolidin-2-one hydrochloride (1.5 g, 3.8 mmol, 100% yield) as a white solid. Mass spectrum (apci) m/z=357.2 (M+H).

The following compounds were also prepared according to the method of Preparation D.

| | | | Mass spectrum |
|---|---|---|---|
| Preparation | Structure | Name | (apci) |
| D-1 | 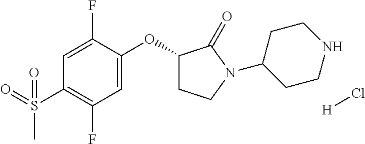 | (S)-3-(2,5-difluoro-4- (methylsulfonyl)- phenoxy)- 1-(piperidin-4- yl)pyrrolidin-2-one hydrochloride | m/z = 375.1 (M + H). |
| D-2 | 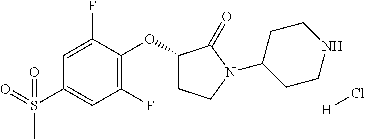 | (S)-3-(2,6-difluoro-4- (methylsulfonyl)- phenoxy)- 1-(piperidin-4- yl)pyrrolidin-2-one hydrochloride | m/z = 375.1 (M + H). |

### Preparation E

### (S)-3-(2-fluoro-4-(methylsulfonyl)phenylamino)-1,4′-bipiperidin-2-one hydrochloride

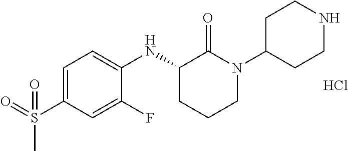 [SMILES: CS(=O)(=O)c1ccc(N[C@H]2CCCN(C3CCNCC3)C2=O)c(F)c1 ; Cl]

Step A:

(S)-5-Amino-2-(benzyloxycarbonylamino)pentanoic acid (5.0 g, 19 mmol) was dissolved in THF (100 mL) and water (20 mL). tert-Butyl 4-oxopiperidine-1-carboxylate (3.7 g, 19 mmol) was added and the reaction mixture was stirred for 1 hour. The reaction was cooled to 0° C., then 1.0 M NaCNBH₃ in THF (19 mL, 19 mmol) was added and the mixture was allowed to stir at ambient temperature overnight. The solvent was removed under vacuum to leave crude (S)-2-(benzyloxycarbonylamino)-5-(1-(tert-butoxycarbonyl)piperidin-4-ylamino)pentanoic acid (8.4 g, 19 mmol, 100% yield) which was taken forward without further purification.

Step B:

Crude (S)-2-(benzyloxycarbonylamino)-5-(1-(tert-butoxycarbonyl)piperidin-4-ylamino)pentanoic acid (8.4 g, 18.7 mmol) was dissolved in DMF (100 mL) and cooled to 0° C. EDCI (3.58 g, 18.7 mmol) and N-ethyl-N-isopropylpropan-2-amine (3.25 mL, 18.7 mmol) were added and the reaction was allowed to warm to ambient temperature overnight. The reaction was diluted with EtOAc and washed with 1 N HCl, saturated aqueous NaHCO₃ and brine. The organic layer was dried over Na₂SO₄, filtered and concentrated under vacuum. The residue was purified over silica gel (50 to 80% EtOAc in hexanes) to afford (S)-tert-butyl 3-(benzyloxycarbonylamino)-2-oxo-1,4′-bipiperidine-1′-carboxylate (5.2 g, 12.1 mmol, 64.5% yield).

Step C:

(S)-tert-Butyl 3-(benzyloxycarbonylamino)-2-oxo-1,4′-bipiperidine-1′-carboxylate (5.2 g, 12 mmol) was dissolved in methanol (100 mL) and 10% Pd/C (500 mg) was added and stirred under balloon pressure of hydrogen for 3 hours. The reaction was filtered through celite and concentrated to afford (S)-tert-butyl 3-amino-2-oxo-1,4′-bipiperidine-1′-carboxylate (4.2 g, 14 mmol, 117% yield) as a pale yellow oil. The crude material was used directly in the next step without further purification.

Step D:

(S)-tert-Butyl 3-amino-2-oxo-1,4′-bipiperidine-1′-carboxylate (1.0 g, 3.36 mmol) was dissolved in DMSO (20 mL) and 1,2-difluoro-4-(methylsulfonyl)benzene (0.775 g, 4.04 mmol) and Na₂CO₃ (0.535 g, 5.04 mmol) were added and the reaction heated to 120° C. under nitrogen overnight. The reaction was cooled to ambient temperature, water was added and the reaction mixture was extracted with EtOAc, dried over Na₂SO₄, filtered and concentrated under vacuum. The residue was purified over silica gel (80% EtOAc in hexanes) to afford (S)-tert-butyl 3-(2-fluoro-4-(methylsulfonyl)phenylamino)-2-oxo-1,4′-bipiperidine-1′-carboxylate (680 mg, 1.45 mmol, 43.1% yield) as a white solid. Mass spectrum (apci) m/z=370.2 (M+H-Boc).

Step E:

(S)-tert-Butyl 3-(2-fluoro-4-(methylsulfonyl)phenylamino)-2-oxo-1,4′-bipiperidine-1′-carboxylate (7.9 g, 17 mmol) was dissolved in CH₂Cl₂ (100 mL) and 4 N HCl in dioxane (30 mL) was added and stirred at ambient temperature overnight. The reaction was concentrated to afford (S)-3-(2-fluoro-4-(methylsulfonyl)phenylamino)-1,4′-bipiperidin-2-one hydrochloride (6.2 g, 17 mmol, 100% yield) as a white solid. Mass spectrum (apci) m/z=370.2 (M+H).

The following compounds were also prepared according to the method of Preparation E.

| | | | Mass spectrum |
|---|---|---|---|
| Preparation | Structure | Name | (apci) |
| E-1 | 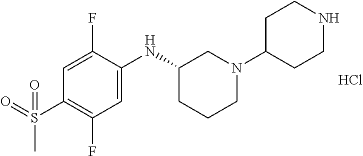 | (S)-3-(2,5-difluoro-4- (methylsulfonyl)- phenylamino)- 1,4′-bipiperidin-2-one hydrochloride | m/z = 388.1 (M + H). |
| E-2 | 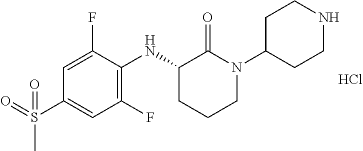 [SMILES: CS(=O)(=O)c1cc(F)c(N[C@H]2CCCN(C3CCNCC3)C2=O)c(F)c1 ; Cl] | (S)-3-(2,6-difluoro-4- (methylsulfonyl)- phenylamino)- 1,4′-bipiperidin-2-one hydrochloride | m/z = 388.1 (M + H). |

### Preparation F

### (S)-3-(2-fluoro-4-(methylsulfonyl)phenylamino)-1,4′-bipiperidin-2-one

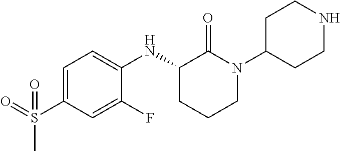 [SMILES: CS(=O)(=O)c1ccc(N[C@H]2CCCN(C3CCNCC3)C2=O)c(F)c1]

(S)-tert-Butyl 3-(2-fluoro-4-(methylsulfonyl)phenylamino)-2-oxo-1,4′-bipiperidine-1′-carboxylate (Preparation E, Steps A-D; 570 mg, 1.2 mmol) was dissolved in dichloromethane (10 mL) and 2,2,2-trifluoroacetic acid (2.8 g, 24.3 mmol) was added and the reaction stirred at ambient temperature for 30 minutes. The reaction was concentrated under vacuum and partitioned between saturated sodium bicarbonate solution and dichloromethane. The organic layer was separated and the aqueous layer was further extracted with 10% MeOH in dichloromethane (3 times). The organic layers were combined, dried with MgSO₄, and concentrated to afford (S)-3-(2-fluoro-4-(methylsulfonyl)phenylamino)-1,4′-bipiperidin-2-one (260 mg, 58%); Mass spectrum (apci) m/z=370.0 (M+H).

The following compounds were also prepared according to Preparation F.

| | | | Mass spectrum |
|---|---|---|---|
| Preparation | Structure | Name | (apci) |
| F-1 | 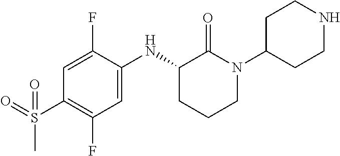 [SMILES: CS(=O)(=O)c1cc(F)c(N[C@H]2CCCN(C3CCNCC3)C2=O)cc1F] | (S)-3-(2,5-difluoro-4- (methylsulfonyl)phenyl- amino)-1,4′- bipiperidin-2-one | m/z = 388.2 (M + H) |
| F-2 | 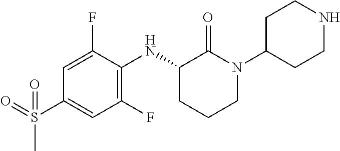 [SMILES: CS(=O)(=O)c1cc(F)c(N[C@H]2CCCN(C3CCNCC3)C2=O)c(F)c1] | (S)-3-(2,6-difluoro-4- (methylsulfonyl)phenyl- amino)-1,4'- bipiperidin-2-one | m/z = 388.2 (M + H) |
| F-3 | 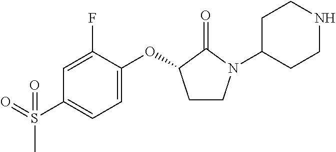 [SMILES: CS(=O)(=O)c1ccc(O[C@H]2CCN(C3CCNCC3)C2=O)c(F)c1] | (S)-3-(2-fluoro-4- (methylsulfonyl)phenoxy)- 1-(piperidin-4- yl)pyrrolidin-2-one | m/z = 357.2 (M + H) |
| F-4 | 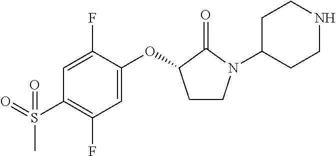 [SMILES: CS(=O)(=O)c1cc(F)c(O[C@H]2CCN(C3CCNCC3)C2=O)cc1F] | (S)-3-(2,5-difluoro-4- (methylsulfonyl)phenoxy)- 1-(piperidin-4- yl)pyrrolidin-2-one | m/z = 375.1 (M + H) |

### Preparation G

### (S)-3-((6-(methylsulfonyl)pyridin-3-yl)oxy)-1-(piperidin-4-yl)pyrrolidin-2-one

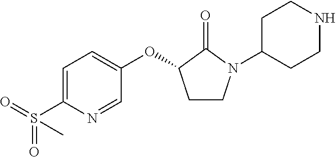 [SMILES: CS(=O)(=O)c1ccc(O[C@H]2CCN(C3CCNCC3)C2=O)cn1]

Step A:

To a suspension of (R)-3-hydroxydihydrofuran-2(3H)-one (500 mg, 4.9 mmol) in toluene (15 mL) was added triphenylphosphine (1.54 g, 5.88 mmol) and 6-bromopyridin-3-ol (1.02 g, 5.88 mmol). The solution was cooled to 0° C. and degassed with nitrogen for 10 minutes. Di-tert-butyl diazene-1,2-dicarboxylate was dissolved in toluene (5 mL) and added over a 5 minute period. The reaction was allowed to stir for 12 hours with warming to ambient temperature. The reaction was concentrated in vacuo and the resulting material was purified by silica gel chromatography, eluting 1:1 hexanes/EtOAc, to yield (S)-3-(6-bromopyridin-3-yloxy)dihydrofuran-2(3H)-one (1.0 g, 79%) as an off-white solid.

Step B:

To a solution of tert-butyl 4-aminopiperidine-1-carboxylate (0.93 g, 4.65 mmol) in DCM (15 mL) was added dropwise 2M trimethylaluminum (2.8 mL, 5.7 mmol) in toluene. The resulting mixture was stirred for 15 minutes. (S)-3-(6-Bromopyridin-3-yloxy)dihydrofuran-2(3H)-one (1.0 g, 3.87 mmol) in DCM (10 mL) was added slowly over 5 minutes and the reaction stirred at ambient temperature for 2 hours. The reaction was slowly quenched by the addition of 5% tartaric acid (5 mL), saturated NaHCO₃ (5 mL) and DCM (10 mL). The organic layer was separated, dried over MgSO₄, filtered and concentrated in vacuo to provide (S)-tert-butyl 4-(2-(6-bromopyridin-3-yloxy)-4-hydroxybutanamido)piperidine-1-carboxylate. The crude material was taken on to the next step without a further purification.

Step C:

A solution of (S)-tert-butyl 4-(2-(6-bromopyridin-3-yloxy)-4-hydroxybutanamido)piperidine-1-carboxylate (1.30 g, 2.84 mmol) and tributylphosphine (689 mg, 3.40 mmol) in toluene (15 mL) was degassed with nitrogen for 10 minutes and then cooled to 0° C. Di-tert-butyl diazene-1,2-dicarboxylate (784 mg, 3.4 mmol) dissolved in toluene (5 mL) and the solution was added to the reaction mixture over a 5 minute period. The reaction was allowed to warm to ambient temperature over 12 hours. The reaction was concentrated in vacuo and purified by silica gel chromatography eluting with 1:1 hexanes/EtOAc to yield (S)-tert-butyl-4-(3-(6-bromopyridin-3-yloxy)-2-oxopyrrolidin-1-yl)piperidine-1-carboxylate (934 mg, 75%) as an off-white solid.

Step D:

A solution of (S)-tert-butyl-4-(3-(6-bromopyridin-3-yloxy)-2-oxopyrrolidin-1-yl)piperidine-1-carboxylate (315 mg, 0.714 mmol) in degassed DMSO (5 mL) was added sodium methane sulfinate (117 mg, 1.15 mmol), trans-cyclohexane-1,2-diamine (33 mg, 0.286 mmol) and Cu(I) triflate benzene complex (54 mg, 0.107 mmol). The reaction was heated to 110° C. for 12 hours at which point the reaction was cooled to ambient temperature and partitioned between water (5 mL) and EtOAc (10 mL). The organic layer was separated and the aqueous layer extracted with EtOAc (2×5 mL). The combined organic layers were dried over MgSO₄, filtered and concentrated in vacuo. The resulting material was purified by silica gel chromatography eluting with 1:1 hexanes/EtOAc to yield (S)-tert-butyl-4-(3-(6-(methylsulfonyl)pyridine-3-yloxy)-2-oxopyrrolidin-1-yl)piperidine-1-carboxylate (315 mg, 94%) as a white solid.

Step E:

Trifluoroacetic acid (1 mL) was added to a solution of (S)-tert-butyl 4-(3-(6-(methylsulfonyl)pyridin-3-yloxy)-2-oxopyrrolidin-1-yl)piperidine-1-carboxylate (0.092 g, 0.21 mmol) in CH₂Cl₂ (2 mL). The solution was stirred at ambient temperature overnight, then concentrated in vacuo. The residue was diluted with MeOH, concentrated, then stirred in CHCl₃ (20 mL) and 1 M NaOH (20 mL) for 5 minutes. The layers were separated and the aqueous phase was extracted twice with CHCl₃ (10 mL each). The combined organics were dried (MgSO₄) and concentrated to afford (S)-3-(6-(methylsulfonyl)pyridin-3-yloxy)-1-(piperidin-4-yl)pyrrolidin-2-one (64 mg, 90%) of a white foam.

### Preparation H

### (S)-1-(1-(3,5-dichloropyrazin-2-yl)piperidin-4-yl)-3-(6-(methylsulfonyl)pyridin-3-ylamino)pyrrolidin-2-one

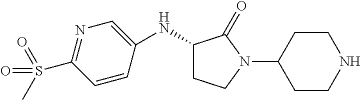 [SMILES: CS(=O)(=O)c1ccc(N[C@H]2CCN(C3CCNCC3)C2=O)cn1]

Step A:

A solution of HBTU (8.1 g, 21 mmol), (S)-2-(tert-butoxycarbonylamino)-4-(methylthio)butanoic acid (5.3 g, 21 mmol) and DIEA (8.2 mL, 47 mmol) in DMF (50 mL) was stirred at ambient temperature for 30 minutes. Benzyl 4-aminopiperidine-1-carboxylate (5.0 g, 21 mmol) was added and the mixture was stirred at ambient temperature for 18 hours. The mixture was poured into 1N NaOH (500 mL) and extracted into EtOAc (500 mL). The combined organic layers were washed with 1N HCl (500 mL) and brine (500 mL)., dried over MgSO₄, filtered and concentrated in vacuo to yield (S)-benzyl 4-(2-(tert-butoxycarbonylamino)-4-(methylthio)butanamido)piperidine-1-carboxylate (10 g, 21 mmol, 100%).

Step B:

A solution of (S)-benzyl 4-(2-(tert-butoxycarbonylamino)-4-(methylthio)butanamido)piperidine-1-carboxylate (10 g, 21.5 mmol) in neat MeI (40.2 mL, 640 mmol) was stirred at ambient temperature for 4 hours. The reaction was evaporated to dryness to yield (S)-benzyl 4-(3-(2-fluoro-4-(methylsulfonyl)phenylamino)-2-oxopyrrolidin-1-yl)piperidine-1-carboxylate methiodide salt (10 g, 17 mmol, 79%).

Step C:

The (S)-Benzyl 4-(3-(2-fluoro-4-(methylsulfonyl)phenylamino)-2-oxopyrrolidin-1-yl)piperidine-1-carboxylate methiodide salt (10 g, 17 mmol) was dissolved in dry THF (100 mL) and cooled to 0° C. Lithium bis(trimethylsilyl)amide (21 mL, 21 mmol) was added and the mixture was warmed to ambient temperature and stirred for 2 hours. The mixture was poured into saturated ammonium chloride (100 mL) and extracted into EtOAc (3×100 mL). The combined organic layers were washed with brine, dried over MgSO₄, filtered and concentrated in vacuo to yield (S)-benzyl 4-(3-(tert-butoxycarbonylamino)-2-oxopyrrolidin-1-yl)piperidine-1-carboxylate (7 g, 17 mmol, 100%).

Step D:

A solution of (S)-benzyl 4-(3-(tert-butoxycarbonylamino)-2-oxopyrrolidin-1-yl)piperidine-1-carboxylate (7 g, 17 mmol) in 50% TFA/CH₂Cl₂ (50 mL) was stirred at ambient temperature for 1 hour. The mixture was concentrated in vacuo. The residue was dissolved in EtOAc (200 mL) and washed with saturated sodium carbonate (200 mL) and brine. The combined organic layers were dried over MgSO₄, filtered and concentrated in vacuo to yield (S)-benzyl 4-(3-amino-2-oxopyrrolidin-1-yl)piperidine-1-carboxylate (3.4 g, 11 mmol, 64%).

Step E:

(S)-Benzyl 4-(3-amino-2-oxopyrrolidin-1-yl)piperidine-1-carboxylate (Preparation E, Step D; 0.26 g, 0.82 mmol), 5-bromo-2-(methylsulfonyl)pyridine (0.26 g, 1.10 mmol), Pd₂ dba₃ (0.038 g, 0.041 mmol), Binap-rac (0.051 g, 0.082 mmol), and cesium carbonate (0.43 g, 1.30 mmol) were added to an argon-filled sealable flask. DMA (7.5 mL) was added and the system was purged with bubbling argon for 5 minutes. The system was sealed and stirred at 100° C. overnight. The mixture was cooled to ambient temperature and diluted with THF (60 mL) and stirred for 30 minutes. The mixture was filtered through GF/F paper and concentrated under vacuum. The residue was purified by silica chromatography (EtOAc) to afford (S)-benzyl 4-(3-(6-(methylsulfonyl)pyridin-3-ylamino)-2-oxopyrrolidin-1-yl)piperidine-1-carboxylate (0.23 g, 0.49 mmol, 51%).

Step F:

(S)-Benzyl 4-(3-(6-(methylsulfonyl)pyridin-3-ylamino)-2-oxopyrrolidin-1-yl)piperidine-1-carboxylate (0.23 g, 0.49 mmol) was dissolved in MeOH (5 mL) and cooled to 0° C. The material was purged with N₂ by three vacuum pump/N₂ balloon cycles. Palladium on carbon (10 wt. % dry basis, wet, Degussa type, 0.053 g, 0.49 mmol) was added and the system was purged with 1 atm H₂ by three vacuum pump/H₂ balloon cycles. The reaction continued to stir under H₂ until the starting material was consumed. The mixture was filtered through GF/F paper and concentrated to give (S)-3-(6-(methylsulfonyl)pyridin-3-ylamino)-1-(piperidin-4-yl)pyrrolidin-2-one (0.17 g, 0.51 mmol, 100% yield). Optical purity of the product was not assessed.

### Example 1

### (S)-3-(2,5-difluoro-4-(methylsulfonyl)phenoxy)-1-(1-(3-isopropyl-1,2,4-oxadiazol-5-yl)piperidin-4-yl)pyrrolidin-2-one

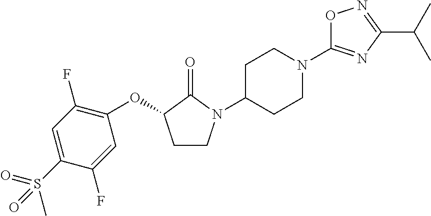 [SMILES: CC(C)c1noc(N2CCC(N3CC[C@H](Oc4cc(F)c(S(C)(=O)=O)cc4F)C3=O)CC2)n1]

Step A:

To a solution of cyanic bromide (0.24 g, 2.3 mmol) in acetonitrile (40 mL) was added potassium carbonate (0.37 g, 2.7 mmol) and (S)-3-(2,5-difluoro-4-(methylsulfonyl)phenoxy)-1-(piperidin-4-yl)pyrrolidin-2-one (Preparation F-4; 0.50 g, 1.34 mmol) and the reaction stirred for 1.5 hours at ambient temperature. The reaction was then quenched with 1 N NaOH. The material was extracted with EtOAc and the separated organic layer was washed with 1 N NaOH, brine, and dried over MgSO₄. The organic layer was concentrated under vacuum to give (S)-4-(3-(2,5-difluoro-4-(methylsulfonyl)phenoxy)-2-oxopyrrolidin-1-yl)piperidine-1-carbonitrile (0.48 g, 1.2 mmol, 90% yield) as a white solid.

Step B:

To a solution of (S)-4-(3-(2,5-difluoro-4-(methylsulfonyl)phenoxy)-2-oxopyrrolidin-1-yl)piperidine-1-carbonitrile (0.48 g, 1.20 mmol) in dioxane (10 mL) was added N-hydroxyisobutyrimidamide (0.18 g, 1.8 mmol) and ZnCl₂ (0.25 g, 1.81 mmol). The reaction stirred overnight at 100° C. The solution was cooled and 1 N NaOH was added and the solution was extracted with EtOAc, dried over MgSO₄, and concentrated under vacuum. The residue was purified by flash silica gel chromatography to provide (S)-3-(2,5-difluoro-4-(methylsulfonyl)phenoxy)-1-(1-(3-isopropyl-1,2,4-oxadiazol-5-yl)piperidin-4-yl)pyrrolidin-2-one (0.11 g, 0.23 mmol, 19% yield). Mass spectrum (apci) m/z=485.2 (M+H). ¹H NMR (400 MHz, CDCl₃) δ 1.30 (d, 6H), 1.70-1.90 (m, 4H), 2.3-2.40 (m, 1H), 2.55 (2.63 (m, 1H), 2.90 (sept, 1H), 3.10-3.20 (m, 2H), 3.21 (s, 3H), 3.33-3.41 (m, 1H), 3.51-3.59 (m, 1H), 4.18-4.28 (m, 1H), 4.25-4.33 (m, 2H), 4.99 (t, 1H), 7.37 (dd, 1H), 7.67 (dd, 1H).

### Example 2

### (S)-3-(2-fluoro-4-(methylsulfonyl)phenoxy)-1-(1-(3-isopropyl-1,2,4-oxadiazol-5-yl)piperidin-4-yl)pyrrolidin-2-one

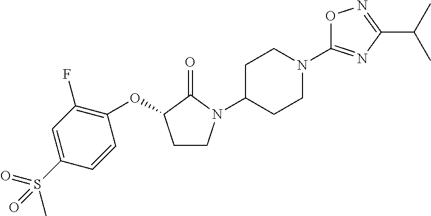 [SMILES: CC(C)c1noc(N2CCC(N3CC[C@H](Oc4ccc(S(C)(=O)=O)cc4F)C3=O)CC2)n1]

Prepared according to the method of Example 1, using (S)-3-(2-fluoro-4-(methylsulfonyl)phenoxy)-1-(piperidin-4-yl)pyrrolidin-2-one (Preparation F-3) in Step A. Mass spectrum (apci) m/z=467.2 (M+H).

### Example 3

### (S)-1-(1-(3-tert-butyl-1,2,4-oxadiazol-5-yl)piperidin-4-yl)-3-(2,5-difluoro-4-(methylsulfonyl)phenoxy)pyrrolidin-2-one

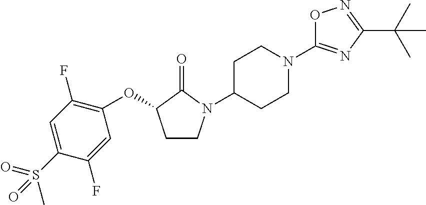 [SMILES: CC(C)(C)c1noc(N2CCC(N3CC[C@H](Oc4cc(F)c(S(C)(=O)=O)cc4F)C3=O)CC2)n1]

Prepared according to the method of Example 1, using (S)-3-(2,5-difluoro-4-(methylsulfonyl)phenoxy)-1-(piperidin-4-yl)pyrrolidin-2-one (Preparation F-4) in Step A and using N-hydroxypivalimidamide in Step B. Mass spectrum (apci) m/z=499.2 (M+H).

### Example 4

### (R)-3-(2,5-difluoro-4-(methylsulfonyl)phenoxy)-1-(1-(3-isopropyl-1,2,4-oxadiazol-5-yl)piperidin-4-yl)pyrrolidin-2-one

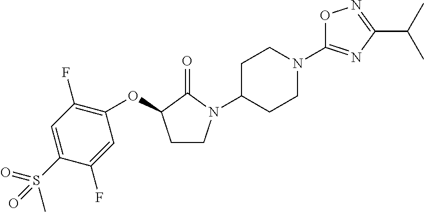 [SMILES: CC(C)c1noc(N2CCC(N3CC[C@@H](Oc4cc(F)c(S(C)(=O)=O)cc4F)C3=O)CC2)n1]

Step A:

tert-Butyl 4-aminopiperidine-1-carboxylate (10.0 g, 50.0 mmol) was suspended in dichloromethane. Triethylamine (7.6 g, 75 mmol) was added and reaction mixture was cooled to 0° C. 2,4-Dibromobutanoyl chloride (13.2 g 50 mmol) was added over a 1 minute period. After 4 hours, the reaction was poured into a saturated NaHCO₃ solution and extracted with dichloromethane. The organic layer was dried, filtered and concentrated to afford tert-butyl 4-(2,4-dibromobutanamido)piperidine-1-carboxylate (22 g, 100%) which was used in the next step without any further purification.

Step B:

tert-Butyl 4-(2,4-dibromobutanamido)piperidine-1-carboxylate (22 g, 51.6 mmol) was dissolved in DMF (100 mL) and cooled to 0° C. Sodium hydride (2.37 g, 60% dispersion in mineral oil) was added and the reaction stirred overnight at ambient temperature. The reaction was partitioned between water and EtOAc. The organic layer was dried, filtered and concentrated. The residue was purified by flash chromatography (eluting with 1:1 to 2:3 hexanes:EtOAc) to give tert-butyl 4-(3-bromo-2-oxopyrrolidin-1-yl)piperidine-1-carboxylate (11.2 g, 63%).

Step C:

To a solution of potassium carbonate (4.78 g, 34.6 mmol) in acetone was added 4-bromo-2,5-difluorophenol (4.87 g, 23.3 mmol) and the reaction stirred for 10 minutes. tert-Butyl 4-(3-bromo-2-oxopyrrolidin-1-yl)piperidine-1-carboxylate (6.0 g, 17.3 mmol) was added and the reaction stirred overnight at ambient temperature. The reaction was concentrated and the residue partitioned between EtOAc and 1N NaOH solution. The organic layer was washed with water and brine, dried over MgSO₄, filtered and concentrated. The crude material was purified by flash chromatography (eluting with 20% EtOAc/dichloromethane) to give tert-butyl 4-(3-(4-bromo-2,5-difluorophenoxy)-2-oxopyrrolidin-1-yl)piperidine-1-carboxylate (5.7 g, 69%).

Step D:

A solution of tert-butyl 4-(3-(4-bromo-2,5-difluorophenoxy)-2-oxopyrrolidin-1-yl)piperidine-1-carboxylate (5.6 g, 11.8 mmol) in DMSO (30 mL) was purged with nitrogen gas for 30 minutes. (1R,2R)-cyclohexane-1,2-diamine (0.54 g, 4.71 mmol), sodium methanesulfinate (1.68 g, 16.5 mmol) and Cu(I) triflate-benzene complex (0.59 g, 1.2 mmol) were added and the reaction was stirred for 2 days at 100° C. The reaction was poured into water and extracted with EtOAc. The combined organic extracts were washed with water and brine, dried over MgSO₄, filtered and concentrated. The crude material was purified by flash chromatography (eluting with 15% EtOAc/dichloromethane to 100% EtOAc) to provide tert-butyl 4-(3-(2,5-difluoro-4-(methylsulfonyl)phenoxy)-2-oxopyrrolidin-1-yl)piperidine-1-carboxylate (2.35, 42%).

Step E:

To a solution of tert-butyl 4-(3-(2,5-difluoro-4-(methylsulfonyl)phenoxy)-2-oxopyrrolidin-1-yl)piperidine-1-carboxylate (2.3 g, 4.8 mmol) in dichloromethane was added 2,2,2-trifluoroacetic acid (11 g, 97 mmol) and the reaction stirred for 2 hours at ambient temperature. The reaction was concentrated and the material was partitioned between EtOAc and 1N NaOH solution. The layers were separated and the organic layer was dried over MgSO₄, filtered and concentrated to give 3-(2,5-difluoro-4-(methylsulfonyl)phenoxy)-1-(piperidin-4-yl)pyrrolidin-2-one (1.5 g, 83%). Mass spectrum (apci) m/z=375.1 (M+H).

Step F:

To a solution of cyanic bromide (0.096 g, 0.91 mmol) in acetonitrile was added potassium carbonate (0.15 g, 1.1 mmol) and 3-(2,5-difluoro-4-(methylsulfonyl)phenoxy)-1-(piperidin-4-yl)pyrrolidin-2-one (0.20 g, 0.53 mmol) and the reaction stirred for 1.5 hours at ambient temperature. The reaction was poured into a water/EtOAc mixture and the water layer was made basic with 1N NaOH solution. The organic layer was separated, washed with brine, dried over MgSO₄, filtered and concentrated to give 4-(3-(2,5-difluoro-4-(methylsulfonyl)phenoxy)-2-oxopyrrolidin-1-yl)piperidine-1-carbonitrile (0.19 g, 89%). Mass spectrum (apci) m/z=400.1 (M+H).

Step G:

To a solution of 4-(3-(2,5-difluoro-4-(methylsulfonyl)phenoxy)-2-oxopyrrolidin-1-yl)piperidine-1-carbonitrile (0.19 g, 0.48 mmol) in EtOAc was added N-hydroxyisobutyrimidamide (0.058 g, 0.57 mmol) and zinc(II) bromide (0.13 g, 0.57 mmol) and the reaction was stirred overnight at ambient temperature. The reaction was diluted with ether and the solid filtered and washed with ether. The solids were taken up in a 2:1 mixture of ethanol/concentrated HCl (15 mL total) and stirred at 90° C. for 2 hours. The reaction was then cooled and charged with 1N NaOH solution and extracted with EtOAc. The organic layer was washed with brine, dried over MgSO₄, filtered and concentrated. The crude material was purified by flash chromatography (eluting with EtOAc) to give 3-(2,5-difluoro-4-(methylsulfonyl)phenoxy)-1-(1-(3-isopropyl-1,2,4-oxadiazol-5-yl)piperidin-4-yl)pyrrolidin-2-one (0.031 g, 13% yield). This material was separated into its two enantiomers by chiral chromatography (OJ-H column, 4.6×150 mm, eluting with (1:1) hexanes/EtOH) to give (R)-3-(2,5-difluoro-4-(methylsulfonyl)phenoxy)-1-(1-(3-isopropyl-1,2,4-oxadiazol-5-yl)piperidin-4-yl)pyrrolidin-2-one (7.6 mg) as a white solid. This enantiomer was found to have a different chiral retention time than that shown for Example 1 but the same mass spectrum and ¹HNMR spectra. Mass spectrum (apci) m/z=485.2 (M+H). ¹H NMR (400 MHz, CDCl₃) δ 1.30 (d, 6H), 1.70-1.90 (m, 4H), 2.3-2.40 (m, 1H), 2.55 (2.63 (m, 1H), 2.90 (sept, 1H), 3.10-3.20 (m, 2H), 3.21 (s, 3H), 3.33-3.41 (m, 1H), 3.51-3.59 (m, 1H), 4.18-4.28 (m, 1H), 4.25-4.33 (m, 2H), 4.99 (t, 1H), 7.37 (dd, 1H), 7.67 (dd, 1H).

### Example 5

### (S)-1-(1-(3-tert-butyl-1,2,4-oxadiazol-5-yl)piperidin-4-yl)-3-(2-fluoro-4-(methylsulfonyl)phenoxy)pyrrolidin-2-one

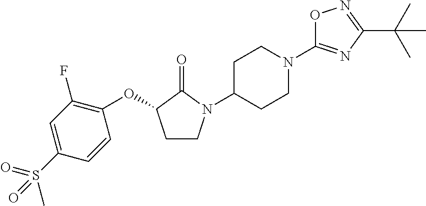 [SMILES: CC(C)(C)c1noc(N2CCC(N3CC[C@H](Oc4ccc(S(C)(=O)=O)cc4F)C3=O)CC2)n1]

Prepared according to the method of Example 1, using (S)-3-(2-fluoro-4-(methylsulfonyl)phenoxy)-1-(piperidin-4-yl)pyrrolidin-2-one (Preparation F-3) in Step A and using N-hydroxypivalimidamide in Step B. Mass spectrum (apci) m/z=481.2 (M+H).

### Example 6

### (S)-1-(1-(3-ethyl-1,2,4-oxadiazol-5-yl)piperidin-4-yl)-3-(2-fluoro-4-(methylsulfonyl)phenoxy)pyrrolidin-2-one

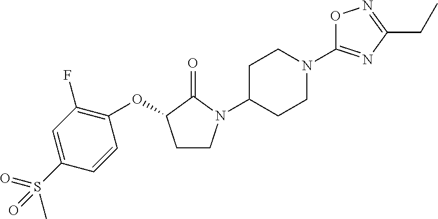 [SMILES: CCc1noc(N2CCC(N3CC[C@H](Oc4ccc(S(C)(=O)=O)cc4F)C3=O)CC2)n1]

Prepared according to the method of Example 1, using (S)-3-(2-fluoro-4-(methylsulfonyl)phenoxy)-1-(piperidin-4-yl)pyrrolidin-2-one (Preparation F-3) in Step A and using N-hydroxypropionimidamide in Step B. Mass spectrum (apci) m/z=453.2 (M+H).

### Example 7

### (S)-3-(2-fluoro-4-(methylsulfonyl)phenylamino)-1′-(3-isopropyl-1,2,4-oxadiazol-5-yl)-1,4′-bipiperidin-2-one

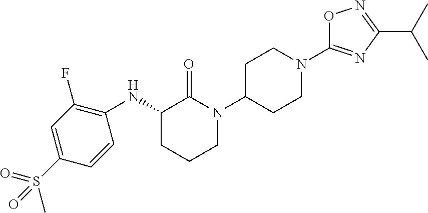 [SMILES: CC(C)c1noc(N2CCC(N3CCC[C@H](Nc4ccc(S(C)(=O)=O)cc4F)C3=O)CC2)n1]

Prepared according to the method of Example 1, using (S)-3-(2-fluoro-4-(methylsulfonyl)phenylamino)-1,4′-bipiperidin-2-one hydrochloride (Preparation E) and 2 equivalents of base in Step A. Mass spectrum (apci) m/z=480.2 (M+H).

### Example 8

### (S)-3-(2-fluoro-4-(methylsulfonyl)phenylamino)-1-(1-(3-isopropyl-1,2,4-oxadiazol-5-yl)piperidin-4-yl)pyrrolidin-2-one

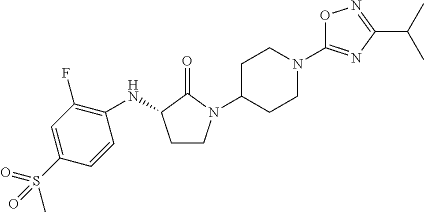 [SMILES: CC(C)c1noc(N2CCC(N3CC[C@H](Nc4ccc(S(C)(=O)=O)cc4F)C3=O)CC2)n1]

Prepared according to the method of Example 1, using (S)-3-(2-fluoro-4-(methylsulfonyl)phenylamino)-1-(piperidin-4-yl)pyrrolidin-2-one (Preparation C) in Step A. Mass spectrum (apci) m/z=466.2 (M+H).

### Example 9

### (S)-3-(2,5-difluoro-4-(methylsulfonyl)phenylamino)-1′-(3-trifluoromethyl-1,2,4-oxadiazol-5-yl)-1,4′-bipiperidin-2-one

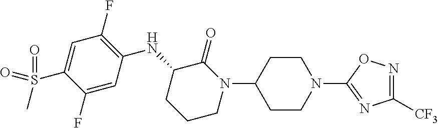 [SMILES: CS(=O)(=O)c1cc(F)c(N[C@H]2CCCN(C3CCN(c4nc(C(F)(F)F)no4)CC3)C2=O)cc1F]

Prepared according to the method of Example 1, using (S)-3-(2,5-difluoro-4-(methylsulfonyl)phenylamino)-1,4′-bipiperidin-2-one hydrochloride (Preparation E-1) in Step A and using 2,2,2-trifluoro-N-hydroxyacetimidamide in Step B. Mass spectrum (apci) m/z=522 (M−H).

### Example 10

### (S)-3-(2,6-difluoro-4-(methylsulfonyl)phenylamino)-1′-(3-trifluoromethyl-1,2,4-oxadiazol-5-yl)-1,4′-bipiperidin-2-one

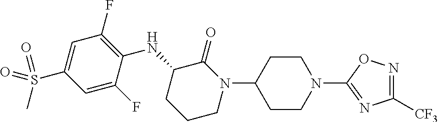 [SMILES: CS(=O)(=O)c1cc(F)c(N[C@H]2CCCN(C3CCN(c4nc(C(F)(F)F)no4)CC3)C2=O)c(F)c1]

Prepared according to the method of Example 1, using (S)-3-(2,6-difluoro-4-(methylsulfonyl)phenylamino)-1,4′-bipiperidin-2-one (Preparation F-2) in Step A and using 2,2,2-trifluoro-N-hydroxyacetimidamide in Step B. Mass spectrum (apci) m/z=522 (M−H).

### Example 11

### (S)-1-(1-(3-trifluoromethyl-1,2,4-oxadiazol-5-yl)piperidin-4-yl)-3-(2,5-difluoro-4-(methylsulfonyl)phenoxy)pyrrolidin-2-one

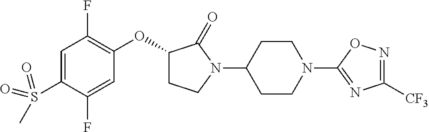 [SMILES: CS(=O)(=O)c1cc(F)c(O[C@H]2CCN(C3CCN(c4nc(C(F)(F)F)no4)CC3)C2=O)cc1F]

Prepared according to the method of Example 1, using (S)-3-(2,5-difluoro-4-(methylsulfonyl)phenoxy)-1-(piperidin-4-yl)pyrrolidin-2-one (Preparation F-4) in Step A and using 2,2,2-trifluoro-N-hydroxyacetimidamide in Step B. ¹H NMR (400 MHz, CDCl₃) δ 1.72-1.93 (m, 4H), 2.30-2.40 (m, 1H), 2.58-2.68 (m, 1H), 3.19 (s, 3H), 3.20-3.30 (m, 2H), 3.35-3.42 (m, 1H), 3.50-3.59 (m, 1H), 4.20-4.29 (m, 1H), 4.32-4.39 (m, 2H), 4.99 (t, 1H), 7.35 (dd, 1H), 7.63 (dd, 1H).

### Example 12

### (S)-3-(2,6-difluoro-4-(methylsulfonyl)phenylamino)-1′-(5-isopropyl-1,2,4-oxadiazol-3-yl)-1,4′-bipiperidin-2-one

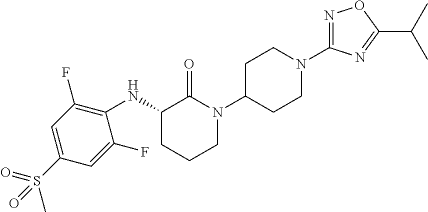 [SMILES: CC(C)c1nc(N2CCC(N3CCC[C@H](Nc4c(F)cc(S(C)(=O)=O)cc4F)C3=O)CC2)no1]

Step A:

To a solution of (S)-3-(2,6-difluoro-4-(methylsulfonyl)phenylamino)-1,4′-bipiperidin-2-one (Preparation F-2; 0.94 g, 2.4 mmol) in acetonitrile (100 mL) was added potassium carbonate (0.70 g, 5.1 mmol) and cyanic bromide (5 M in acetonitrile, 0.58 mL, 2.9 mmol) and the reaction stirred overnight at ambient temperature. The reaction was next made basic with 1 N NaOH solution. The solution was extracted with EtOAc and the organic layer was washed with 1 N NaOH. The organic layer was dried over MgSO₄ and concentrated to give (S)-3-(2,6-difluoro-4-(methylsulfonyl)phenylamino)-2-oxo-1,4′-bipiperidine-1′-carbonitrile (1.0 g, 100%) as a white solid.

Step B:

To a solution of (S)-3-(2,6-difluoro-4-(methylsulfonyl)phenylamino)-2-oxo-1,4′-bipiperidine-1′-carbonitrile (0.18 g, 0.43 mmol) in EtOH (4 mL) was added hydroxylamine (0.057 g, 0.858 mmol, 50% in water) and the reaction stirred in a sealed vessel at 60° C. overnight. The solution was cooled and concentrated under vacuum to give crude (S)-3-(2,6-difluoro-4-(methylsulfonyl)phenylamino)-N-hydroxy-2-oxo-1,4′-bipiperidine-1′-carboximidamide (0.19 g, 0.42 mmol, 98% yield) as a white solid. Mass spectrum (apci) m/z=446.2 (M+H).

Step C:

A solution of (S)-3-(2,6-difluoro-4-(methylsulfonyl)phenylamino)-N-hydroxy-2-oxo-1,4′-bipiperidine-1′-carboximidamide (0.11 g, 0.25 mmol) and isobutyric anhydride (0.039 g, 0.25 mmol) in dioxane (2 mL). The reaction mixture was heated in a sealed tube at 130° C. for 20 minutes. The solution was cooled and concentrated under vacuum. Reverse phase HPLC gave (S)-3-(2,6-difluoro-4-(methylsulfonyl)phenylamino)-1′-(5-isopropyl-1,2,4-oxadiazol-3-yl)-1,4′-bipiperidin-2-one (0.044 g, 0.088 mmol, 35% yield) as a white solid. Mass spectrum (apci) m/z=498.1 (M+H).

### Example 13

### (S)-3-(2-fluoro-4-(methylsulfonyl)phenylamino)-1-(1-(5-(trichloromethyl)-1,2,4-oxadiazol-3-yl)piperidin-4-yl)pyrrolidin-2-one

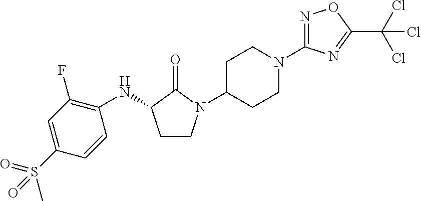 [SMILES: CS(=O)(=O)c1ccc(N[C@H]2CCN(C3CCN(c4noc(C(Cl)(Cl)Cl)n4)CC3)C2=O)c(F)c1]

Prepared according to the method of Example 12, using (S)-3-(2-fluoro-4-(methylsulfonyl)phenylamino)-1-(piperidin-4-yl)pyrrolidin-2-one (Preparation C) in Step A and using 2,2,2-trichloroacetic anhydride in Step C. ¹H NMR (400 MHz, CDCl₃): δ 1.75-1.98 (m, 5H), 2.70-2.80 (m, 1H), 3.05 (s, 3H), 2.10-3.18 (m, 2H), 3.38-3.48 (m, 2H), 4.12-4.32 (m, 4H), 5.06 (t, 1H), 6.78 (t, 1H), 7.54 (dd, 1H), 7.58 (d, 1H).

### Example 14

### (S)-3-(2-fluoro-4-(methylsulfonyl)phenylamino)-1′-(5-isopropyl-1,2,4-oxadiazol-3-yl)-1,4′-bipiperidin-2-one

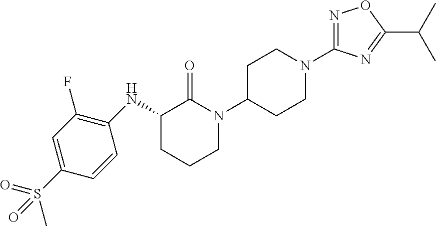 [SMILES: CC(C)c1nc(N2CCC(N3CCC[C@H](Nc4ccc(S(C)(=O)=O)cc4F)C3=O)CC2)no1]

Prepared according to the method of Example 12, using (S)-3-(2-fluoro-4-(methylsulfonyl)phenylamino)-1,4′-bipiperidin-2-one hydrochloride (Preparation E) and 2 equivalents of base in Step A. Mass spectrum (apci) m/z=480.2 (M+H).

### Example 15

### (S)-3-(2-fluoro-4-(methylsulfonyl)phenylamino)-1-(1-(5-isopropyl-1,2,4-oxadiazol-3-yl)piperidin-4-yl)pyrrolidin-2-one

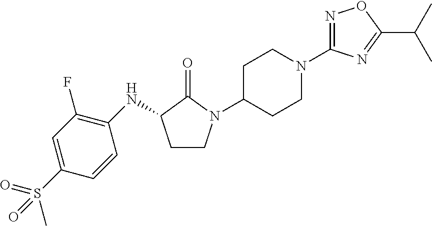 [SMILES: CC(C)c1nc(N2CCC(N3CC[C@H](Nc4ccc(S(C)(=O)=O)cc4F)C3=O)CC2)no1]

Prepared according to the method of Example 12, using (S)-3-(2-fluoro-4-(methylsulfonyl)phenylamino)-1-(piperidin-4-yl)pyrrolidin-2-one (Preparation C) in Step A. Mass spectrum (apci) m/z=466.2 (M+H).

### Example 16

### (S)-3-(2-fluoro-4-(methylsulfonyl)phenylamino)-1-(1-(5-(trifluoromethyl)-1,2,4-oxadiazol-3-yl)piperidin-4-yl)pyrrolidin-2-one

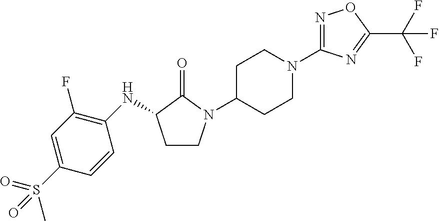 [SMILES: CS(=O)(=O)c1ccc(N[C@H]2CCN(C3CCN(c4noc(C(F)(F)F)n4)CC3)C2=O)c(F)c1]

Prepared according to the method of Example 12, using (S)-3-(2-fluoro-4-(methylsulfonyl)phenylamino)-1-(piperidin-4-yl)pyrrolidin-2-one (Preparation C) in Step A and using 2,2,2-trifluoroacetic anhydride in Step C. ¹H NMR (400 MHz, CDCl₃): δ 1.70-1.98 (m, 5H), 2.73-2.82 (m, 1H), 3.02 (s, 3H), 3.05-3.20 (m, 2H), 3.35-3.45 (m, 2H), 4.10-4.22 (m, 3H), 4.22-4.35 (m, 1H), 5.04 (bs, 1H), 6.80 (t, 1H), 7.58 (d, 1H), 7.61 (d, 1H).

### Example 17

### (S)-3-(2,5-difluoro-4-(methylsulfonyl)phenoxy)-1-(1-(5-phenyl-1,2,4-oxadiazol-3-yl)piperidin-4-yl)pyrrolidin-2-one

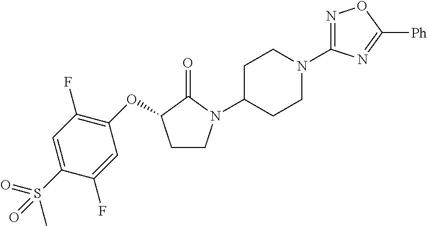 [SMILES: CS(=O)(=O)c1cc(F)c(O[C@H]2CCN(C3CCN(c4noc(-c5ccccc5)n4)CC3)C2=O)cc1F]

Prepared according to the method of Example 12, using (S)-3-(2,5-difluoro-4-(methylsulfonyl)phenoxy)-1-(piperidin-4-yl)pyrrolidin-2-one (Preparation F-4) in Step A and using benzoic anhydride in Step C. Mass spectrum (apci) m/z=519.1 (M+H).

### Example 18

### (S)-3-(2-fluoro-4-(methylsulfonyl)phenoxy)-1-(1-(5-isopropyl-1,2,4-oxadiazol-3-yl)piperidin-4-yl)pyrrolidin-2-one

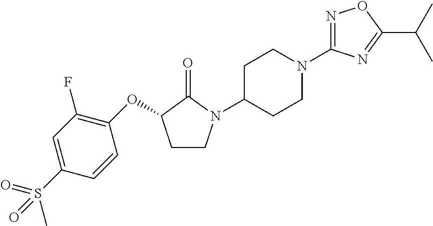 [SMILES: CC(C)c1nc(N2CCC(N3CC[C@H](Oc4ccc(S(C)(=O)=O)cc4F)C3=O)CC2)no1]

Prepared according to the method of Example 12, using (S)-3-(2-fluoro-4-(methylsulfonyl)phenoxy)-1-(piperidin-4-yl)pyrrolidin-2-one (Preparation F-3) in Step A. Mass spectrum (apci) m/z=467.1 (M+H).

### Example 19

### (S)-3-(2,5-difluoro-4-(methylsulfonyl)phenoxy)-1-(1-(5-isopropyl-1,2,4-oxadiazol-3-yl)piperidin-4-yl)pyrrolidin-2-one

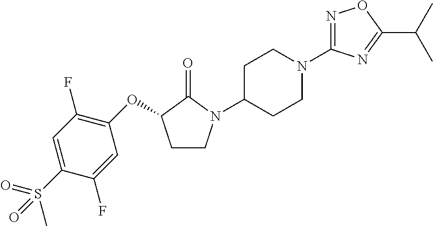 [SMILES: CC(C)c1nc(N2CCC(N3CC[C@H](Oc4cc(F)c(S(C)(=O)=O)cc4F)C3=O)CC2)no1]

Prepared according to the method of Example 12, using (S)-3-(2,5-difluoro-4-(methylsulfonyl)phenoxy)-1-(piperidin-4-yl)pyrrolidin-2-one (Preparation F-4) in Step A. Mass spectrum (apci) m/z=485.2 (M+H).

### Example 20

### (S)-3-(2,6-difluoro-4-(methylsulfonyl)phenoxy)-1-(1-(5-isopropyl-1,2,4-oxadiazol-3-yl)piperidin-4-yl)pyrrolidin-2-one

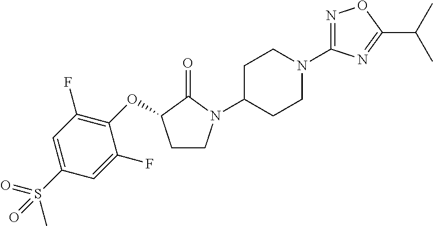 [SMILES: CC(C)c1nc(N2CCC(N3CC[C@H](Oc4c(F)cc(S(C)(=O)=O)cc4F)C3=O)CC2)no1]

Prepared according to the method of Example 12, using (S)-3-(2,6-difluoro-4-(methylsulfonyl)phenoxy)-1-(piperidin-4-yl)pyrrolidin-2-one hydrochloride (Preparation D-2) in Step A. Mass spectrum (apci) m/z=485.1 (M+H).

### Example 21

### (S)-1-(1-(5-(difluoromethyl)-1,2,4-oxadiazol-3-yl)piperidin-4-yl)-3-(2-fluoro-4-(methylsulfonyl)phenylamino)pyrrolidin-2-one

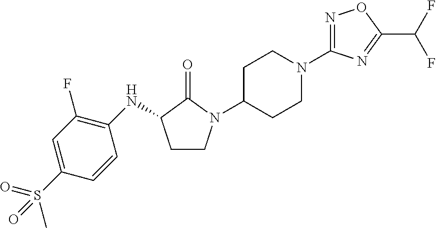 [SMILES: CS(=O)(=O)c1ccc(N[C@H]2CCN(C3CCN(c4noc(C(F)F)n4)CC3)C2=O)c(F)c1]

Step A:

To a solution of cyanic bromide (0.14 g, 1.4 mmol) in acetonitrile (30 mL) was added potassium carbonate (0.31 g, 2.3 mmol) and (S)-3-(2-fluoro-4-(methylsulfonyl)phenylamino)-1-(piperidin-4-yl)pyrrolidin-2-one (Preparation C, 0.40 g, 1.1 mmol) and the reaction was stirred for 90 minutes at ambient temperature. The reaction was quenched with water and made basic with 1 N NaOH solution. EtOAc was added (100 mL) and the organic layer was separated and washed with 1 N NaOH solution, brine, dried over MgSO₄ and concentrated to give (S)-4-(3-(2-fluoro-4-(methylsulfonyl)phenylamino)-2-oxopyrrolidin-1-yl)piperidine-1-carbonitrile (0.32 g, 0.844 mmol, 75%) as a white solid. Mass spectrum (apci) m/z=381.1 (M+H).

Step B:

To a solution of (S)-4-(3-(2-fluoro-4-(methylsulfonyl)phenylamino)-2-oxopyrrolidin-1-yl)piperidine-1-carbonitrile (0.92 g, 2.4 mmol) in EtOH (35 mL) was added hydroxylamine (0.32 g, 4.8 mmol, 50% in water) and the reaction was stirred at 60° C. overnight. The solution was cooled and concentrated to give the (S)-4-(3-(2-fluoro-4-(methylsulfonyl)phenylamino)-2-oxopyrrolidin-1-yl)-N-hydroxypiperidine-1-carboximidamide (0.98 g, 2.4 mmol, 98% yield) as a white solid. Mass spectrum (apci) m/z=414.2 (M+H).

Step C:

(S)-4-(3-(2-fluoro-4-(methylsulfonyl)phenylamino)-2-oxopyrrolidin-1-yl)-N-hydroxypiperidine-1-carboximidamide (0.10 g, 0.24 mmol) and 2,2-difluoroacetic anhydride (0.042 g, 0.24 mmol) were combined and charged with dioxane (2 mL). The solution was heated in a sealed tube at 90° C. for 4 hours. The solution was cooled and concentrated, and the residue was diluted with water, extracted with EtOAc, dried and concentrated. Flash chromatography of the crude material gave (S)-1-(1-(5-(difluoromethyl)-1,2,4-oxadiazol-3-yl)piperidin-4-yl)-3-(2-fluoro-4-(methylsulfonyl)phenylamino)pyrrolidin-2-one (0.032 g, 0.068 mmol, 28% yield) as a white solid. ¹H NMR (400 MHz, CDCl₃): δ 1.72-1.98 (m, 5H), 2.73-2.82 (m, 1H), 3.01 (s, 3H), 3.05-3.15 (m, 2H), 3.38-3.48 (m, 2H), 4.10-4.23 (m, 3H), 4.23-4.32 (m, 1H), 5.08 (bs, 1H), 6.63 (t, 1H), 6.80 (t, 1H), 7.55 (d, 1H), 7.61 (d, 1H). (MS DATA???)

### Example 22

### (S)-3-(2,6-difluoro-4-(methylsulfonyl)phenylamino)-1-(1-(5-(trifluoromethyl)-1,2,4-oxadiazol-3-yl)piperidin-4-yl)pyrrolidin-2-one

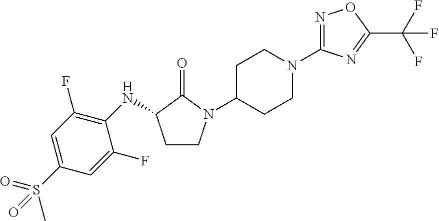 [SMILES: CS(=O)(=O)c1cc(F)c(N[C@H]2CCN(C3CCN(c4noc(C(F)(F)F)n4)CC3)C2=O)c(F)c1]

Prepared according to the method of Example 12, using (S)-3-(2,6-difluoro-4-(methylsulfonyl)phenylamino)-1-(piperidin-4-yl)pyrrolidin-2-one (Preparation C-2) in Step A and using 2,2,2-trifluoroacetic anhydride in Step C. ¹H NMR (400 MHz, CDCl₃): δ 1.70-1.88 (m, 4H), 1.88-2.0 (m, 1H), 2.70-2.79 (m, 1H), 3.02 (s, 3H), 3.02-3.18 (m, 2H), 3.30-3.42 (m, 2H), 4.12-4.30 (m, 3H), 4.42-4.50 (m, 1H), 4.72-4.82 (m, 1H) 7.42 (d, 2H).

### Example 23

### (S)-3-(2,5-difluoro-4-(methylsulfonyl)phenylamino)-1-(1-(5-(trifluoromethyl)-1,2,4-oxadiazol-3-yl)piperidin-4-yl)pyrrolidin-2-one

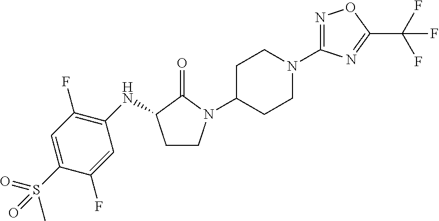 [SMILES: CS(=O)(=O)c1cc(F)c(N[C@H]2CCN(C3CCN(c4noc(C(F)(F)F)n4)CC3)C2=O)cc1F]

Prepared according to the method of Example 12, using (S)-3-(2,5-difluoro-4-(methylsulfonyl)phenylamino)-1-(piperidin-4-yl)pyrrolidin-2-one (Preparation C-1) in Step A and using 2,2,2-trifluoroacetic anhydride in Step C. ¹H NMR (400 MHz, CDCl₃): δ 1.72-1.98 (m, 5H), 2.72-2.81 (m, 1H), 3.07-3.20 (m, 2H), 3.18 (s, 3H), 3.35-3.49 (m, 2H), 4.07-4.13 (m, 1H), 4.15-4.23 (m, 2H), 4.23-4.31 (m, 1H), 5.12 (bs, 1H), 6.50 (dd, 1H), 7.52 (dd, 1H).

### Example 24

### (S)-3-(2-fluoro-4-(methylsulfonyl)phenoxy)-1-(1-(5-(trifluoromethyl)-1,2,4-oxadiazol-3-yl)piperidin-4-yl)pyrrolidin-2-one

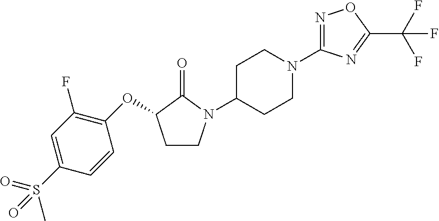 [SMILES: CS(=O)(=O)c1ccc(O[C@H]2CCN(C3CCN(c4noc(C(F)(F)F)n4)CC3)C2=O)c(F)c1]

Prepared according to the method of Example 12, using (S)-3-(2-fluoro-4-(methylsulfonyl)phenylamino)-1-(piperidin-4-yl)pyrrolidin-2-one (Preparation C) in Step A and using 2,2,2-trifluoroacetic anhydride in Step C. ¹H NMR (400 MHz, CDCl₃): δ 1.72-1.90 (m, 4H), 2.28-2.39 (m, 1H), 2.55-2.63 (m, 1H), 3.02 (s, 3H), 3.02-3.12 (m, 2H), 3.35-3.41 (m, 1H), 3.51-3.58 (m, 1H), 4.13-4.30 (m, 3H), 5.02 (t, 1H), 7.52 (t, 1H), 7.68 (t, 2H).

### Example 25

### (S)-3-(2,5-difluoro-4-(methylsulfonyl)phenylamino)-1′-(5-(trifluoromethyl)-1,2,4-oxadiazol-3-yl)-1,4′-bipiperidin-2-one

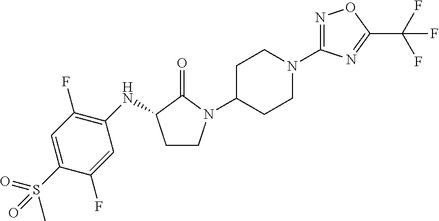 [SMILES: CS(=O)(=O)c1cc(F)c(N[C@H]2CCN(C3CCN(c4noc(C(F)(F)F)n4)CC3)C2=O)cc1F]

Prepared according to the method of Example 12, using (S)-3-(2,5-difluoro-4-(methylsulfonyl)phenylamino)-1,4′-bipiperidin-2-one (Preparation F-1) in Step A and using 2,2,2-trifluoroacetic anhydride in Step C. Mass spectrum (apci) m/z=524 (M+H).

### Example 26

### (S)-3-(2,6-difluoro-4-(methylsulfonyl)phenylamino)-1′-(5-(trifluoromethyl)-1,2,4-oxadiazol-3-yl)-1,4′-bipiperidin-2-one

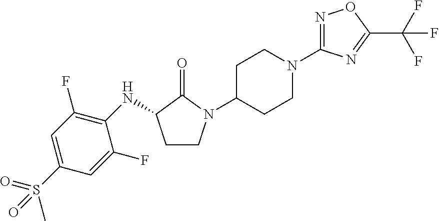 [SMILES: CS(=O)(=O)c1cc(F)c(N[C@H]2CCN(C3CCN(c4noc(C(F)(F)F)n4)CC3)C2=O)c(F)c1]

Step A:

To a solution of cyanic bromide (0.58 mL, 2.9 mmol) in acetonitrile (100 mL) was added potassium carbonate (0.70 g, 5.1 mmol) and (S)-3-(2,6-difluoro-4-(methylsulfonyl)phenylamino)-1,4′-bipiperidin-2-one (Preparation F-2; 0.94 g, 2.4 mmol) and the reaction stirred overnight at ambient temperature. The reaction was quenched with water and made basic with 1 N NaOH solution. EtOAc was added (100 mL) and the organic layer was separated and washed with 1 N NaOH solution, brine, dried over MgSO₄ and concentrated to give (S)-3-(2,6-difluoro-4-(methylsulfonyl)phenylamino)-2-oxo-1,4′-bipiperidine-1′-carbonitrile (1.04 g, 2.5 mmol, 100%) as a white solid.

Step B:

To a solution of (S)-3-(2,6-difluoro-4-(methylsulfonyl)phenylamino)-2-oxo-1,4′-bipiperidine-1′-carbonitrile (0.50 g, 1.2 mmol) in EtOH (10 mL) was added hydroxylamine (0.16 g, 2.4 mmol, 50% in water) and the reaction stirred at 60° C. overnight. The solution was cooled and concentrated to give (S)-3-(2,6-difluoro-4-(methylsulfonyl)phenylamino)-N-hydroxy-2-oxo-1,4′-bipiperidine-1′-carboximidamide (0.56 g, 1.3 mmol, 100%) as a white solid.

Step C:

(S)-3-(2,6-difluoro-4-(methylsulfonyl)phenylamino)-N-hydroxy-2-oxo-1,4′-bipiperidine-1′-carboximidamide (0.25 g, 0.56 mmol) and 2,2,2-trifluoroacetic anhydride (0.12 g, 0.56 mmol) were combined with dioxane (6 mL). The solution was heated in a sealed tube at 30° C. for 4 hours and then at 60° C. overnight. The solution was cooled and concentrated. The crude material was purified by reverse phase HPLC to provide (S)-3-(2,6-difluoro-4-(methylsulfonyl)phenylamino)-1′-(5-(trifluoromethyl)-1,2,4-oxadiazol-3-yl)-1,4′-bipiperidin-2-one (0.039 g, 0.075 mmol, 13%) as a white solid. ¹H NMR: (400 MHz, CDCl₃): δ 1.60-1.70 (m, 1H), 1.72-1.83 (m, 4H), 1.95-2.03 (m, 2H), 2.42-2.50 (m, 1H), 3.02 (s, 3H), 3.08-3.18 (m, 2H), 3.32 (t, 2H), 4.13-4.20 (m, 2H), 4.30-4.39 (m, 1H), 4.61-4.72 (m, 1H), 5.12-5.18 (m, 1H), 7.42 (d, 2H).

### Example 27

### (S)-3-(2,5-difluoro-4-(methylsulfonyl)phenoxy)-1-(1-(5-(trifluoromethyl)-1,2,4-oxadiazol-3-yl)piperidin-4-yl)pyrrolidin-2-one

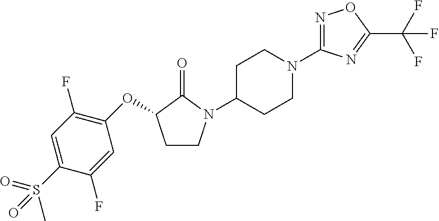 [SMILES: CS(=O)(=O)c1cc(F)c(O[C@H]2CCN(C3CCN(c4noc(C(F)(F)F)n4)CC3)C2=O)cc1F]

Step A:

To a solution of (S)-3-(2,5-difluoro-4-(methylsulfonyl)phenoxy)-1-(piperidin-4-yl)pyrrolidin-2-one (Preparation F-4; 1.4 g, 3.6 mmol) in acetonitrile (100 mL) was added potassium carbonate (1.05 g, 7.6 mmol) and cyanic bromide (0.87 mL, 4.4 mmol, 5 M in acetonitrile) and the reaction was stirred overnight at ambient temperature. The reaction was quenched with 1 N NaOH solution and extracted with ethyl acetate (300 mL). The combined organic layers were washed with 1 N NaOH solution, brine, dried over MgSO₄, and concentrated to give (S)-4-(3-(2,5-difluoro-4-(methylsulfonyl)phenoxy)-2-oxopyrrolidin-1-yl)piperidine-1-carbonitrile (1.4 g, 3.5 mmol, 97%) as a white solid.

Step B:

To a solution of (S)-4-(3-(2,5-difluoro-4-(methylsulfonyl)phenoxy)-2-oxopyrrolidin-1-yl)piperidine-1-carbonitrile (0.36 g, 0.90 mmol) in EtOH (10 mL) was added hydroxylamine (0.12 g, 1.8 mmol) and the reaction was stirred at 60° C. overnight. The solution was cooled and concentrated to give (S)-4-(3-(2,5-difluoro-4-(methylsulfonyl)phenoxy)-2-oxopyrrolidin-1-yl)-N-hydroxypiperidine-1-carboximidamide (0.37 g, 0.86 mmol, 95% yield) as a white solid. Mass spectrum (apci) m/z=433.1 (M+H).

Step C:

(S)-4-(3-(2,5-Difluoro-4-(methylsulfonyl)phenoxy)-2-oxopyrrolidin-1-yl)-N-hydroxypiperidine-1-carboximidamide (0.20 g, 0.46 mmol) and 2,2,2-trifluoroacetic anhydride (0.10 g, 0.49 mmol) were combined with dioxane (6 mL). The solution was heated in a sealed tube at 30° C. for 1 hour and then at 90° C. for 3 hours. The solution was cooled and concentrated. Purification of the crude material by reverse phase HPLC gave (S)-3-(2,5-difluoro-4-(methylsulfonyl)phenoxy)-1-(1-(5-(trifluoromethyl)-1,2,4-oxadiazol-3-yl)piperidin-4-yl)pyrrolidin-2-one (0.055 g, 0.11 mmol, 23% yield) as a white solid. ¹H NMR (400 MHz, CDCl₃): δ 1.72-1.90 (m, 4H), 2.30-2.40 (m, 1H), 2.57-2.65 (m, 1H), 3.08-3.15 (m, 2H), 3.20 (s, 3H), 3.35-3.42 (m, 1H), 3.51-3.58 (m, 1H), 4.13-4.27 (m, 3H), 4.98 (t, 1H), 7.35 (dd, 1H), 7.67 (dd, 1H).

### Example 28

### (S)-3-(2,6-difluoro-4-(methylsulfonyl)phenoxy)-1-(1-(5-(trifluoromethyl)-1,2,4-oxadiazol-3-yl)piperidin-4-yl)pyrrolidin-2-one

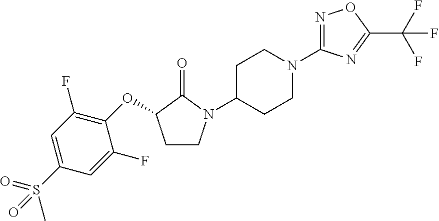 [SMILES: CS(=O)(=O)c1cc(F)c(O[C@H]2CCN(C3CCN(c4noc(C(F)(F)F)n4)CC3)C2=O)c(F)c1]

Prepared according to the method of Example 12, using (S)-3-(2,6-difluoro-4-(methylsulfonyl)phenoxy)-1-(piperidin-4-yl)pyrrolidin-2-one hydrochloride (Preparation D-2) in Step A and using 2,2,2-trifluoroacetic anhydride in Step C. ¹H NMR (400 MHz, CDCl₃): δ 1.72-1.90 (m, 4H), 2.31-2.40 (m, 1H), 2.52-2.60 (m, 1H), 3.02-3.15 (m, 2H), 3.05 (s, 3H), 3.31-3.38 (m, 1H), 3.51-3.60 (m, 1H), 4.11-4.23 (m, 3H), 5.02 (t, 1H), 7.53 (d, 2H).

### Example 29

### (S)-3-(2-fluoro-4-(methylsulfonyl)phenylamino)-1′-(5-(trifluoromethyl)-1,2,4-oxadiazol-3-yl)-1,4′-bipiperidin-2-one

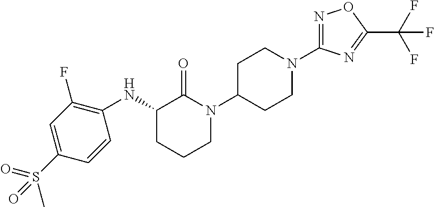 [SMILES: CS(=O)(=O)c1ccc(N[C@H]2CCCN(C3CCN(c4noc(C(F)(F)F)n4)CC3)C2=O)c(F)c1]

Prepared according to the method of Example 12, using (S)-3-(2-fluoro-4-(methylsulfonyl)phenylamino)-1,4′-bipiperidin-2-one hydrochloride (Preparation E) and 2 equivalents of base in Step A and using 2,2,2-trifluoroacetic anhydride in Step C. ¹H NMR (400 MHz, CDCl₃): δ 1.60-1.70 (m, 1H), 1.70-1.85 (5H), 1.95-2.03 (m, 2H), 2.48-2.53 (m, 1H), 3.02 (s, 3H), 3.07-3.15 (m, 2H), 3.33 (t, 2H), 3.97-4.03 (m, 1H), 4.12-4.20 (m, 2H), 5.50 (bs, 1H), 6.75 (t, 1H), 7.53 (d, 1H), 7.60 (d, 1H).

### Example 30

### (S)-3-(2,5-difluoro-4-(methylsulfonyl)phenylamino)-1-(1-(5-(difluoromethyl)-1,2,4-oxadiazol-3-yl)piperidin-4-yl)pyrrolidin-2-one

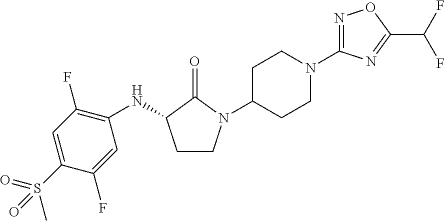 [SMILES: CS(=O)(=O)c1cc(F)c(N[C@H]2CCN(C3CCN(c4noc(C(F)F)n4)CC3)C2=O)cc1F]

Prepared according to the method of Example 12, using (S)-3-(2,5-difluoro-4-(methylsulfonyl)phenylamino)-1-(piperidin-4-yl)pyrrolidin-2-one (Preparation C-1) in Step A and using 2,2-difluoroacetic anhydride in Step C. ¹H NMR (400 MHz, CDCl₃): δ 1.72-1.88 (m, 4H), 1.88-1.98 (m, 1H), 2.70-2.80 (m, 1H), 3.02-3.18 (m, 2H), 3.08 (m, 3H), 3.33-3.49 (m, 2H), 4.07-4.12 (m, 1H), 4.15-4.22 (m, 2H), 4.22-4.30 (m, 1H), 5.15 (bs, 1H), 6.50 (dd, 1H), 6.65 (t, 1H), 7.52 (dd, 1H).

### Example 31

### (S)-3-(2,6-difluoro-4-(methylsulfonyl)phenylamino)-1-(1-(5-(difluoromethyl)-1,2,4-oxadiazol-3-yl)piperidin-4-yl)pyrrolidin-2-one

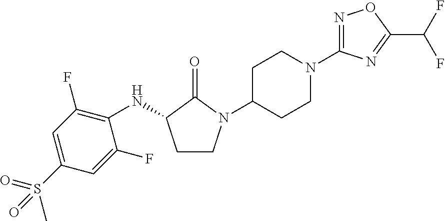 [SMILES: CS(=O)(=O)c1cc(F)c(N[C@H]2CCN(C3CCN(c4noc(C(F)F)n4)CC3)C2=O)c(F)c1]

Step A:

To a solution of cyanic bromide (0.96 mL, 4.8 mmol) in acetonitrile (160 mL) was added potassium carbonate (1.17 g, 8.4 mmol) and (S)-3-(2,6-difluoro-4-(methylsulfonyl)phenylamino)-1-(piperidin-4-yl)pyrrolidin-2-one (Preparation C-2; 1.5 g, 4.0 mmol) and the reaction was stirred overnight at ambient temperature. The reaction was quenched with 1 N NaOH solution and extracted with ethyl acetate (300 mL). The combined organic layers were washed with 1 N NaOH solution, brine, dried over MgSO₄, and concentrated to give (S)-4-(3-(2,6-difluoro-4-(methylsulfonyl)phenylamino)-2-oxopyrrolidin-1-yl)piperidine-1-carbonitrile (1.7 g, 4.1 mmol, 100%) as a white solid.

Step B:

To a solution of (S)-4-(3-(2,6-difluoro-4-(methylsulfonyl)phenylamino)-2-oxopyrrolidin-1-yl)piperidine-1-carbonitrile (1.7 g, 4.1 mmol) in EtOH (35 mL) was added hydroxylamine (0.55 g, 8.3 mmol) and the reaction was stirred at 60° C. overnight. The solution was cooled and concentrated to give the (S)-4-(3-(2,6-difluoro-4-(methylsulfonyl)phenylamino)-2-oxopyrrolidin-1-yl)-N-hydroxypiperidine-1-carboximidamide (1.75 g, 4.1 mmol, 98% yield) as a white solid. Mass spectrum (apci) m/z=432.1 (M+H).

Step C:

(S)-4-(3-(2,6-difluoro-4-(methylsulfonyl)phenylamino)-2-oxopyrrolidin-1-yl)-N-hydroxypiperidine-1-carboximidamide in dioxane (4 mL) was charged with 2,2-difluoroacetic anhydride (0.097 g, 0.56 mmol). The mixture was stirred at 60° C. overnight. The solution was concentrated and the crude material was purified by flash chromatography (80-100% EtOAc/hexanes) to give (S)-3-(2,6-difluoro-4-(methylsulfonyl)phenylamino)-1-(1-(5-(difluoromethyl)-1,2,4-oxadiazol-3-yl)piperidin-4-yl)pyrrolidin-2-one (0.16 g, 0.33 mmol, 70% yield) as a white solid. ¹H NMR (400 MHz, CDCl₃): δ 1.72-1.88 (m, 4H), 1.90-2.00 (m, 1H), 2.70-2.79 (m, 1H), 3.02 (s, 3H), 3.02-3.15 (m, 2H), 3.30-3.46 (m, 2H), 4.12-4.29 (m, 3H), 4.12-4.30 (m, 1H), 4.75-4.83 (m, 1H), 6.64 (t, 1H), 7.43 (d, 2H).

### Example 32

### (S)-3-(2,6-difluoro-4-(methylsulfonyl)phenylamino)-1′-(5-(difluoromethyl)-1,2,4-oxadiazol-3-yl)-1,4′-bipiperidin-2-one

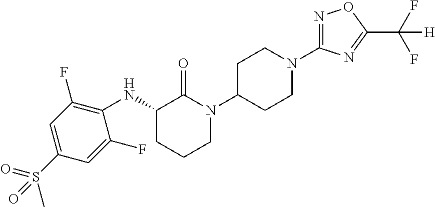 [SMILES: CS(=O)(=O)c1cc(F)c(N[C@H]2CCCN(C3CCN(c4noc(C(F)F)n4)CC3)C2=O)c(F)c1]

Prepared according to the method of Example 12, using (S)-3-(2,6-difluoro-4-(methylsulfonyl)phenylamino)-1,4′-bipiperidin-2-one (Preparation F-2) in Step A and using 2,2-difluoroacetic anhydride in Step C. ¹H NMR 400 MHz, CDCl₃): δ 1.59-1.69 (m, 1H), 1.70-1.82 (m, 4H), 1.91-2.00 (m, 2H), 2.43-2.51 (m, 1H), 3.02 (s, 3H), 3.00-3.10 (m, 2H), 3.29 (t, 2H), 4.12-4.20 (m, 2H), 4.30-4.38 (m, 1H), 4.61-4.71 (m, 1H), 5.12-5.20 (m, 1H), 6.62 (t, 1H), 7.42 (d, 2H).

### Example 33

### (S)-3-(2,5-difluoro-4-(methylsulfonyl)phenylamino)-1′-(5-(difluoromethyl)-1,2,4-oxadiazol-3-yl)-1,4′-bipiperidin-2-one

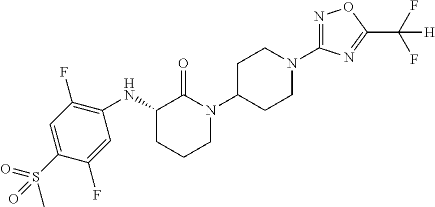 [SMILES: CS(=O)(=O)c1cc(F)c(N[C@H]2CCCN(C3CCN(c4noc(C(F)F)n4)CC3)C2=O)cc1F]

Prepared according to the method of Example 12, using (S)-3-(2,5-difluoro-4-(methylsulfonyl)phenylamino)-1,4′-bipiperidin-2-one (Preparation F-1) in Step A and using 2,2-difluoroacetic anhydride in Step C. ¹H NMR 400 MHz, CDCl₃): δ 1.58-1.70 (m, 1H), 1.71-1.83 (m, 4H), 1.95-2.03 (m, 2H), 2.41-2.51 (m, 1H), 3.02-3.12 (m, 2H), 3.17 (s, 3H), 3.32 (t, 2H), 3.90-3.97 (m, 1H), 4.12-4.20 (m, 2H), 4.63-4.71 (m, 1H), 5.09 (bs, 1H), 6.45 (dd, 1H), 6.63 (t, 1H), 7.50 (dd, 1H).

### Example 34

### (S)-3-(4-(ethylsulfonyl)-2-fluorophenylamino)-1-(1-(5-(trifluoromethyl)-1,2,4-oxadiazol-3-yl)piperidin-4-yl)pyrrolidin-2-one

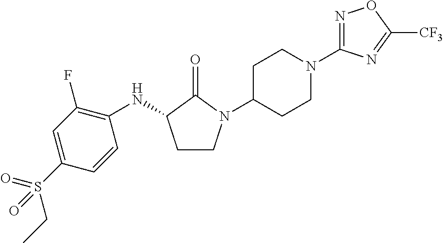 [SMILES: CCS(=O)(=O)c1ccc(N[C@H]2CCN(C3CCN(c4noc(C(F)(F)F)n4)CC3)C2=O)c(F)c1]

Prepared according to the method of Example 12, using (S)-3-(4-(ethylsulfonyl)-2-fluorophenylamino)-1-(piperidin-4-yl)pyrrolidin-2-one (Preparation C-3) in Step A and using 2,2,2-trifluoroacetic anhydride in Step C. ¹H NMR 400 MHz, CDCl₃): δ 1.28 (t, 3H), 1.72-1.98 (m, 5H), 2.72-2.80 (m, 1H), 3.10 (q, 2H), 3.02-3.18 (m, 2H), 3.33-3.51 (m, 2H), 4.10-4.22 (m, 3H), 4.22-4.32 (m, 1H), 5.07 (bs, 1H), 6.69 (t, 1H), 7.51 (dd, 2H).

### Example 35

### (S)-3-(2,5-difluoro-4-(methylsulfonyl)phenylamino)-1′-(5-(difluoromethyl)-1,2,4-oxadiazol-3-yl)-1,4′-bipiperidin-2-one

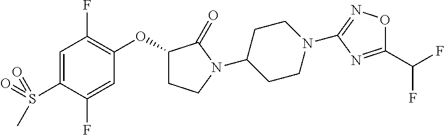 [SMILES: CS(=O)(=O)c1cc(F)c(O[C@H]2CCN(C3CCN(c4noc(C(F)F)n4)CC3)C2=O)cc1F]

Prepared according to the method of Example 12, using (S)-3-(2,5-difluoro-4-(methylsulfonyl)phenoxy)-1-(piperidin-4-yl)pyrrolidin-2-one (Preparation F-4) in Step A and using 2,2-difluoroacetic anhydride in Step C. ¹H NMR 400 MHz, CDCl₃): δ 1.72-1.87 (m, 4H), 2.20-2.48 (m, 1H), 2.53-2.65 (m, 1H), 3.02-3.14 (m, 2H), 3.19 (s, 3H), 3.35-3.42 (m, 1H), 3.51-3.59 (m, 1H), 4.15-4.30 (m, 3H), 4.99 (t, 1H), 6.63 (t, 1H), 7.35 (dd, 1H), 7.65 (dd, 1H).

### Example 36

### (S)-3-(2-fluoro-4-(methylsulfonyl)phenylamino)-1-(1-(5-(2,2,2-trifluoroethyl)-1,2,4-oxadiazol-3-yl)piperidin-4-yl)pyrrolidin-2-one

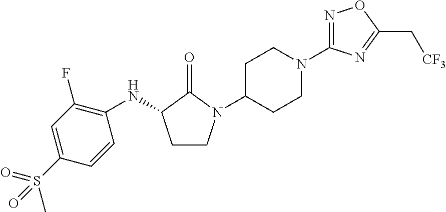 [SMILES: CS(=O)(=O)c1ccc(N[C@H]2CCN(C3CCN(c4noc(CC(F)(F)F)n4)CC3)C2=O)c(F)c1]

Step A:

To a solution of (S)-3-(2-fluoro-4-(methylsulfonyl)phenylamino)-1-(piperidin-4-yl)pyrrolidin-2-one (Preparation C, 0.95 g, 2.7 mmol) in acetonitrile (60 mL) was added potassium carbonate (0.78 g, 5.6 mmol) and cyanic bromide (5 M in acetonitrile, 0.64 mL, 3.2 mmol) and the reaction stirred overnight at ambient temperature. The reaction was next made basic with 1 N NaOH solution. The material was then extracted with EtOAc. The organic layer was washed with 1 N NaOH, dried over MgSO₄ and concentrated to give (S)-4-(3-(2-fluoro-4-(methylsulfonyl)phenylamino)-2-oxopyrrolidin-1-yl)piperidine-1-carbonitrile (0.92 g, 90%) as a white solid.

Step B:

To a solution of (S)-4-(3-(2-fluoro-4-(methylsulfonyl)phenylamino)-2-oxopyrrolidin-1-yl)piperidine-1-carbonitrile (0.92 g, 2.4 mmol) in EtOH (35 mL) was added hydroxylamine (50% in water, 0.32 g, 4.8 mmol) and the reaction stirred at 60° C. overnight. The solution was cooled and concentrated to give (S)-4-(3-(2-fluoro-4-(methylsulfonyl)phenylamino)-2-oxopyrrolidin-1-yl)-N-hydroxypiperidine-1-carboximidamide (0.98 g, 2.4 mmol, 98% yield) as a white solid; MS (apci) m/z=414.2 (M+H).

Step C:

To 3,3,3-trifluoropropanoic acid (0.031 g, 0.24 mmol) in DMF (2 mL) was added diisopropylethylamine (0.042 mL, 0.24 mmol) and N-((dimethylamino)fluoromethylene)-N-methylmethanaminium hexafluorophosphate(V) (0.064 g, 0.24 mmol). The reaction was stirred at ambient temperature for 30 minutes, and then (S)-4-(3-(2-fluoro-4-(methylsulfonyl)phenylamino)-2-oxopyrrolidin-1-yl)-N-hydroxypiperidine-1-carboximidamide (0.10 g, 0.24 mmol) was added. The reaction was stirred at 110° C. for 3 hours. The solution was diluted with saturated NaHCO₃ and extracted with EtOAc. The organic layer was dried over MgSO₄ and concentrated under vacuum. Reverse phase HPLC purification gave (S)-3-(2-fluoro-4-(methylsulfonyl)phenylamino)-1-(1-(5-(2,2,2-trifluoroethyl)-1,2,4-oxadiazol-3-yl)piperidin-4-yl)pyrrolidin-2-one (0.046 g, 0.091 mmol, 38% yield) as a white solid. Mass spectrum (apci) m/z=506.1 (M+H).

The following compounds were also prepared according to the method of Example 36, using the appropriate acid in Step C.

| Ex. # | Structure | Name | Data |
|---|---|---|---|
| 37 | 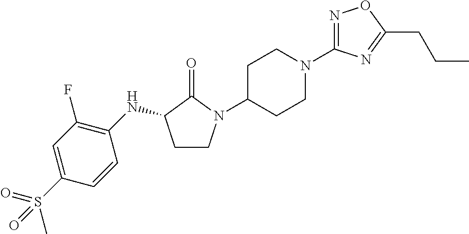 [SMILES: CCCc1nc(N2CCC(N3CC[C@H](Nc4ccc(S(C)(=O)=O)cc4F)C3=O)CC2)no1] | (S)-3-(2-fluoro-4- methylsulfonyl)phenylamino)- 1-(1-(5-propyl-1,2,4- oxadiazol-3-yl)piperidin-4- yl)pyrrolidin-2-one | Mass spectrum (apci) m/z = 466.1 (M + H) |
| 38 | 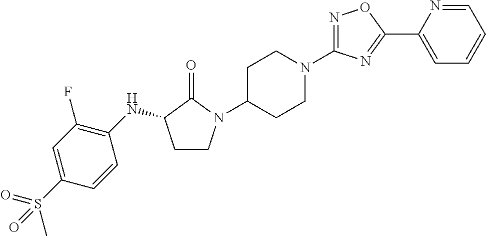 [SMILES: CS(=O)(=O)c1ccc(N[C@H]2CCN(C3CCN(c4noc(-c5ccccn5)n4)CC3)C2=O)c(F)c1] | (S)-3-(2-fluoro-4- (methylsulfonyl)- phenylamino)- 1-(1-(5-(pyridin-2-yl)- 1,2,4-oxadiazol-3- yl)piperidin-4-yl)pyrrolidin- 2-one | Mass spectrum (apci) m/z = 501.2 (M + H) |
| 39 | 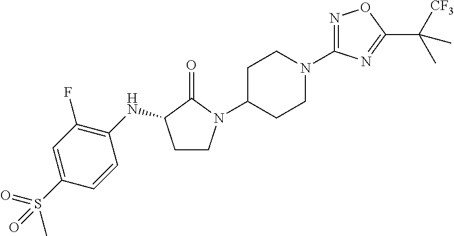 [SMILES: CC(C)(c1nc(N2CCC(N3CC[C@H](Nc4ccc(S(C)(=O)=O)cc4F)C3=O)CC2)no1)C(F)(F)F] | (S)-3-(2-fluoro-4- (methylsulfonyl)- phenylamino)- 1-(1-(5-(1,1,1-trifluoro-2- methylpropan-2-yl)-1,2,4- oxadiazol-3-yl)piperidin-4- yl)pyrrolidin-2-one | Mass spectrum (apci) m/z = 534.1 (M + H) |
| 40 | 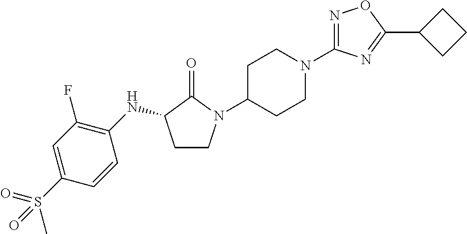 [SMILES: CS(=O)(=O)c1ccc(N[C@H]2CCN(C3CCN(c4noc(C5CCC5)n4)CC3)C2=O)c(F)c1] | (S)-1-(1-(5-cyclobutyl- 1,2,4-oxadiazol-3- yl)piperidin-4-yl)-3-(2- fluoro-4- (methylsulfonyl)- phenylamino)pyrrolidin- 2-one | Mass spectrum (apci) m/z = 478.1 (M + H) |
| 41 | 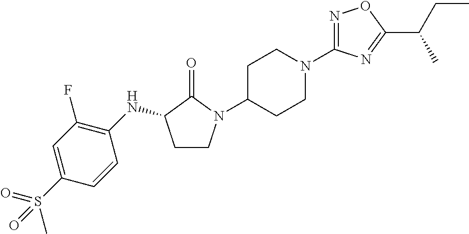 [SMILES: CC[C@H](C)c1nc(N2CCC(N3CC[C@H](Nc4ccc(S(C)(=O)=O)cc4F)C3=O)CC2)no1] | (S)-1-(1-(5-sec-butyl-1,2,4- oxadiazol-3-yl)piperidin-4- yl)-3-(2-fluoro-4- (methylsulfonyl)phenylamino)- pyrrolidin-2-one | Mass spectrum (apci) m/z = 480.1 (M + H) |
| 42 | 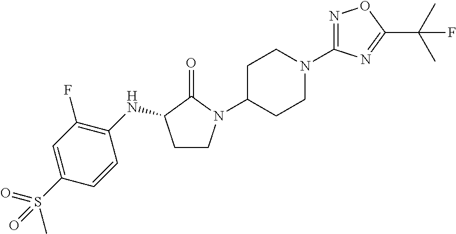 [SMILES: CC(C)(F)c1nc(N2CCC(N3CC[C@H](Nc4ccc(S(C)(=O)=O)cc4F)C3=O)CC2)no1] | (S)-3-(2-fluoro-4- (methylsulfonyl)- phenylamino)- 1-(1-(5-(2-fluoropropan- 2-yl)-1,2,4-oxadiazol-3- yl)piperidin-4-yl)pyrrolidin- 2-one | Mass spectrum (apci) m/z = 484.1 (M + H) |
| 43 | 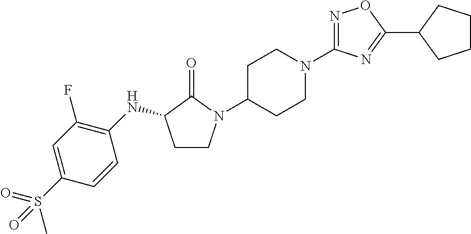 [SMILES: CS(=O)(=O)c1ccc(N[C@H]2CCN(C3CCN(c4noc(C5CCCC5)n4)CC3)C2=O)c(F)c1] | (S)-1-(1-(5-cyclopentyl- 1,2,4-oxadiazol-3- yl)piperidin-4-yl)-3-(2- fluoro-4- (methylsulfonyl)- phenylamino)- pyrrolidin-2-one | Mass spectrum (apci) m/z = 492.1 (M + H) |
| 44 | 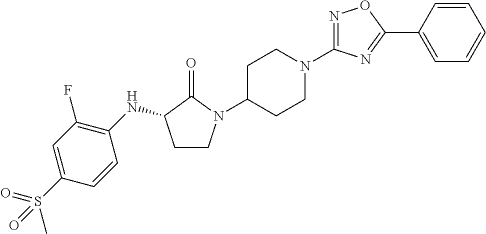 [SMILES: CS(=O)(=O)c1ccc(N[C@H]2CCN(C3CCN(c4noc(-c5ccccc5)n4)CC3)C2=O)c(F)c1] | (S)-3-(2-fluoro-4- (methylsulfonyl)- phenylamino)- 1-(1-(5-phenyl-1,2,4- oxadiazol-3-yl)piperidin-4- yl)pyrrolidin-2-one | Mass spectrum (apci) m/z = 500.2 (M + H) |
| 45 | 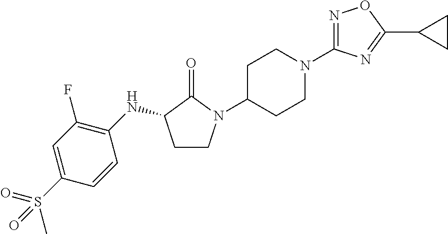 [SMILES: CS(=O)(=O)c1ccc(N[C@H]2CCN(C3CCN(c4noc(C5CC5)n4)CC3)C2=O)c(F)c1] | (S)-1-(1-(5-cyclopropyl- 1,2,4-oxadiazol-3- yl)piperidin-4-yl)-3-(2- fluoro-4- (methylsulfonyl)- phenylamino)- pyrrolidin-2-one | Mass spectrum (apci) m/z = 464.2 (M + H) |
| 46 | 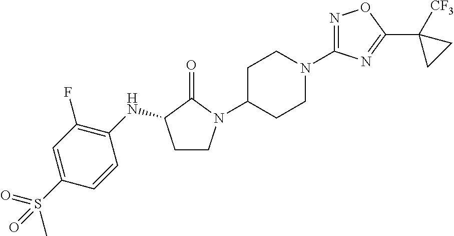 [SMILES: CS(=O)(=O)c1ccc(N[C@H]2CCN(C3CCN(c4noc(C5(C(F)(F)F)CC5)n4)CC3)C2=O)c(F)c1] | (S)-3-(2-fluoro-4- (methylsulfonyl)- phenylamino)- 1-(1-(5-(1- (trifluoromethyl)- cyclopropyl)- 1,2,4-oxadiazol-3- yl)piperidin-4- yl)pyrrolidin-2-one | Mass spectrum (apci) m/z = 532.1 (M + H) |
| 47 | 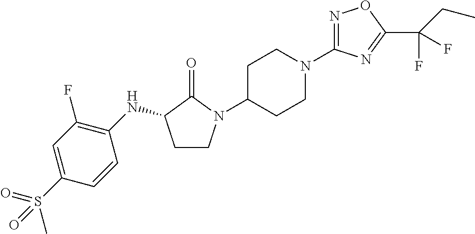 [SMILES: CCC(F)(F)c1nc(N2CCC(N3CC[C@H](Nc4ccc(S(C)(=O)=O)cc4F)C3=O)CC2)no1] | (S)-1-(1-(5-(1,1- difluoropropyl)-1,2,4- oxadiazol-3-yl)piperidin-4- yl)-3-(2-fluoro-4- (methylsulfonyl)- phenylamino)pyrrolidin- 2-one | ¹H NMR (400 MHz, CDCl₃): δ 1.22 (t, 3H), 1.74- 1.98 (m, 5H), 2.25-2.40 (m, 2H), 2.73-2.81 (m, 1H), 3.02 (s, 3H), 3.02-3.13 (m, 2H), 3.37- 3.50 (m, 2H), 4.10-4.22 (m, 3H), 4.22-4.32 (m, 1H), 5.07 (t, 1H), 6.79 (t, 1H), 7.55 (dd, 1H), 7.61 (d, 1H). |

### Example 48

### (S)-3-(2,5-difluoro-4-(methylsulfonyl)phenylamino)-1′-(5-(1,1-difluoroethyl)-1,2,4-oxadiazol-3-yl)-1,4′-bipiperidin-2-one

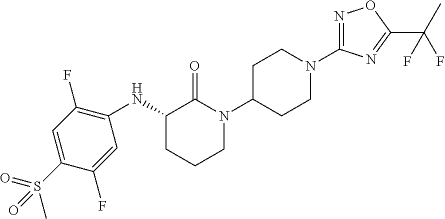 [SMILES: CC(F)(F)c1nc(N2CCC(N3CCC[C@H](Nc4cc(F)c(S(C)(=O)=O)cc4F)C3=O)CC2)no1]

Step A:

To a solution of (S)-3-(2,5-difluoro-4-(methylsulfonyl)phenylamino)-1,4′-bipiperidin-2-one (Preparation F-1; 1.1 g, 2.8 mmol) in acetonitrile (100 mL) was added potassium carbonate (0.82 g, 5.9 mmol) and cyanic bromide (5 M in acetonitrile, 0.68 mL, 3.4 mmol) and the reaction was stirred overnight at ambient temperature. The reaction was then diluted with 1 N NaOH solution and extracted with EtOAc. The organic layer was washed with 1 N NaOH, dried, and concentrated to give (S)-3-(2,5-difluoro-4-(methylsulfonyl)phenylamino)-2-oxo-1,4′-bipiperidine-1′-carbonitrile (0.98 g, 84%) as a white solid; MS (apci) m/z=413.2 (M+H).

Step B:

To a solution of (S)-3-(2,5-difluoro-4-(methylsulfonyl)phenylamino)-2-oxo-1,4′-bipiperidine-1′-carbonitrile (2.26 g, 5.4 mmol) in EtOH (60 mL) was added hydroxylamine (50% in water, 0.72 g, 10.9 mmol) and the reaction stirred at 60° C. for 2 hours. The solution was cooled and concentrated to give the crude (S)-3-(2,5-difluoro-4-(methylsulfonyl)phenylamino)-N-hydroxy-2-oxo-1,4′-bipiperidine-1′-carboximidamide (2.5 g, 100%) as an off-white solid; MS (apci) m/z=446.2 (M+H).

Step C:

To a solution of 2,2-difluoropropanoic acid (0.081 g, 0.74 mmol) in dioxane (6 mL) was added diisopropylethylamine (0.13 mL, 0.74 mmol) followed by addition of isobutyl carbonochloridate (0.10 mL, 0.74 mmol) at 0° C. After 1 hour, (S)-3-(2,5-difluoro-4-(methylsulfonyl)phenylamino)-N-hydroxy-2-oxo-1,4′-bipiperidine-1′-carboximidamide (0.220 g, 0.4938 mmol) was added and the reaction stirred at 60° C. overnight. The solution was cooled and diluted with water. The solution was extracted with EtOAc and the organic layer was dried over MgSO₄ and concentrated under vacuum. Reverse phase HPLC gave (S)-3-(2,5-difluoro-4-(methylsulfonyl)phenylamino)-1′-(5-(1,1-difluoroethyl)-1,2,4-oxadiazol-3-yl)-1,4′-bipiperidin-2-one (0.041 g, 16%) as a white solid. ¹H NMR (400 MHz, CDCl₃): δ 1.60-1.70 (m, 1H), 1.71-1.83 (m, 4H), 1.95-2.05 (m, 2H), 2.07 (t, 3H), 2.43-2.51 (m, 1H), 3.03-3.12 (m, 2H), 3.17 (s, 3H), 3.33 (t, 2H), 3.89-3.96 (m, 1H), 4.15-4.20 (m, 2H), 4.62-4.71 (m, 1H), 5.6 (bs, 1H), 6.47 (dd, 1H), 7.50 (dd, 1H).

### Example 49

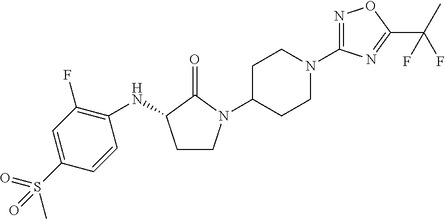 [SMILES: CC(F)(F)c1nc(N2CCC(N3CC[C@H](Nc4ccc(S(C)(=O)=O)cc4F)C3=O)CC2)no1]

### (S)-1-(1-(5-(1,1-difluoroethyl)-1,2,4-oxadiazol-3-yl)piperidin-4-yl)-3-(2-fluoro-4-(methylsulfonyl)phenylamino)pyrrolidin-2-one

Step A:

To a solution of cyanic bromide (0.14 g, 1.4 mmol) in acetonitrile (30 mL) was added potassium carbonate (0.31 g, 2.25 mmol) and (S)-3-(2-fluoro-4-(methylsulfonyl)phenylamino)-1-(piperidin-4-yl)pyrrolidin-2-one (Preparation C, 0.40 g, 1.1 mmol) and the reaction stirred for 90 minutes at room temperature. The reaction was then diluted with 1 N NaOH solution and extracted with EtOAc. The organic layer was washed with 1 N NaOH solution, dried over MgSO₄, and concentrated to give (S)-4-(3-(2-fluoro-4-(methylsulfonyl)phenylamino)-2-oxopyrrolidin-1-yl)piperidine-1-carbonitrile (0.32 g, 0.84 mmol, 75% yield) as a white solid. MS (apci) m/z=381.1 (M+H).

Step B:

To a solution of (S)-4-(3-(2-fluoro-4-(methylsulfonyl)phenylamino)-2-oxopyrrolidin-1-yl)piperidine-1-carbonitrile (0.92 g, 2.4 mmol) in EtOH (35 mL) was added hydroxylamine (0.32 g, 4.8 mmol) and the reaction stirred at 60° C. overnight. The solution was cooled and concentrated to give (S)-4-(3-(2-fluoro-4-(methylsulfonyl)phenylamino)-2-oxopyrrolidin-1-yl)-N-hydroxypiperidine-1-carboximidamide (0.98 g, 2.4 mmol, 98% yield) as a white solid. MS (apci) m/z=414.2 (M+H).

Step C:

2,2-Difluoropropanoic acid (0.28 g, 2.5 mmol) in dioxane (20 mL) was cooled to 0° C. Diisopropylethylamine (0.44 mL, 2.54 mmol) was added followed by isobutyl carbonochloridate (0.33 mL, 2.54 mmol). After 1 hour, (S)-4-(3-(2-fluoro-4-(methylsulfonyl)phenylamino)-2-oxopyrrolidin-1-yl)-N-hydroxypiperidine-1-carboximidamide (0.70 g, 1.69 mmol) was added and the reaction stirred at 60° C. overnight. The solution was cooled, diluted with water, extracted with EtOAc, dried over MgSO₄, and concentrated. Purification of the crude material by flash chromatography gave (S)-1-(1-(5-(1,1-difluoroethyl)-1,2,4-oxadiazol-3-yl)piperidin-4-yl)-3-(2-fluoro-4-(methylsulfonyl)phenylamino)pyrrolidin-2-one (0.075 g, 0.15 mmol, 9% yield) as a white solid. ¹H NMR (400 MHz, CDCl₃): δ 1.72-1.98 (m, 5H), 2.07 (t, 3H), 2.72-2.81 (m, 1H), 3.02 (s, 3H), 3.02-3.15 (m, 2H), 3.35-3.50 (m, 2H), 4.12-4.22 (m, 3H), 4.22-4.30 (m, 1H), 5.05-5.09 (m, 1H), 6.80 (t, 1H), 7.55 (d, 1H), 7.60 (d, 1H).

### Example 50

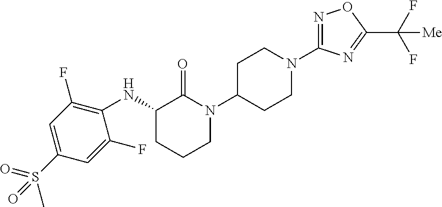 [SMILES: CC(F)(F)c1nc(N2CCC(N3CCC[C@H](Nc4c(F)cc(S(C)(=O)=O)cc4F)C3=O)CC2)no1]

### (S)-3-(2,6-difluoro-4-(methylsulfonyl)phenylamino)-1′-(5-(1,1-difluoroethyl)-1,2,4-oxadiazol-3-yl)-1,4′-bipiperidin-2-one

Prepared according to the method of Example 48 using (S)-3-(2,6-difluoro-4-(methylsulfonyl)phenylamino)-1,4′-bipiperidin-2-one (Preparation F-2) in Step A. Mass spectrum (apci) m/z=520.1 (M+H). ¹H NMR (400 MHz, CDCl₃): δ 1.58-1.69 (m, 1H), 1.70-1.82 (m, 4H), 1.90-2.00 (m, 2H), 2.05 (t, 3H), 2.42-2.51 (m, 1H), 3.02 (s, 3H), 3.00-3.10 (m, 2H), 3.28 (t, 2H), 4.12-4.20 (m, 2H), 4.30-4.38 (m, 1H), 4.60-4.70 (m, 1H), 5.12-5.20 (m, 1H), 7.42 (d, 2H).

### Example 51

### ((S)-3-(2-fluoro-4-(methylsulfonyl)phenylamino)-1-(1-(5-(pyrrolidin-1-yl)-1,2,4-oxadiazol-3-yl)piperidin-4-yl)pyrrolidin-2-one

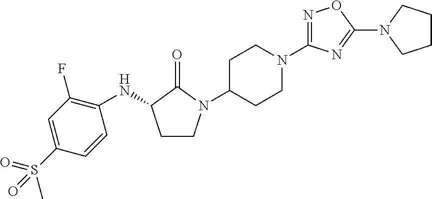 [SMILES: CS(=O)(=O)c1ccc(N[C@H]2CCN(C3CCN(c4noc(N5CCCC5)n4)CC3)C2=O)c(F)c1]

(S)-3-(2-Fluoro-4-(methylsulfonyl)phenylamino)-1-(1-(5-(trichloromethyl)-1,2,4-oxadiazol-3-yl)piperidin-4-yl)pyrrolidin-2-one (Example 13; 0.050 g, 0.092 mmol) and pyrrolidine (0.13 g, 1.9 mmol) were stirred in EtOH (1 mL) overnight. The material was concentrated under vacuum and purified by reverse phase HPLC to give (S)-3-(2-fluoro-4-(methylsulfonyl)phenylamino)-1-(1-(5-(pyrrolidin-1-yl)-1,2,4-oxadiazol-3-yl)piperidin-4-yl)pyrrolidin-2-one (0.022 g, 0.045 mmol, 48% yield) as a white solid. Mass spectrum (apci) m/z=493.2 (M+H).

### Example 52

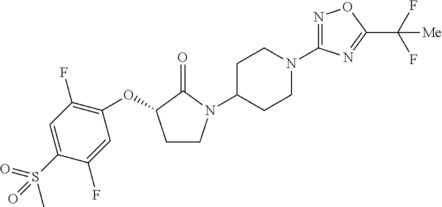 [SMILES: CC(F)(F)c1nc(N2CCC(N3CC[C@H](Oc4cc(F)c(S(C)(=O)=O)cc4F)C3=O)CC2)no1]

### (S)-3-(2,5-difluoro-4-(methylsulfonyl)phenoxy)-1-(1-(5-(1,1-difluoroethyl)-1,2,4-oxadiazol-3-yl)piperidin-4-yl)pyrrolidin-2-one

Prepared according to the method of Example 48 using (S)-3-(2,5-difluoro-4-(methylsulfonyl)phenoxy)-1-(piperidin-4-yl)pyrrolidin-2-one (Preparation F-4) in Step A. Mass spectrum (apci) m/z=507.0 (M+H). ¹H NMR (400 MHz, CDCl₃): δ 1.70-1.88 (m, 4H), 2.05 (t, 3H), 2.30-2.40 (m, 1H), 2.55-2.65 (m, 1H), 3.02-3.12 (m, 2H), 3.20 (s, 3H), 3.33-3.41 (m, 1H), 3.52-3.58 (m, 1H), 4.12-4.38 (m, 3H), 4.98 (t, 1H), 7.35 (dd, 1H), 7.67 (dd, 1H).

### Example 53

### (S)-3-(2,5-difluoro-4-(methylsulfonyl)phenylamino)-1-(1-(5-(trifluoromethyl)-1,2,4-oxadiazol-3-yl)piperidin-4-yl)azepan-2-one

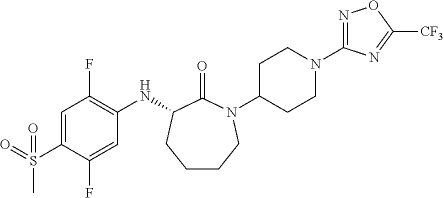 [SMILES: CS(=O)(=O)c1cc(F)c(N[C@H]2CCCCN(C3CCN(c4noc(C(F)(F)F)n4)CC3)C2=O)cc1F]

Step A:

(S)-3-(2,5-Difluoro-4-(methylsulfonyl)phenylamino)-1-(piperidin-4-yl)azepan-2-one was synthesized according to the method described in Preparation F, substituting (S)-6-amino-2-(benzyloxycarbonylamino)hexanoic acid for (S)-5-amino-2-(benzyloxycarbonylamino)pentanoic acid in Step A and 2,4-trifluoro-5-(methylsulfonyl)benzene for 1,2-difluoro-4-(methylsulfonyl)benzene in Step D.

Step B:

To a solution of (S)-3-(2,5-difluoro-4-(methylsulfonyl)phenylamino)-1-(piperidin-4-yl)azepan-2-one (0.60 g, 1.5 mmol) in acetonitrile (25 mL) was added potassium carbonate (0.43 g, 3.1 mmol) and cyanic bromide (5 M in acetonitrile, 0.36 mL, 1.8 mmol). The reaction was stirred overnight at ambient temperature. The reaction was made basic with 1 N NaOH solution. The solution was extracted with EtOAc (3×50 mL). The organic layer was dried over MgSO₄ and concentrated to give (S)-4-(3-(2,5-difluoro-4-(methylsulfonyl)phenylamino)-2-oxoazepan-1-yl)piperidine-1-carbonitrile (0.67 g, 100%) as a white solid.

Step C:

To a solution of (S)-4-(3-(2,5-difluoro-4-(methylsulfonyl)phenylamino)-2-oxoazepan-1-yl)piperidine-1-carbonitrile (0.67 g, 1.6 mmol) in EtOH (50 mL) was added hydroxylamine (0.21 g, 3.1 mmol, 50% in water) and the reaction was stirred in a sealed vessel at 60° C. overnight. The solution was cooled and concentrated under vacuum to give crude (S)-4-(3-(2,5-difluoro-4-(methylsulfonyl)phenylamino)-2-oxoazepan-1-yl)-N′-hydroxypiperidine-1-carboximidamide (0.60 g, 1.3 mmol, 83% yield) as a white solid.

Step D:

To a 0° C. solution of (S)-4-(3-(2,5-difluoro-4-(methylsulfonyl)phenylamino)-2-oxoazepan-1-yl)-N′-hydroxypiperidine-1-carboximidamide (0.60 g, 1.3 mmol) in dioxane (50 mL) was added trifluoroacetic anhydride (0.27 g, 1.3 mmol). After 30 minutes the mixture was heated to 60° C. and held at that temperature for 24 hours. The solution was cooled and concentrated under vacuum. The crude material was purified by column chromatography, eluting with 75% hexanes/ethyl acetate to provide (S)-3-(2,5-difluoro-4-(methylsulfonyl)phenylamino)-1-(1-(5-(trifluoromethyl)-1,2,4-oxadiazol-3-yl)piperidin-4-yl)azepan-2-one (0.21 g, 0.36 mmol, 28% yield) as a white solid. Mass spectrum (apci) m/z=535.9 (M−H).

### Example 54

### (S)-3-(2,5-difluoro-4-(methylsulfonyl)phenoxy)-1′-(5-(trifluoromethyl)-1,2,4-oxadiazol-3-yl)-[1,4′-bipiperidin]-2-one

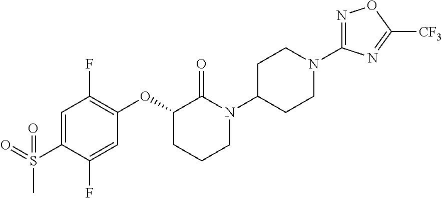 [SMILES: CS(=O)(=O)c1cc(F)c(O[C@H]2CCCN(C3CCN(c4noc(C(F)(F)F)n4)CC3)C2=O)cc1F]

Step A:

To a stirred solution of (methoxymethyl)triphenylphosphonium chloride (1.19 g, 3.48 mmol) in anhydrous ether (50 mL) at −10° C. under nitrogen was added phenyllithium (1.93 mL, 3.48 mmol) 1.8 M solution in diethyl ether, over 1 minute using a syringe. The mixture was stirred at 0° C. for 30 minutes and then cooled to −78° C. A solution of (R)-2-(2,2-dimethyl-5-oxo-1,3-dioxolan-4-yl)acetaldehyde (Preparation D, Step B; 0.500 g, 3.16 mmol) in ether/THF 1:1 (50 mL) was introduced via addition funnel, and the reaction mixture was stirred at −78° C. for 1 hour and then warmed to ambient temperature and stirred for 4 hours. The crude material was filtered and the residue was purified over silica gel (5-50% EtOAc in hexanes) to afford (R)-5-(3-methoxyallyl)-2,2-dimethyl-1,3-dioxolan-4-one (0.355 g, 1.90 mmol, 60% yield) as a clear, colorless oil (mixture of (E)- and (Z)-isomers).

Step B:

A solution of (R)-5-(3-methoxyallyl)-2,2-dimethyl-1,3-dioxolan-4-one (600 mg, 3.22 mmol) in acetone (32.2 mL, 3.22 mmol) and H₂SO₄ (1 drop) at ambient temperature was stirred for 70 minutes. Saturated aqueous NaHCO₃ (4-5 drops) was added and the mixture was concentrated in vacuo at ambient temperature. The residue was diluted with ether, washed with water, dried (Na₂SO₄), and concentrated in vacuo to afford (R)-3-(2,2-dimethyl-5-oxo-1,3-dioxolan-4-yl)propanal (407 mg, 0.938 mmol, 58% yield) as a yellow oil (2:1 mixture of aldehyde to dimethyl acetal).

Step C:

To a stirred solution of (R)-3-(2,2-dimethyl-5-oxo-1,3-dioxolan-4-yl)propanal (2.0 g, 8.71 mmol) in THF (120 mL) at 0° C. was added tert-butyl 4-aminopiperidine-1-carboxylate (1.92 g, 9.58 mmol). Sodium triacetoxyborohydride (2.77 g, 13.1 mmol) was added portionwise such that the internal temperature did not exceed 5° C. The mixture was stirred overnight while warming to ambient temperature. The reaction mixture was diluted with EtOAc and washed with brine. The aqueous layer was extracted twice with EtOAc, and the combined extracts were washed with brine, dried over magnesium sulfate and concentrated in vacuo. The residue was purified by reverse phase chromatography on C18 column (eluting with 0-60% ACN in water), to afford (R)-tert-butyl 3-hydroxy-2-oxo-[1,4′-bipiperidine]-1′-carboxylate as a light, white solid (1.55 g, 4.94 mmol, 57% yield). Mass spectrum (apci) m/z=199.1 (M+H-Boc).

Step D:

To a stirred solution of (R)-tert-butyl 3-hydroxy-2-oxo-1,4′-bipiperidine-1′-carboxylate (151 mg, 0.506 mmol) in THF (10 mL) at 8° C. was added N-ethyl-N-isopropylpropan-2-amine (0.176 μL, 1.01 mmol) in one portion. Methanesulfonyl chloride (47.3 μL, 0.607 mmol) was added at a rate such that the internal temperature did not exceed 5° C. After 45 minutes additional methanesulfonyl chloride (22 μL, 0.31 mmol) was added and stirring was continued for 15 minutes. To the reaction mixture was added 25 mL of EtOAc, followed by aqueous saturated NaHCO₃ (35 mL) via syringe at a rate such that the internal temperature did not exceed 5° C. The mixture was extracted with EtOAc, washed with, brine, dried (MgSO₄) and concentrated in vacuo. The residue was purified over silica gel (50-100% EtOAc in hexanes) to give (R)-tert-butyl 3-(methylsulfonyloxy)-2-oxo-1,4′-bipiperidine-1′-carboxylate (105 mg, 0.273 mmol, 54% yield). ¹H NMR (CDCl₃) δ 4.90 (m, 1H), 4.45 (m, 1H), 4.13 (m, 2H), 3.18 (s, 3H), 3.11 (m, 2H), 2.69 (m, 2H), 2.15 (m, 1H), 1.99 (m, 1H), 1.75 (m, 4H), 1.36 (s, 9H).

Step E:

To a stirred mixture of (R)-tert-butyl 3-(methylsulfonyloxy)-2-oxo-1,4′-bipiperidine-1′-carboxylate (1.10 g, 2.92 mmol) and potassium carbonate (485 mg, 3.51 mmol, 300 mesh, powdered) in THF (75 mL) was added 4-bromo-2,5-difluorophenol (733 mg, 3.51 mmol) and the reaction mixture was heated to reflux for 18 hours under nitrogen. The mixture was concentrated in vacuo and purified over silica gel (1:1 hexane/EtOAc) to afford (S)-tert-butyl 3-(4-bromo-2,5-difluorophenoxy)-2-oxo-1,4′-bipiperidine-1′-carboxylate obtained as a white solid (987 mg, 1.96 mmol, 67% yield). Mass spectrum (apci) m/z=389 (M+H-Boc). Chiral HPLC-analysis indicated this material was about 81% ee.

Normal Phase Chiral Method Conditions: Column: CHIRALPAK ADH (4.6×150 mm; 5 μm, Part #19324); UV: 222 nm; Sample preparation: 0.5 mg/mL methanol; Injection volume: 10 μL; Approximate retention times: (R)-enantiome: 9.2 minutes; (S)-enantiomer: 9.8 minutes.

### Gradient:

| | | Mobile phase A: | Mobile phase B: |
|---|---|---|---|
| | | hexanes | ethanol (200 proof) |
| Time (mins.) | Flow (mL/min) | (%) | (%) |
| 0 | 0.8 | 90 | 10 |
| 1 | 0.8 | 90 | 10 |
| 20 | 0.8 | 5 | 95 |
| 30 | 0.8 | 5 | 95 |

Step F:

A suspension of (S)-tert-butyl 3-(4-bromo-2,5-difluorophenoxy)-2-oxo-1,4′-bipiperidine-1′-carboxylate (700 mg, 1.39 mmol), and sodium methanesulfinate (219 mg, 2.08 mmol) in DMSO (5.55 mL), was deoxygenated and purged with nitrogen. Cu(I) triflate benzene complex (77.6 mg, 0.139 mmol) and (1S,2S)-cyclohexane-1,2-diamine (63.4 mg, 0.555 mmol) were introduced and the heterogeneous mixture was sealed and heated to 110° C. in an oil bath and stirred for 18 hours. The mixture was cooled to ambient temperature, diluted with EtOAc (75 mL), washed with water (30 mL) and brine (three 50 mL washes), dried (Na₂SO₄), filtered and concentrated in vacuo. The residue was purified over silica gel (EtOAc) to afford (S)-tert-butyl 3-(2,5-difluoro-4-(methylsulfonyl)phenoxy)-2-oxo-1,4′-bipiperidine-1′-carboxylate (305 mg, 0.606 mmol, 44% yield) as a light yellow oil that solidified. Mass spectrum (apci) m/z=389.1 (M+H-Boc).

Step G:

To a stirred solution of (S)-tert-butyl 3-(2,5-difluoro-4-(methylsulfonyl) phenoxy)-2-oxo-1,4′-bipiperidine-1′-carboxylate (370 mg, 0.757 mmol) in methanol (5 mL) was added, 5 M HCl in IPA (1.51 mL, 7.57 mmol) and the mixture was stirred at ambient temperature for 6 hours and concentrated in vacuo. The residue was stirred in 1M NaOH (20 mL) and DCM (25 mL). The organic layers were combined, dried (Na₂SO₄), filtered and concentrated in vacuo to give (S)-3-(2,5-difluoro-4-(methylsulfonyl)phenoxy)-[1,4′-bipiperidin]-2-one as light brown foam (282 mg, 0.726 mmol, 96% yield). Mass spectrum (apci) m/z=389.1 (M+H).

Step H:

(S)-3-(2,5-Difluoro-4-(methylsulfonyl)phenoxy)-1,4′-bipiperidin-2-one (0.99 g, 1.8 mmol) was dissolved in dry CH₃CN (5 mL) and treated with potassium carbonate (0.52 g, 3.7 mmol). Cyanic bromide (0.39 ml, 1.9 mmol, 5 M in CH₃CN) was added. The mixture was stirred for 30 minutes at ambient temperature. The reaction mixture was quickly poured into 1 N NaOH (10 mL) and extracted into EtOAc three times (10 mL each). The combined organics were dried (MgSO₄) and concentrated to give (S)-3-(2,5-difluoro-4-(methylsulfonyl)phenoxy)-2-oxo-1,4′-bipiperidine-1′-carbonitrile (0.81 g) as an off-white foam.

Step I:

Hydroxylamine (0.21 mL, 3.5 mmol, 50% in water) was added to a solution of (S)-3-(2,5-difluoro-4-(methylsulfonyl)phenoxy)-2-oxo-1,4′-bipiperidine-1′-carbonitrile (0.73 g, 1.7 mmol) in THF (4 mL). The solution was stirred at ambient temperature for 2 hours and then concentrated to afford (S,E)-3-(2,5-difluoro-4-(methylsulfonyl)phenoxy)-N′-hydroxy-2-oxo-1,4′-bipiperidine-1′-carboximidamide (0.90 g).

Step J:

A solution of (S,E)-3-(2,5-difluoro-4-(methylsulfonyl)phenoxy)-N′-hydroxy-2-oxo-1,4′-bipiperidine-1′-carboximidamide (0.75 g, 1.67 mmol) in THF (40 mL) was placed in an ice bath and 2,2,2-trifluoroacetic anhydride (0.35 mL, 2.5 mmol) was added. The reaction was allowed to warm to ambient temperature with overnight stirring. Ethyl acetate (40 mL) was added followed by addition of a saturated Na₂CO₃ solution (10 mL). The organic layer was separated and washed with brine and concentrated. Flash chromatography gave (S)-3-(2,5-difluoro-4-(methylsulfonyl)phenoxy)-1′-(5-(trifluoromethyl)-1,2,4-oxadiazol-3-yl)-1,4′-bipiperidin-2-one (0.68 g, 77%) as a white solid. ¹H NMR (400 MHz, CDCl₃): δ 7.64 (m, 1H), 7.23 (m, 1H), 4.81 (m, 1H), 4.79 (m, 1H), 4.18 (m, 2H), 3.31 (m, 2H), 3.18 (s, 3H), 3.08 (m, 2H), 2.19 (m, 3H), 1.91 (m, 5H). Chiral HPLC: 80% ee.

### Example 55

### (S)-3-(2,5-difluoro-4-(methylsulfonyl)phenoxy)-1′-(5-(1,1-difluoroethyl)-1,2,4-oxadiazol-3-yl)-[1,4′-bipiperidin]-2-one

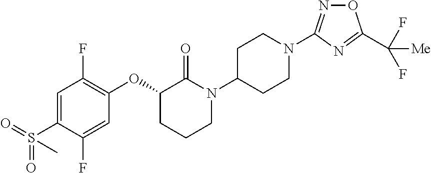 [SMILES: CC(F)(F)c1nc(N2CCC(N3CCC[C@H](Oc4cc(F)c(S(C)(=O)=O)cc4F)C3=O)CC2)no1]

(S,E)-3-(2,5-Difluoro-4-(methylsulfonyl)phenoxy)-N′-hydroxy-2-oxo-1,4′-bipiperidine-1′-carboximidamide (Prepared in example 54, Step I) (0.090 g, 0.20 mmol) was charged with DCM (3 mL) and triethylamine (0.042 mL, 0.30 mmol) under a nitrogen atmosphere. 2,2-Difluoroacetyl chloride (0.28 mL, 0.22 mmol, 0.8 M solution in DCE) was added in about 50 μL portions over 5 minutes. The mixture was stirred at 60° C. for 1 day. The reaction mixture was concentrated and purified by reverse phase chromatography to provide (S)-3-(2,5-difluoro-4-(methylsulfonyl)phenoxy)-1′-(5-(1,1-difluoroethyl)-1,2,4-oxadiazol-3-yl)-[1,4′-bipiperidin]-2-one (0.015 g, 14%). Mass spectrum (apci) m/z=495.1.

### Example 56

### (S)-3-((6-(methylsulfonyl)pyridin-3-yl)oxy)-1-(1-(5-(trifluoromethyl)-1,2,4-oxadiazol-3-yl)piperidin-4-yl)pyrrolidin-2-one

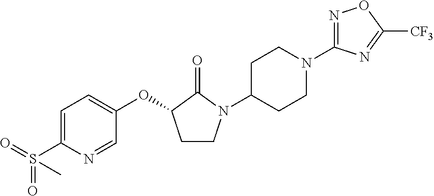 [SMILES: CS(=O)(=O)c1ccc(O[C@H]2CCN(C3CCN(c4noc(C(F)(F)F)n4)CC3)C2=O)cn1]

Prepared according to the method of Example 12 using (S)-3-((6-(methylsulfonyl)pyridin-3-yl)oxy)-1-(piperidin-4-yl)pyrrolidin-2-one (Preparation G) in Step A and using 2,2,2-trifluoroacetic anhydride in Step C. Mass spectrum (apci) m/z=476.0.

### Example 57

### (S)-3-((6-(methylsulfonyl)pyridin-3-yl)amino)-1-(1-(5-(trifluoromethyl)-1,2,4-oxadiazol-3-yl)piperidin-4-yl)pyrrolidin-2-one

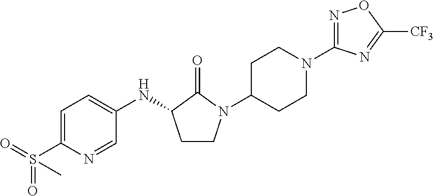 [SMILES: CS(=O)(=O)c1ccc(N[C@H]2CCN(C3CCN(c4noc(C(F)(F)F)n4)CC3)C2=O)cn1]

Prepared according to the method of Example 12 using (S)-3-(6-(methylsulfonyl)pyridin-3-ylamino)-1-(piperidin-4-yl)pyrrolidin-2-one (Preparation H) in Step A and using 2,2,2-trifluoroacetic anhydride in Step C. Mass spectrum (apci) m/z=475.0.

### Example 58

### (S)-1-(1-(5-difluoromethyl)-1,2,4-oxadiazol-3-yl)piperidin-4-yl)-3-((6-(methylsulfonyl)pyridin-3-yl)amino)pyrrolidin-2-one

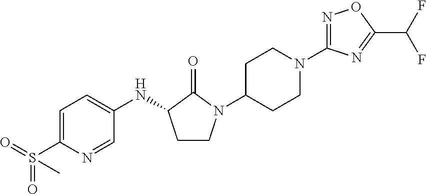 [SMILES: CS(=O)(=O)c1ccc(N[C@H]2CCN(C3CCN(c4noc(C(F)F)n4)CC3)C2=O)cn1]

Prepared according to the method of Example 12 using (S)-3-(6-(methylsulfonyl)pyridin-3-ylamino)-1-(piperidin-4-yl)pyrrolidin-2-one (Preparation H) in Step A and using 2,2-difluoroacetic anhydride in Step C. Mass spectrum (apci) m/z=457.0.

### Example 59

### (S)-3-((2,5-difluoro-4-(methylsulfonyl)phenyl)amino)-1-(1-(3-(trifluoromethyl)-1,2,4-oxadiazol-5-yl)piperidin-4-yl)pyrrolidin-2-one

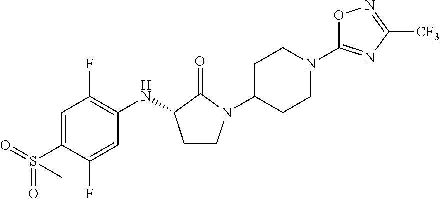 [SMILES: CS(=O)(=O)c1cc(F)c(N[C@H]2CCN(C3CCN(c4nc(C(F)(F)F)no4)CC3)C2=O)cc1F]

Prepared according to the method of Example 1 using (S)-3-(2,5-difluoro-4-(methylsulfonyl)phenylamino)-1-(piperidine-4-yl)pyrrolidin-2-one (Preparation C-1) in Step A and using 2,2,2-trifluoro-N-hydroxyacetimidamide in Step B. Mass spectrum (apci) m/z=508.0 (M−H).

### Example 60

### (S)-3-((2,6-difluoro-4-(methylsulfonyl)phenyl)amino)-1-(1-(3-(trifluoromethyl)-1,2,4-oxadiazol-5-yl)piperidin-4-yl)pyrrolidin-2-one

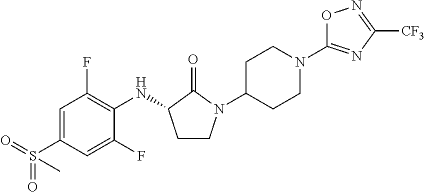 [SMILES: CS(=O)(=O)c1cc(F)c(N[C@H]2CCN(C3CCN(c4nc(C(F)(F)F)no4)CC3)C2=O)c(F)c1]

Prepared according to the method of Example 1 using (S)-3-(2,6-difluoro-4-(methylsulfonyl)phenylamino)-1-(piperidin-4-yl)pyrrolidin-2-one (Preparation C-2) in Step A and using 2,2,2-trifluoro-N-hydroxyacetimidamide in Step B. Mass spectrum (apci) m/z=507.9 (M−H).

### Example 61

### (S)-3-((2,5-difluoro-4-(methylsulfonyl)phenyl)amino)-1′-(3-(1,1-difluoroethyl)-1,2,4-oxadiazol-5-yl)-[1,4′-bipiperidin]-2-one

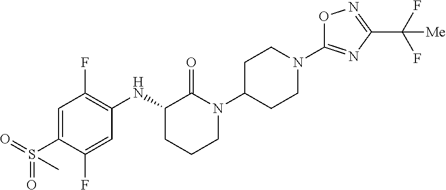 [SMILES: CC(F)(F)c1noc(N2CCC(N3CCC[C@H](Nc4cc(F)c(S(C)(=O)=O)cc4F)C3=O)CC2)n1]

Prepared according to the method of Example 1 using (S)-3-(2,5-difluoro-4-(methylsulfonyl)phenylamino)-1,4′-bipiperidin-2-one (Preparation F-1) in Step A and using 2,2-difluoro-N-hydroxypropanimidamide in Step B. Mass spectrum (apci) m/z=520.0 (M+H).

### Example 62

### (S)-3-((2,6-difluoro-4-(methylsulfonyl)phenyl)amino)-1′-(3-(1,1-difluoroethyl)-1,2,4-oxadiazol-5-yl)-[1,4′-bipiperidin]-2-one

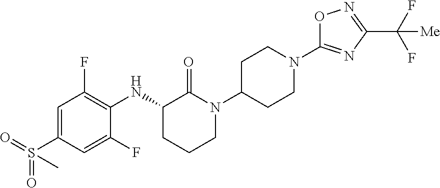 [SMILES: CC(F)(F)c1noc(N2CCC(N3CCC[C@H](Nc4c(F)cc(S(C)(=O)=O)cc4F)C3=O)CC2)n1]

Prepared according to the method of Example 1 using (S)-3-(2,6-difluoro-4-(methylsulfonyl)phenylamino)-1,4′-bipiperidin-2-one in (Preparation F-2) in Step A and using 2,2-difluoro-N-hydroxypropanimidamide in Step B. Mass spectrum (apci) m/z=518.0 (M−H).

### Example 63

### (S)-3-(2,5-difluoro-4-(methylsulfonyl)phenoxy)-1-(1-(3-(1,1-difluoroethyl)-1,2,4-oxadiazol-5-yl)piperidin-4-yl)pyrrolidin-2-one

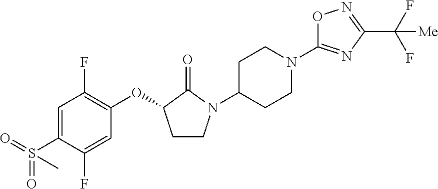 [SMILES: CC(F)(F)c1noc(N2CCC(N3CC[C@H](Oc4cc(F)c(S(C)(=O)=O)cc4F)C3=O)CC2)n1]

Prepared according to the method of Example 1 using (S)-3-(2,5-difluoro-4-(methylsulfonyl)phenoxy)-1-(piperidin-4-yl)pyrrolidin-2-one (Preparation F-4) in Step A and using 2,2-difluoro-N-hydroxypropanimidamide in Step B. Mass spectrum (apci) m/z=507.0 (M+H).

### Example 64

### (S)-3-((2,5-difluoro-4-(methylsulfonyl)phenyl)amino)-1-(1-(5-(1,1-difluoroethyl)-1,2,4-oxadiazol-3-yl)piperidin-4-yl)pyrrolidin-2-one

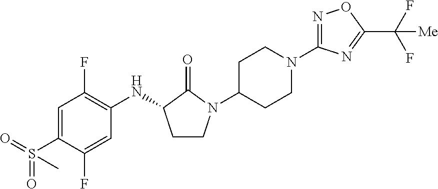 [SMILES: CC(F)(F)c1nc(N2CCC(N3CC[C@H](Nc4cc(F)c(S(C)(=O)=O)cc4F)C3=O)CC2)no1]

Prepared according to the method of Example 48 using (S)-3-(2,5-difluoro-4-(methylsulfonyl)phenylamino)-1-(piperidin-4-yl)pyrrolidin-2-one (Preparation C-1) in Step A. Mass spectrum (apci) m/z=506.0 (M+H).

### Example 65

### (S)-3-((2,6-difluoro-4-(methylsulfonyl)phenyl)amino)-1-(1-(5-(1,1-difluoroethyl)-1,2,4-oxadiazol-3-yl)piperidin-4-yl)pyrrolidin-2-one

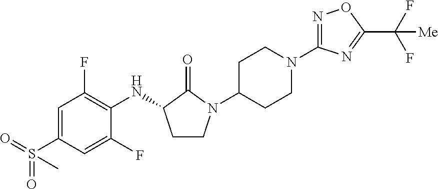 [SMILES: CC(F)(F)c1nc(N2CCC(N3CC[C@H](Nc4c(F)cc(S(C)(=O)=O)cc4F)C3=O)CC2)no1]

Prepared according to the method of Example 48 using (S)-3-(2,6-difluoro-4-(methylsulfonyl)phenylamino)-1-(piperidin-4-yl)pyrrolidin-2-one (Preparation C-2) in Step A. Mass spectrum (apci) m/z=506.0 (M+H).

### Example 66

### (R)-3-(2,5-difluoro-4-(methylsulfonyl)phenoxy)-1-(1-(5-(trifluoromethyl)-1,2,4-oxadiazol-3-yl)piperidin-4-yl)pyrrolidin-2-one

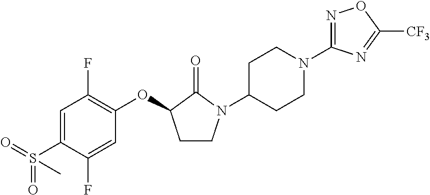 [SMILES: CS(=O)(=O)c1cc(F)c(O[C@@H]2CCN(C3CCN(c4noc(C(F)(F)F)n4)CC3)C2=O)cc1F]

Step A:

tert-Butyl 4-aminopiperidine-1-carboxylate (10 g, 50.0 mmol) was suspended in DCM. Triethylamine (7.6 g, 75 mmol) was added and the reaction mixture was cooled in an ice bath. 2,4-Dibromobutanoyl chloride (13.2 g 50 mmol) was added over one minute and allowed to stir at 0° C. After 4 hours, the reaction was poured into a saturated NaHCO₃ solution, and the mixture was extracted with DCM. The organic layer was dried, filtered and concentrated to afford tert-butyl 4-(2,4-dibromobutanamido)piperidine-1-carboxylate (22 g) which was used without any further purification

Step B:

NaH (2.0 g, 49 mmol, 60% dispersion in mineral oil) was added to a 0° C. solution of tert-butyl 4-(2,4-dibromobutanamido)piperidine-1-carboxylate (21 g, 49 mmol) in DMF (50 mL). The reaction was allowed to warm to ambient temperature and stirred for 4 hours. The reaction was poured into brine (500 mL) and extracted with ethyl acetate (3×200 mL). The combined organic layers were washed with brine (3×200 mL), dried over MgSO₄ and concentrated in vacuo. Purification of the crude material by flash chromatography (50% to 100% EtOAc/hexanes) gave tert-butyl 4-(3-bromo-2-oxopyrrolidin-1-yl)piperidine-1-carboxylate (9 g, 53%).

Step C:

To a solution of potassium carbonate (4.78 g, 34.6 mmol) in acetone was added 4-bromo-2,5-difluorophenol (4.87 g, 23.3 mmol) and the reaction stirred for 10 minutes. tert-Butyl 4-(3-bromo-2-oxopyrrolidin-1-yl)piperidine-1-carboxylate (6 g, 17.3 mmol) was added and the reaction stirred at ambient temperature overnight. The reaction was concentrated in vacuo and the residue was partitioned between EtOAc and 1N NaOH. The combined organic layers were separated, washed with water, brine, dried over MgSO4 and concentrated in vacuo. The material was purified by flash chromatography (20% EtOAc/DCM) to obtain tert-butyl 4-(3-(4-bromo-2,5-difluorophenoxy)-2-oxopyrrolidin-1-yl)piperidine-1-carboxylate (5.7 g, 69%).

Step D:

tert-Butyl 4-(3-(4-bromo-2,5-difluorophenoxy)-2-oxopyrrolidin-1-yl)piperidine-1-carboxylate (5.0 g, 10.5 mmol), sodium methanesulfinate (1.61 g, 15.8 mmol) and (1S,2S)-cyclohexane-1,2-diamine (0.48 g, 4.21 mmol) were dissolved in DMSO (100 mL). The mixture was bubbled through with nitrogen for 5 minutes. (CuOTf)₂Ph complex (0.59 g, 1.05 mmol) was added and the reaction stirred at 110° C. under nitrogen overnight. The reaction was cooled to ambient temperature, poured into water (1 L) and extracted with EtOAc (3×250 mL). The combined organic layers were washed with brine, dried over MgSO4 and concentrated in vacuo. The residue was purified by flash chromatography (50% to 100% EtOAc/hexanes) to afford tert-butyl 4-(3-(2,5-difluoro-4-(methylsulfonyl)phenoxy)-2-oxopyrrolidin-1-yl)piperidine-1-carboxylate (2.4 g, 48% yield).

Step E:

To a 0° C. solution of tert-butyl 4-(3-(2,5-difluoro-4-(methylsulfonyl)phenoxy)-2-oxopyrrolidin-1-yl)piperidine-1-carboxylate (2.4 g, 5.1 mmol) in ethyl acetate (100 mL) was added HCl (10 ml, 51 mmol) in isopropanol. The reaction was stirred at ambient temperature overnight. The solution was concentrated and neutralized using 1 N NaOH solution. The material was extracted into DCM (3×100 mL). The combined organic layers were washed with brine, dried over MgSO₄, and concentrated to give 3-(2,5-difluoro-4-(methylsulfonyl)phenoxy)-1-(piperidin-4-yl)pyrrolidin-2-one (1.8 g, 95%).

Step F:

To a solution of cyanogen bromide (1.2 ml, 5.8 mmol) in MeCN (125 mL) was added potassium carbonate (1.4 g, 10 mmol) and 3-(2,5-difluoro-4-(methylsulfonyl)phenoxy)-1-(piperidin-4-yl)pyrrolidin-2-one (1.8 g, 4.8 mmol). This mixture stirred at ambient temperature overnight. The reaction was poured into 1N NaOH (100 mL) and extracted into ethyl acetate (3×50 mL). The combined organic layers were dried over MgSO₄ and concentrated in vacuo to give 4-(3-(2,5-difluoro-4-(methylsulfonyl)phenoxy)-2-oxopyrrolidin-1-yl)piperidine-1-carbonitrile (1.9 g, 99%).

Step G:

To a solution of 4-(3-(2,5-difluoro-4-(methylsulfonyl)phenoxy)-2-oxopyrrolidin-1-yl)piperidine-1-carbonitrile (1.9 g, 4.8 mmol) in EtOH (50 mL) was added hydroxylamine (0.63 g, 9.5 mmol) and the reaction stirred at 60° C. overnight. The solution was concentrated in vacuo to give the crude (R,E)-4-(3-(2,5-difluoro-4-(methylsulfonyl)phenoxy)-2-oxopyrrolidin-1-yl)-N′-hydroxypiperidine-1-carboximidamide (2.1 g) which was used in the next step without further purification.

Step H:

A solution of (R,E)-4-(3-(2,5-difluoro-4-(methylsulfonyl)phenoxy)-2-oxopyrrolidin-1-yl)-N′-hydroxypiperidine-1-carboximidamide (0.25 g, 0.58 mmol) and trifluoroacetic anhydride (0.24 g, 1.2 mmol) in dioxane (10 mL) was heated to 60° C. in a sealed tube for 4 hours. The solution was concentrated in vacuo and the product was purified by column chromatography (25% to 75% EtOAc/hexanes) to afford the racemic product. A portion of this material was separated using a 21×250 mm, Chiralcel OJH, PN 17345 column eluting with 60/20/20 mixture of hexanes/ethanol/methanol with a flow rate of 21 mL/minute (Lamda max at 237 nm, peak 2) to afford (R)-3-(2,5-difluoro-4-(methylsulfonyl)phenoxy)-1-(1-(5-(trifluoromethyl)-1,2,4-oxadiazol-3-yl)piperidin-4-yl)pyrrolidin-2-one. Mass spectrum (apci) m/z=506.0 (M+H). ¹H NMR (400 MHz, CDCl₃): δ 1.72-1.90 (m, 4H), 2.30-2.40 (m, 1H), 2.57-2.65 (m, 1H), 3.08-3.15 (m, 2H), 3.20 (s, 3H), 3.35-3.42 (m, 1H), 3.51-3.58 (m, 1H), 4.13-4.27 (m, 3H), 4.98 (t, 1H), 7.35 (dd, 1H), 7.67 (dd, 1H).

### Example 67

### (R)-3-(2,5-difluoro-4-(methylsulfonyl)phenoxy)-1′-(5-(trifluoromethyl)-1,2,4-oxadiazol-3-yl)-[1,4′-bipiperidin]-2-one

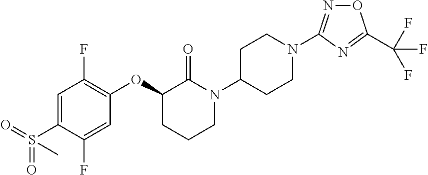 [SMILES: CS(=O)(=O)c1cc(F)c(O[C@@H]2CCCN(C3CCN(c4noc(C(F)(F)F)n4)CC3)C2=O)cc1F]

Prepared according the method of Example 54. The (R) enantiomer was isolated from the enriched (S)-3-(2,5-difluoro-4-(methylsulfonyl)phenoxy)-1′-(5-(trifluoromethyl)-1,2,4-oxadiazol-3-yl)-[1,4′-bipiperidin]-2-one product mixture from Example 54 using optimized preparative SFC method parameters: Column. AS 20 mm×250 mm, Flow Rate: 60 mL/min, MPA: 85% Supercritical CO₂, MPB: 15% Methanol. ¹H NMR (400 MHz, CDCl₃): δ 7.64 (m, 1H), 7.23 (m, 1H), 4.81 (m, 1H), 4.79 (m, 1H), 4.18 (m, 2H), 3.31 (m, 2H), 3.18 (s, 3H), 3.08 (m, 2H), 2.19 (m, 3H), 1.91 (m, 5H). Enantiopurity was assessed at 93% ee.

1. A compound having the general formula I 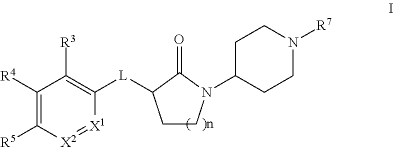
or a pharmaceutically acceptable salt thereof, wherein:
L is O or NR^{x};
R^{x} is H or (1-3C)alkyl;
X¹ is N or CR¹ and X² is N or CR², wherein only one of X¹ and X² may be N;
R¹, R², R³ and R⁴ are independently selected from H, halogen, CF₃, (1-6C)alkyl and (1-6C)alkoxy;
R⁵ is (1-3C alkyl)sulfonyl, (3-6C cycloalkyl)sulfonyl, (cyclopropylmethyl)sulfonyl, phenylsulfonyl, CN, Br, CF₃, or tetrazolyl optionally substituted with (1-3C)alkyl;
R⁷ is selected from 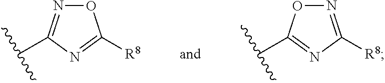
R⁸ is (1-6C)alkyl, fluoro(1-6C)alkyl, difluoro(1-6C)alkyl, trifluoro(1-6C)alkyl, trichloro(1-6C)alkyl, Cyc¹, Ar¹, hetCyc¹ or hetAr¹;
Cyc¹ is (3-6C)cycloalkyl optionally substituted with CF₃;
Ar¹ is phenyl optionally substituted with one or more groups independently selected from halogen, CF₃, (1-4C)alkyl and (1-4C)alkoxy;
hetCyc¹ is a 5-6 membered heterocycle having a ring nitrogen atom and optionally substituted with one or more groups independently selected from halogen, CF₃, (1-4C)alkyl and (1-4C)alkoxy;
hetAr¹ is a 6-membered heteroaryl having a ring nitrogen atom and optionally substituted with one or more groups independently selected from halogen, CF₃, (1-4C)alkyl and (1-4C)alkoxy; and
n is 1, 2 or 3.

2. The compound according to claim 1, wherein:
X¹ is CR¹;
X² is CR²; and
R¹, R², R³ and R⁴ are independently selected from H, (1-6C)alkyl, CF₃ and halogen.

3. The compound of claim 2, wherein:
R¹ and R² are independently selected from H, F and Cl; and
R³ and R⁴ are independently selected from H, Me, F, Cl and CF₃.

4. The compound of claim 3, wherein:
R¹ and R³ are F; and
R² and R⁴ are H.

5. The compound of claim 3, wherein:
R¹ and R⁴ are H; and
R² and R³ are F.

6. The compound of claim 3, wherein:
R¹, R² and R⁴ are H; and
R³ is F.

7. The compound according to claim 1, wherein:
X¹ is N; and
X² is CR².

8. The compound of claim 7, wherein R², R³ and R⁴ are independently selected from H, halogen, and (1-6C)alkyl.

9. The compound of claim 8, wherein R², R³ and R⁴ are each H.

10. The compound of claim 8, wherein R² and R⁴ are H and R³ is Cl or F.

11. The compound according to claim 1, wherein:
X¹ is CR¹; and
X² is N.

12. The compound according to claim 11, wherein R¹, R³ and R⁴ are independently selected from H, halogen, and (1-6C)alkyl.

13. The compound according to claim 12, wherein each of R¹, R³ and R⁴ is H.

14. The compound according to claim 12, wherein R¹ and R⁴ are H and R³ is Cl or F.

15. The compound according to claim 1, wherein R⁵ is selected from (1-3C alkyl)sulfonyl, (3-6C cycloalkyl)sulfonyl-, (cyclopropylmethyl)sulfonyl, and phenylsulfonyl (C₆H₅SO₂—).

16. The compound according to claim 15, wherein R⁵ is (1-3C alkyl)sulfonyl.

17. The compound according to claim 16, wherein R⁵ is methylsulfonyl.

18. The compound according to claim 1, wherein R⁵ is selected from CN, Br and CF₃.

19. The compound according to claim 1, wherein R⁵ is tetrazolyl optionally substituted with (1-3C)alkyl.

20. The compound according to claim 1, wherein R⁷ is

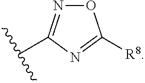

21. The compound of claim 20, wherein R⁸ is selected from (1-6C)alkyl, fluoro(1-6C)alkyl, difluoro(1-6C)alkyl, trifluoro(1-6C)alkyl and trichloro(1-6C)alkyl.

22. The compound of claim 21, wherein R⁸ is selected from ethyl, isopropyl, propyl, sec-propyl, tert-butyl, 2-fluoropropyl, difluoromethyl, 1,1-difluoroethyl, 1,1-difluoropropyl, trifluoromethyl and 1,1-dimethyl-2,2-difluoroethyl.

23. The compound of claim 22, wherein R⁸ is selected from 2-fluoropropyl, difluoromethyl, 1,1-difluoroethyl, 1,1-difluoropropyl, trifluoromethyl and 1,1-dimethyl-2,2-difluoroethyl.

24. The compound of claim 20, wherein R⁸ is selected from Cyc¹, Ar¹, hetCyc¹ and hetAr¹.

25. The compound of claim 24 wherein R⁸ is selected from cyclopropyl, 1-(trifluoromethyl)cyclopropyl, cyclobutyl, cyclopentyl, phenyl, pyrrolidin-1-yl and pyrid-2-yl.

26. The compound according to claim 1, wherein R⁷ is

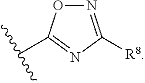

27. The compound of claim 26, wherein R⁸ is selected from (1-6C)alkyl, fluoro(1-6C)alkyl, difluoro(1-6C)alkyl, trifluoro(1-6C)alkyl and trichloro(1-6C)alkyl.

28. The compound of claim 27, wherein R⁸ is selected from ethyl, isopropyl, propyl, sec-propyl, tert-butyl, 2-fluoropropyl, difluoromethyl, 1,1-difluoroethyl, 1,1-difluoropropyl, trifluoromethyl and 1,1-dimethyl-2,2-difluoroethyl.

29. The compound of claim 28, wherein R⁸ is selected from 2-fluoropropyl, difluoromethyl, 1,1-difluoroethyl, 1,1-difluoropropyl, trifluoromethyl and 1,1-dimethyl-2,2-difluoroethyl.

30. The compound of claim 26, wherein R⁸ is selected from Cyc¹, Ar¹, hetCyc¹ and hetAr¹.

31. The compound of claim 30 wherein R⁸ is selected from cyclopropyl, 1-(trifluoromethyl)cyclopropyl, cyclobutyl, cyclopentyl, phenyl, pyrrolidin-1-yl and pyrid-2-yl.

32. The compound according to claim 1, wherein L is O.

33. The compound according to claim 1, wherein L is NR^{x}.

34. The compound according to claim 33, wherein L is NH.

35. The compound according to claim 1, wherein n is 1.

36. The compound according to claim 1, wherein n is 2.

37. The compound according to claim 1, wherein n is 3.

38. The compound according to claim 1, having the absolute configuration of Formula I-a:

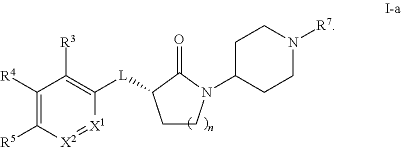

39. The compound according to claim 1, having the absolute configuration of Formula I-b:

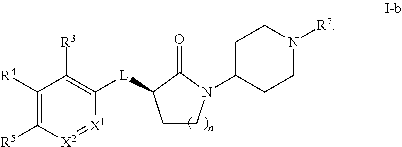

40. A compound of Formula I as defined in claim 1 selected from
(S)-3-(2,5-difluoro-4-(methylsulfonyl)phenoxy)-1-(1-(3-isopropyl-1,2,4-oxadiazol-5-yl)piperidin-4-yl)pyrrolidin-2-one;
(S)-3-(2-fluoro-4-(methylsulfonyl)phenoxy)-1-(1-(3-isopropyl-1,2,4-oxadiazol-5-yl)piperidin-4-yl)pyrrolidin-2-one;
(S)-1-(1-(3-tert-butyl-1,2,4-oxadiazol-5-yl)piperidin-4-yl)-3-(2,5-difluoro-4-(methylsulfonyl)phenoxy)pyrrolidin-2-one;
(R)-3-(2,5-difluoro-4-(methylsulfonyl)phenoxy)-1-(1-(3-isopropyl-1,2,4-oxadiazol-5-yl)piperidin-4-yl)pyrrolidin-2-one;
(S)-1-(1-(3-tert-butyl-1,2,4-oxadiazol-5-yl)piperidin-4-yl)-3-(2-fluoro-4-(methylsulfonyl)phenoxy)pyrrolidin-2-one;
(S)-1-(1-(3-ethyl-1,2,4-oxadiazol-5-yl)piperidin-4-yl)-3-(2-fluoro-4-(methylsulfonyl)phenoxy)pyrrolidin-2-one;
(S)-3-(2-fluoro-4-(methylsulfonyl)phenylamino)-1′-(3-isopropyl-1,2,4-oxadiazol-5-yl)-1,4′-bipiperidin-2-one;
(S)-3-(2-fluoro-4-(methylsulfonyl)phenylamino)-1-(1-(3-isopropyl-1,2,4-oxadiazol-5-yl)piperidin-4-yl)pyrrolidin-2-one;
(S)-3-(2,5-difluoro-4-(methylsulfonyl)phenylamino)-1′-(3-trifluoromethyl-1,2,4-oxadiazol-5-yl)-1,4′-bipiperidin-2-one;
(S)-3-(2,6-difluoro-4-(methylsulfonyl)phenylamino)-1′-(3-trifluoromethyl-1,2,4-oxadiazol-5-yl)-1,4′-bipiperidin-2-one;
(S)-1-(1-(3-trifluoromethyl-1,2,4-oxadiazol-5-yl)piperidin-4-yl)-3-(2,5-difluoro-4-(methylsulfonyl)phenoxy)pyrrolidin-2-one;
(S)-3-(2,6-difluoro-4-(methylsulfonyl)phenylamino)-1′-(5-isopropyl-1,2,4-oxadiazol-3-yl)-1,4′-bipiperidin-2-one;
(S)-3-(2-fluoro-4-(methylsulfonyl)phenylamino)-1-(1-(5-(trichloromethyl)-1,2,4-oxadiazol-3-yl)piperidin-4-yl)pyrrolidin-2-one;
(S)-3-(2-fluoro-4-(methylsulfonyl)phenylamino)-1′-(5-isopropyl-1,2,4-oxadiazol-3-yl)-1,4′-bipiperidin-2-one;
(S)-3-(2-fluoro-4-(methylsulfonyl)phenylamino)-1-(1-(5-isopropyl-1,2,4-oxadiazol-3-yl)piperidin-4-yl)pyrrolidin-2-one;
(S)-3-(2-fluoro-4-(methylsulfonyl)phenylamino)-1-(1-(5-(trifluoromethyl)-1,2,4-oxadiazol-3-yl)piperidin-4-yl)pyrrolidin-2-one;
(S)-3-(2,5-difluoro-4-(methylsulfonyl)phenoxy)-1-(1-(5-phenyl-1,2,4-oxadiazol-3-yl)piperidin-4-yl)pyrrolidin-2-one;
(S)-3-(2-fluoro-4-(methylsulfonyl)phenoxy)-1-(1-(5-isopropyl-1,2,4-oxadiazol-3-yl)piperidin-4-yl)pyrrolidin-2-one;
(S)-3-(2,5-difluoro-4-(methylsulfonyl)phenoxy)-1-(1-(5-isopropyl-1,2,4-oxadiazol-3-yl)piperidin-4-yl)pyrrolidin-2-one;
(S)-3-(2,6-difluoro-4-(methylsulfonyl)phenoxy)-1-(1-(5-isopropyl-1,2,4-oxadiazol-3-yl)piperidin-4-yl)pyrrolidin-2-one;
(S)-1-(1-(5-(difluoromethyl)-1,2,4-oxadiazol-3-yl)piperidin-4-yl)-3-(2-fluoro-4-(methylsulfonyl)phenylamino)pyrrolidin-2-one;
(S)-3-(2,6-difluoro-4-(methylsulfonyl)phenylamino)-1-(1-(5-(trifluoromethyl)-1,2,4-oxadiazol-3-yl)piperidin-4-yl)pyrrolidin-2-one;
(S)-3-(2,5-difluoro-4-(methylsulfonyl)phenylamino)-1-(1-(5-(trifluoromethyl)-1,2,4-oxadiazol-3-yl)piperidin-4-yl)pyrrolidin-2-one;
(S)-3-(2-fluoro-4-(methylsulfonyl)phenoxy)-1-(1-(5-(trifluoromethyl)-1,2,4-oxadiazol-3-yl)piperidin-4-yl)pyrrolidin-2-one;
(S)-3-(2,5-difluoro-4-(methylsulfonyl)phenylamino)-1′-(5-(trifluoromethyl)-1,2,4-oxadiazol-3-yl)-1,4′-bipiperidin-2-one;
(S)-3-(2,6-difluoro-4-(methylsulfonyl)phenylamino)-1′-(5-(trifluoromethyl)-1,2,4-oxadiazol-3-yl)-1,4′-bipiperidin-2-one;
(S)-3-(2,5-difluoro-4-(methylsulfonyl)phenoxy)-1-(1-(5-(trifluoromethyl)-1,2,4-oxadiazol-3-yl)piperidin-4-yl)pyrrolidin-2-one;
(S)-3-(2,6-difluoro-4-(methylsulfonyl)phenoxy)-1-(1-(5-(trifluoromethyl)-1,2,4-oxadiazol-3-yl)piperidin-4-yl)pyrrolidin-2-one;
(S)-3-(2-fluoro-4-(methylsulfonyl)phenylamino)-1′-(5-(trifluoromethyl)-1,2,4-oxadiazol-3-yl)-1,4′-bipiperidin-2-one;
(S)-3-(2,5-difluoro-4-(methylsulfonyl)phenylamino)-1-(1-(5-(difluoromethyl)-1,2,4-oxadiazol-3-yl)piperidin-4-yl)pyrrolidin-2-one;
(S)-3-(2,6-difluoro-4-(methylsulfonyl)phenylamino)-1-(1-(5-(difluoromethyl)-1,2,4-oxadiazol-3-yl)piperidin-4-yl)pyrrolidin-2-one;
(S)-3-(2,6-difluoro-4-(methylsulfonyl)phenylamino)-1′-(5-(difluoromethyl)-1,2,4-oxadiazol-3-yl)-1,4′-bipiperidin-2-one;
(S)-3-(2,5-difluoro-4-(methylsulfonyl)phenylamino)-1′-(5-(difluoromethyl)-1,2,4-oxadiazol-3-yl)-1,4′-bipiperidin-2-one;
(S)-3-(4-(ethylsulfonyl)-2-fluorophenylamino)-1-(1-(5-(trifluoromethyl)-1,2,4-oxadiazol-3-yl)piperidin-4-yl)pyrrolidin-2-one;
(S)-3-(2,5-difluoro-4-(methylsulfonyl)phenylamino)-1′-(5-(difluoromethyl)-1,2,4-oxadiazol-3-yl)-1,4′-bipiperidin-2-one;
(S)-3-(2-fluoro-4-(methylsulfonyl)phenylamino)-1-(1-(5-(2,2,2-trifluoroethyl)-1,2,4-oxadiazol-3-yl)piperidin-4-yl)pyrrolidin-2-one;
(S)-3-(2-fluoro-4-(methylsulfonyl)phenylamino)-1-(1-(5-propyl-1,2,4-oxadiazol-3-yl)piperidin-4-yl)pyrrolidin-2-one;
(S)-3-(2-fluoro-4-(methylsulfonyl)phenylamino)-1-(1-(5-(pyridin-2-yl)-1,2,4-oxadiazol-3-yl)piperidin-4-yl)pyrrolidin-2-one;
(S)-3-(2-fluoro-4-(methylsulfonyl)phenylamino)-1-(1-(5-(1,1,1-trifluoro-2-methylpropan-2-yl)-1,2,4-oxadiazol-3-yl)piperidin-4-yl)pyrrolidin-2-one;
(S)-1-(1-(5-cyclobutyl-1,2,4-oxadiazol-3-yl)piperidin-4-yl)-3-(2-fluoro-4-(methylsulfonyl)phenylamino)pyrrolidin-2-one;
(S)-1-(1-(5-sec-butyl-1,2,4-oxadiazol-3-yl)piperidin-4-yl)-3-(2-fluoro-4-(methylsulfonyl)phenylamino)pyrrolidin-2-one;
(S)-3-(2-fluoro-4-(methylsulfonyl)phenylamino)-1-(1-(5-(2-fluoropropan-2-yl)-1,2,4-oxadiazol-3-yl)piperidin-4-yl)pyrrolidin-2-one;
(S)-1-(1-(5-cyclopentyl-1,2,4-oxadiazol-3-yl)piperidin-4-yl)-3-(2-fluoro-4-(methylsulfonyl)phenylamino)pyrrolidin-2-one;
(S)-3-(2-fluoro-4-(methylsulfonyl)phenylamino)-1-(1-(5-phenyl-1,2,4-oxadiazol-3-yl)piperidin-4-yl)pyrrolidin-2-one;
(S)-1-(1-(5-cyclopropyl-1,2,4-oxadiazol-3-yl)piperidin-4-yl)-3-(2-fluoro-4-(methylsulfonyl)phenylamino)pyrrolidin-2-one;
(S)-3-(2-fluoro-4-(methylsulfonyl)phenylamino)-1-(1-(5-(1-(trifluoromethyl)cyclopropyl)-1,2,4-oxadiazol-3-yl)piperidin-4-yl)pyrrolidin-2-one;
(S)-1-(1-(5-(1,1-difluoropropyl)-1,2,4-oxadiazol-3-yl)piperidin-4-yl)-3-(2-fluoro-4-(methylsulfonyl)phenylamino)pyrrolidin-2-one;
(S)-3-(2,5-difluoro-4-(methylsulfonyl)phenylamino)-1′-(5-(1,1-difluoroethyl)-1,2,4-oxadiazol-3-yl)-1,4′-bipiperidin-2-one;
(S)-1-(1-(5-(1,1-difluoroethyl)-1,2,4-oxadiazol-3-yl)piperidin-4-yl)-3-(2-fluoro-4-(methylsulfonyl)phenylamino)pyrrolidin-2-one;
(S)-3-(2,6-difluoro-4-(methylsulfonyl)phenylamino)-1′-(5-(1,1-difluoroethyl)-1,2,4-oxadiazol-3-yl)-1,4′-bipiperidin-2-one;
((S)-3-(2-fluoro-4-(methylsulfonyl)phenylamino)-1-(1-(5-(pyrrolidin-1-yl)-1,2,4-oxadiazol-3-yl)piperidin-4-yl)pyrrolidin-2-one;
(S)-3-(2,5-difluoro-4-(methylsulfonyl)phenoxy)-1-(1-(5-(1,1-difluoroethyl)-1,2,4-oxadiazol-3-yl)piperidin-4-yl)pyrrolidin-2-one;
(S)-3-(2,5-difluoro-4-(methylsulfonyl)phenylamino)-1-(1-(5-(trifluoromethyl)-1,2,4-oxadiazol-3-yl)piperidin-4-yl)azepan-2-one;
(S)-3-(2,5-difluoro-4-(methylsulfonyl)phenoxy)-1′-(5-(trifluoromethyl)-1,2,4-oxadiazol-3-yl)-[1,4′-bipiperidin]-2-one;
(S)-3-(2,5-difluoro-4-(methylsulfonyl)phenoxy)-1′-(5-(1,1-difluoroethyl)-1,2,4-oxadiazol-3-yl)-[1,4′-bipiperidin]-2-one;
(S)-3-((6-(methylsulfonyl)pyridin-3-yl)oxy)-1-(1-(5-(trifluoromethyl)-1,2,4-oxadiazol-3-yl)piperidin-4-yl)pyrrolidin-2-one;
(S)-3-((6-(methylsulfonyl)pyridin-3-yl)amino)-1-(1-(5-(trifluoromethyl)-1,2,4-oxadiazol-3-yl)piperidin-4-yl)pyrrolidin-2-one;
(S)-1-(1-(5-(difluoromethyl)-1,2,4-oxadiazol-3-yl)piperidin-4-yl)-3-((6-(methylsulfonyl)pyridin-3-yl)amino)pyrrolidin-2-one;
(S)-3-((2,5-difluoro-4-(methylsulfonyl)phenyl)amino)-1-(1-(3-(trifluoromethyl)-1,2,4-oxadiazol-5-yl)piperidin-4-yl)pyrrolidin-2-one;
(S)-3-((2,6-difluoro-4-(methylsulfonyl)phenyl)amino)-1-(1-(3-(trifluoromethyl)-1,2,4-oxadiazol-5-yl)piperidin-4-yl)pyrrolidin-2-one;
(S)-3-((2,5-difluoro-4-(methylsulfonyl)phenyl)amino)-1′-(3-(1,1-difluoroethyl)-1,2,4-oxadiazol-5-yl)-[1,4′-bipiperidin]-2-one;
(S)-3-((2,6-difluoro-4-(methylsulfonyl)phenyl)amino)-1′-(3-(1,1-difluoroethyl)-1,2,4-oxadiazol-5-yl)-[1,4′-bipiperidin]-2-one;
(S)-3-(2,5-difluoro-4-(methylsulfonyl)phenoxy)-1-(1-(3-(1,1-difluoroethyl)-1,2,4-oxadiazol-5-yl)piperidin-4-yl)pyrrolidin-2-one;
(S)-3-((2,5-difluoro-4-(methylsulfonyl)phenyl)amino)-1-(1-(5-(1,1-difluoroethyl)-1,2,4-oxadiazol-3-yl)piperidin-4-yl)pyrrolidin-2-one;
(S)-3-((2,6-difluoro-4-(methylsulfonyl)phenyl)amino)-1-(1-(5-(1,1-difluoroethyl)-1,2,4-oxadiazol-3-yl)piperidin-4-yl)pyrrolidin-2-one;
(R)-3-(2,5-difluoro-4-(methylsulfonyl)phenoxy)-1-(1-(5-(trifluoromethyl)-1,2,4-oxadiazol-3-yl)piperidin-4-yl)pyrrolidin-2-one;
(R)-3-(2,5-difluoro-4-(methylsulfonyl)phenoxy)-1′-(5-(trifluoromethyl)-1,2,4-oxadiazol-3-yl)-[1,4′-bipiperidin]-2-one;
or a pharmaceutically acceptable salt thereof.

41. A pharmaceutical composition, which comprises a compound of Formula I as defined in claim 1 or a pharmaceutically acceptable salt thereof, and a pharmaceutically acceptable diluent, carrier or excipient.

42. A method of treating a disease or condition selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis in a mammal, which comprises administering to said mammal a therapeutically effective amount of a compound of Formula I as defined in claim 1 or a pharmaceutically acceptable salt thereof.

43. The method of claim 42, wherein the disease is type 2 diabetes.

44. (canceled)

45. (canceled)

46. A process for the preparation of a compound of claim 1, which comprises:
(a) for a compound of Formula I wherein R⁷ is 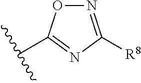
and R⁸ is as defined for Formula I, cyclizing a corresponding compound having the formula II 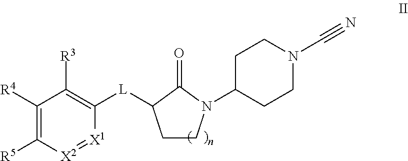
wherein X¹, X², L, R³, R⁴, R⁵ and n are as defined for Formula I, with a reagent having the formula 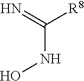
wherein R⁸ is defined for Formula I, in the presence of a Lewis acid; or
(b) for a compound of Formula I wherein R⁷ is 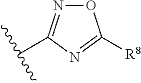
and R⁸ is as defined for Formula I, cyclizing a corresponding compound having the formula III 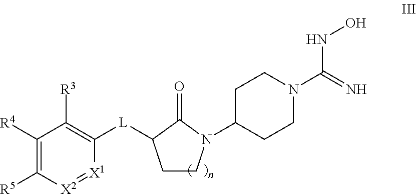
wherein X¹, X², L, R³, R⁴, R⁵ and n are as defined for Formula I, with a reagent having the formula R⁸C(═O)OH or a reactive derivative thereof wherein R⁸ is as defined for Formula I, optionally in the presence of a base; or
(c) for a compound of Formula I wherein R⁷ is 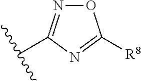
and R⁸ is hetCyc¹, coupling a corresponding compound having the formula IV 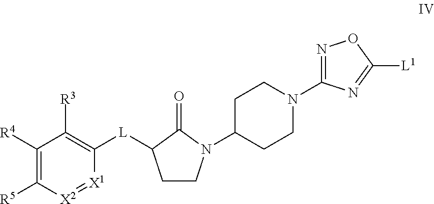
wherein X¹, X², L, R³, R⁴, R⁵ and n are as defined for Formula I and L¹ is a leaving group or atom, with a reagent having the structure: 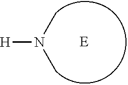
where Ring E is as defined for hetCyc¹, in the presence of a base; and
optionally removing any protecting groups and optionally preparing a salt thereof.

47. A compound according to claim 40, which is (S)-3-(2,5-difluoro-4-(methylsulfonyl)phenylamino)-1-(1-(5-(trifluoromethyl)-1,2,4-oxadiazol-3-yl)piperidin-4-yl)pyrrolidin-2-one, or a pharmaceutically acceptable salt thereof.

48. A compound according to claim 40, which is (S)-3-(2,5-difluoro-4-(methylsulfonyl)phenoxy)-1-(1-(5-(trifluoromethyl)-1,2,4-oxadiazol-3-yl)piperidin-4-yl)pyrrolidin-2-one, or a pharmaceutically acceptable salt thereof.

49. A compound according to claim 40, which is (S)-3-(2,5-difluoro-4-(methylsulfonyl)phenoxy)-1-(1-(5-(1,1-difluoroethyl)-1,2,4-oxadiazol-3-yl)piperidin-4-yl)pyrrolidin-2-one, or a pharmaceutically acceptable salt thereof.

50. A compound according to claim 40, which is (S)-3-(2,5-difluoro-4-(methylsulfonyl)phenoxy)-1′-(5-(trifluoromethyl)-1,2,4-oxadiazol-3-yl)-[1,4′-bipiperidin]-2-one, or a pharmaceutically acceptable salt thereof.

51. A compound according to claim 5, wherein R⁵ is (1-3C)alkylsulfonyl.

52. A compound according to claim 51, wherein R⁷ is

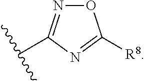

53. A compound according to claim 52, wherein R⁸ is selected from 2-fluoropropyl, difluoromethyl, 1,1-difluoroethyl, 1,1-difluoropropyl, trifluoromethyl and 1,1-dimethyl-2,2-difluoroethyl.

54. A compound according to claim 53, wherein L is O.

55. A compound according to claim 54, wherein n is 1.

56. A compound according to claim 55 having the absolute configuration of Formula I-a:

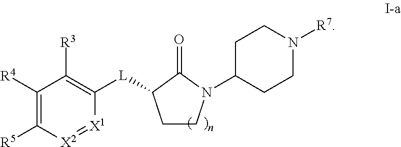

57. A compound having the formula III 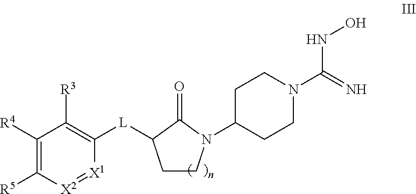
wherein:
L is O or NR^{x};
R^{x} is H or (1-3C)alkyl;
X¹ is N or CR¹ and X² is N or CR², wherein only one of X¹ and X² may be N;
R¹, R², R³ and R⁴ are independently selected from H, halogen, CF₃, (1-6C)alkyl and (1-6C)alkoxy;
R⁵ is (1-3C alkyl)sulfonyl, (3-6C cycloalkyl)sulfonyl, (cyclopropylmethyl)sulfonyl, phenylsulfonyl, CN, Br, CF₃, or tetrazolyl optionally substituted with (1-3C)alkyl; and
n is 1, 2 or 3.
